(19) United States
(12) Patent Application Publication
(10) Pub. No.: US 2017/0152245 A1
(43) Pub. Date: Jun. 1, 2017

(54) BENZIMIDAZOLE COMPOUNDS AND THEIR APPLICATION IN CARDIOVASCULAR DISEASES

(71) Applicant: National Taiwan University, Taipei City (TW)

(72) Inventors: Tur-Fu Huang, Taipei city (TW); Shiu-Wen Huang, Taipei city (TW); Jin-Cherng Lien, Taichung City (TW); Sheng-Chu Kuo, Taichung City (TW)

(73) Assignee: National Taiwan University, TAIPEI CITY (TW)

(21) Appl. No.: 15/361,093

(22) Filed: Nov. 25, 2016

Related U.S. Application Data
(60) Provisional application No. 62/260,263, filed on Nov. 26, 2015.

(51) Int. Cl. C07D 405/04; C07D 409/04

(52) U.S. Cl. CPC C07D 405/04; C07D 409/04

(57) ABSTRACT

The present invention provides substituted benzimidazoles, pharmaceutical composition comprising the substituted benzimidazoles, and their use for preventing or treating a subject suffering from diseases or conditions associated with platelet activation aggregation and/or platelet activation.

### FIELD OF THE INVENTION

The present invention provides substituted benzimidazoles, pharmaceutical composition comprising the substituted benzimidazoles, and their use for preventing or treating a subject suffering from diseases or conditions associated with platelet activation aggregation and/or platelet activation.

### DESCRIPTION OF PRIOR ART

Cardiovascular diseases may result from a number of pathophysiologic processes involving blood vessels, including intrinsic disease such as atherosclerosis, inflammation, arterial dissection, or reduction of cerebral perfusion, embolus or hemorrhage from a vessel rupture, and myocardial infarction and ischemic stroke are the leading causes of morbidity and mortality in the developed countries. Atherothrombosis is the most common cause of acute ischemic stroke and therefore its prevention will reduce the incidence of ischemic stroke. Among the numerous processes involved in atherothrombosis, activation and aggregation of platelets and thrombus formation play an important role, involving platelet adhesion, activation and subsequent aggregation. Antiplatelet agents, such as aspirin, have been shown to be effective in the primary and secondary prevention of cardiovascular events, including stroke.

Furthermore, appropriate platelet adhesion, activation and aggregation are important in maintaining a balance between normal hemostasis and pathological arterial thrombosis such as stroke and myocardial infarction. Exposure of matrix protein collagen after vessel injury provides a substrate for platelet adhesion and triggers platelet activation, which recruits additional platelets to area of injured vessel wall, thereby initiating thrombus formation. Platelet adhesion and aggregation are critical events in intravascular thrombosis. The formation of a blood clot is normally the result of tissue injury which initiates platelet adhesion/aggregation and coagulation cascade and has the effect of slowing or preventing blood flow in wound healing. However, in certain disease states the formation of blood clots within the circulatory system reaches an undesired extent and is itself the source of morbidity potentially leading to pathological consequences.

On the other hand, abnormal intimal growth, vascular smooth muscle cell (VSMC) proliferation and migration from the media to the intima are key events in the pathogenesis of restenosis after balloon angioplasty and stenting procedures. The pathophysiology of restenosis involves accumulation of new tissue within the arterial wall, and the process can be divided into several stages, including the SMCs migration from the media, crossing through the injured endothelium, and proliferation into generating the neointima, which clogs the blood vessel.

Furthermore, upon activation under conditions of turbulent blood flow in diseased vessels or by the release of mediators from other circulating cells and damaged endothelial cells lining the vessel, platelets accumulate at a site of vessel injury and recruit further platelets into the developing thrombus. The thrombus can grow to sufficient size to block off arterial blood vessels.

There is still a need of developing new selective, efficacious antiplatelet agents and/or novel compounds with fewer side effects for the treatment and prevention of cardiovascular disease is under active investigation.

The details of one or more embodiments of the invention are set forth in the description below. Other features, objects, and advantages of the invention will be apparent from the description and the claims.

### BRIEF SUMMARY OF THE INVENTION

The present invention provides a substituted benzimidazole having a chemical structure (I):

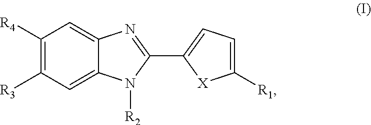

wherein X is selected from oxygen, carbon, or sulfur; R₁ is C₁₋₆ alkyl; R₂ is selected from hydrogen, C₁₋₆ alkyl, C₁₋₆ alkyl pyridine, unsubstituted benzyl, or benzyl substituted with one or more C₁₋₆ alkyl, C₁₋₆ alkoxy, halo, or nitro; and R₃ and R₄ are independently selected from hydrogen, C₁₋₆ alkoxy, halogen, phenylcarbonyl, or C₁₋₆ alkylcarbonyl.

The present invention also provides a pharmaceutical composition comprising: an effective amount of a compound having a chemical structure (I):

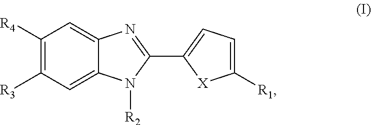

wherein X is selected from oxygen, carbon, or sulfur; R₁ is C₁₋₆ alkyl; R₂ is selected from hydrogen, C₁₋₆ alkyl, C₁₋₆ alkyl pyridine, unsubstituted benzyl, or benzyl substituted with one or more C₁₋₆ alkyl, C₁₋₆ alkoxy, halo, or nitro; and R₃ and R₄ are independently selected from hydrogen, C₁₋₆ alkoxy, halogen, phenylcarbonyl, or C₁₋₆ alkylcarbonyl.

The present invention further provides a method of preventing or treating a subject suffering from diseases associated with thromboxane A₂ by inhibiting thromboxane A₂ activity, comprising: administering an effective amount of a compound having a chemical structure (I) to the subject:

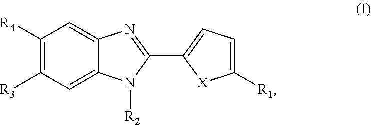

wherein X is selected from oxygen, carbon, or sulfur; R₁ is C₁₋₆ alkyl; R₂ is selected from hydrogen, C₁₋₆ alkyl, C₁₋₆ alkyl pyridine, unsubstituted benzyl, or benzyl substituted with one or more C₁₋₆ alkyl, C₁₋₆ alkoxy, halo, or nitro; and R₃ and R₄ are independently selected from hydrogen, C₁₋₆ alkoxy, halogen, phenylcarbonyl, or C₁₋₆ alkylcarbonyl.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Effects of nstpbp5185 on collagen- or arachidonic acid-induced platelet aggregation. (A) Platelets were pre-incubated with indicated concentrations of nstpbp5185 or aspirin at 37° C. for 3 min, then collagen (10 μg ml⁻¹, ∇) was added to trigger platelet aggregation in platelet suspension (PS). Typical tracing curves shown are representative of six independent experiments. The IC₅₀ values of nstpbp5185 and aspirin are shown in (B). (C) Platelets were pre-incubated with indicated concentrations of nstpbp5185 or aspirin at 37° C. for 3 min, then arachidonic acid (AA, 200 μM, ∇) was added to trigger platelet aggregation in PS. Typical tracing curves shown are representative of six independent experiments. The IC₅₀ values of nstpbp5185 and aspirin are shown in (D).

FIG. 2 shows the effect of Ctkf6f2 on the aggregation of washed human platelets. Effect of Ctkf6f2 on the platelet aggregation caused by (A) U46619 (1 μM) (B) collagen (10 μg/ml), or (C) arachidonic acid (a.a., 200 μM) in washed human platelets. Platelet suspension were preincubated with DMSO (control) or Ctkf6f2 at 37° C. in an aggregometer with stirring at 900 rpm for 3 min, and U46619, collagen or arachidonic acid (a.a.) was added (▴) to trigger aggregation, respectively. Platelet aggregation was measured turbidimetrically (ΔT) using a platelet aggregometer. The result is representative of three experiments.

FIG. 3 shows the concentration-dependent inhibition curves of Ctkf6f2 in U46619, collagen and arachidonic acid-induced platelet aggregation of (A) platelet suspension and (B) platelet-rich plasma. (A) Washed human platelet suspension or (B) platelet-rich plasma were preincubated with DMSO or Ctkf6f2, and U46619, collagen or arachidonic acid (a.a.) was added to trigger platelet aggregation. Data are presented as a percentage of the control (mean±S.E.M., n=3).

FIG. 4 Effects of nstpbp5185 on intracellular Ca²⁺ mobilization, P-selectin expression and thromboxane B2 production of platelets in human platelet suspension. (A) Collagen (10 μg ml⁻¹) or (B) thrombin (0.1 U ml⁻¹) was added to Fura-2-loaded platelets in the absence or presence of various concentrations of nstpbp5185, which were preincubated with platelets for 3 min. (C) The solid line represents the fluorescence profiles of FITC-labeled anti-CD62P (10 μg ml⁻¹) in collagen (10 μg ml⁻¹)-stimulated platelets or in U46619 (1 μM)-stimulated platelets (D). Data are presented as the means±SEM. The profiles are representative examples of three similar experiments. (E) Washed platelets were preincubated with nstpbp5185 (0.7, 2 and 6 μM) or aspirin (500 μM) for 3 min at 37° C., and then collagen (10 μg ml⁻¹) or (F) arachidonic acid (200 μM) was added to trigger thromboxane B2 formation. Data are presented as means±SEM (n=3). ***p<0.001 as compared with the collagen or arachidonic acid control group.

FIG. 5 shows the effects of Ctkf6f2 on (A) collagen or (B) U46619-induced intracellular Ca²⁺ mobilization in human platelet. Fura-2-loaded platelets were suspended in Tyrode buffer containing 1 mM CaCl₂, and the changes in [Ca²⁺]ᵢ were continuously monitored. Platelets were preincubated with Ctkf6f2 at 37° C. for 3 min, then (A) collagen (10 μg/ml) or (B) U46619 (1 μM) was added. Data shown are representative of three independent experiments.

FIG. 6 shows the effect of Ctkf6f2 on (A) collagen, (B) U46619-induced P-selectin expression. Washed human platelets were preincubated with Ctkf6f2 and treated with (A) collagen (10 μg/ml) or (B) U46619 (1 μM) in the presence of control IgG or FITC-conjugated anti-CD62P mAbs. P-selectin expression was performed by flow cytometric analysis and expressed as percentage of expression compared with positive control. (C) The effect of Ctkf6f2 on collagen-induced TXB₂ formation. Platelets suspensions were preincubated with Ctkf6f2 (0.3, 1, 3, 10 μM) or nstpbp5185 (1 μM) for 3 min at 37° C., and then collagen (10 μg/ml) was added to trigger TXB₂ formation. Data are presented as the mean±S.E.M. (n=3). ***p<0.001 and **p<0.01 as compared with the control, one-way ANOVA (Newman-Keuls multiple comparison test).

FIG. 7 Effects of nstpbp5185 on human platelet aggregation induced by U46619. (A) Platelets were pre-incubated with nstpbp5185 or indomethacin (Indo) at 37° C. for 3 min, then U46619 (1 μM, ∇) was added to trigger platelet aggregation. Typical tracing curves shown are representative of four independent experiments. (B) Indomethacin (50 μM)-treated human platelets (for 3 min) were used to exclude any possible contribution of endogenous arachidonic acid metabolites to platelet aggregation. Washed human platelets were incubated with nstpbp5185 or DMSO (0.1%) at 37° C. for 3 min, and then U46619 was added to trigger aggregation. The peak level of aggregation was measured for 4 min after the addition of U46619. The maximum value of the control produced by U46619 (10 μM) was taken as 100%. Each data point is expressed as mean±S.E.M. (n=4). (C) Effect of nstpbp5185 on [³H] SQ-29548 binding to TP receptor of recombinant HEK-293 cells.

FIG. 8 shows the competitive inhibitory curve of nstpbp5185 and Ctkf6f2 on U46619-induced platelet aggregation. Platelet suspension were preincubated with nstpbp5185 (1 μM) or Ctkf6f2 (1 μM), U46619 in different concentration as shown was added to trigger the aggregation. Platelet aggregation was measured using a platelet aggregometer. The result is representative of three experiments. The percentage of aggregation was calculated relative to the maximum value of the control (in the absence of nstpbp5185 or Ctkf6f2) produced by U46619. Each data point is expressed as mean±SEM (n=3).

FIG. 9 shows the effects of Ctkf6f2 on irradiated fluorescent dye-induced platelet-rich thrombus formation in mesenteric venules of mice. Mice were intravenously administered with vehicle control, nstpbp5185 (5 or 10 μg/g) or Ctkf6f2 (5 or 10 μg/g). The mesenteric venules were selected for light irradiation to produce microthrombus formation as described in Materials and methods. The effect of time to occlusion (TTO) was measured 5 min after i.v. administration. The average TTO is indicated as (−). Each different symbol represents the time to occlusion of the mouse. ***p<0.001 as compared with the control, one-way ANOVA (Newman-Keuls multiple comparison test).

FIG. 10 shows the effects of Ctkf6f2 on mouse PRP ex vivo aggregation caused by U46619. Mice were i.v. injected with vehicle, nstpbp5185 (10 μg/g) or Ctkf6f2 (10 μg/g). The blood samples were collected within 10 mins by intracardiac puncture for PRP, and U46619 (0.125 μM) was added. Platelet aggregation was measured turbidimetrically using platelet aggregometer. Shown are representative of three independent experiments.

FIG. 11 shows the effects of oral administration of nstpbp5185 on platelet aggregation of PRP caused by collagen. Mice were orally treated with vehicle, nstpbp5185 (40 and 70 mg/kg/day) for 10 days. The blood samples were collected by intracardiac puncture. PRP was prepared and adjusted to 3×10⁸ platelets/ml, collagen (10 μg/ml) was added as aggregation inducer. Platelet aggregation was measured turbidimetrically using platelet aggregometer. Aggregation traces shown are representative of three independent experiments.

FIG. 12 Inhibitory effect of nstpbp5185 on fluorescent dye-induced platelet-rich thrombus formation in mesenteric venules and tail bleeding times of mice and gastric ulcerogenic effects in rats. Effect of nstpbp5185 on the time to occlusion (TTO) measured 5 min after its i.v. administration upon light irradiation of mesenteric venules of mice pretreated with fluorescein sodium. Data are presented as the mean±SEM (n=10-15). (B) Effect of nstpbp5185 on tail bleeding time of mice measured 5 min after i.v. administration. Data are presented as the mean±SEM (n=15). (C) The formalin-fixed stomach of aspirin or nstpbp5185-treated rat was shown in upper panel and the number of lesion >2 mm caused by acute oral administration of aspirin (150 μg g⁻¹) or nstpbp5185 (40 μg g⁻¹) was shown in lower panel (n=3). *P<0.05, **P<0.01 and ***P<0.001 as compared with the vehicle control (DMSO).

FIG. 13 Effect of nstpbp5185 on the mortality rate and lung thromboembolism caused by collagen/epinephrine. (A) Male ICR mice received collagen (0.6 μg g⁻¹) plus epinephrine (0.2 μg g⁻¹) by tail vein injection. DMSO (vehicle) or nstpbp5185 (5 or 10 μg g⁻¹) was intravenously given 5 min prior to collagen/epinephrine. (B) Lung thromboembolism was found in vehicle-treated mice (left panel) whereas thromboembolism-free was noted in nstpbp5185-treated mice (middle, and right panel). (C) Thromboembolism area (mm²) in 200× filed are presented as the mean±SEM (n≧4). ***P<0.001 as compared with the vehicle control (DMSO). (D) Mortality rate was evaluated 1 hour after challenge (n≧4).

FIG. 14 The antithrombotic activity of nstpbp5185 in FeCl₃-induced thrombi formation in mice carotid artery. (A) Mice were intravenously administered with Heparin (10 or 100 U kg⁻¹), Aspirin (40 or 80 μg g⁻¹), or different doses of nstpbp5185 (B). After 5 min, FeCl₃ injury was induced by a filter paper saturated with ferric chloride solution (10%). After removal of the paper, carotid blood flow (ml min⁻¹) was monitored continuously until thromboembolism formation or for 60 min. (C) Mice were orally administered with different doses of Aspirin and nstpbp5185 (D). Both drugs were suspended in 1% CMC. Blood flow monitoring were recorded used analyzed Labchart software (AD instruments). Data are presented as the mean±SEM (n≧3). **P<0.01 and ***P<0.001 as compared with the vehicle control (DMSO).

FIG. 15 shows the comparative studies of nstpbp5185 and Ctkf6f2 on the mortality rate and pulmonary thromboembolism. (A) Mice were i.v. injected with vehicle (DMSO as control), nstpbp5185 (10 μg/g) or Ctkf6f2 (10 μg/g), then injected with collagen/epinephrine to induce pulmonary embolism model. After the onset of respiratory arrest, lungs were excised. The lungs are fixed in 24% formalin, and embedded into paraffin. Histologic analysis was performed on H&E-stained sections of lung from each mouse. The arrow indicates the blood vessel thrombi. (B) Survival rate of mice injected with DMSO as control, nstpbp5185 (10 μg/g) or Ctkf6f2 (10 μg/g) after induction of pulmonary thromboembolism in 30 min, 1 hr and 24 hr (n=4). (C) Representative images of lung isolated after the onset of respiratory arrest, but while the heart was still beating, 0.5 ml of Evans blue solution was injected into the heart. Lungs were excised, and photographed.

FIG. 16 shows the inhibitory effect of nstpbp5185 on neointima formation after carotid artery injury. The neointima formation was attenuated in the nstpbp5185-treated group compared with the control group. As shown in FIG. 17B, the sham (noninjuried) group was free of intimal thickening. After balloon catheter injury of the artery in the control group, there was evident neointima thickening (FIG. 17A) 14 days after the operation. On the other hand, the vessels after nstpbp5185 treatment at various doses (FIGS. 17C and 17E) exhibited reduction of neointimal formation as compared with the control. The results showed that nstpbp5185 significantly inhibited the N/M ratio (neointima/media) as it was administered intraperitoneally at 1 or 2 mg/kg/day for 2 weeks (FIG. 17G).

FIG. 17 Aortic root lesion area in vehicle-, aspirin-, and nstpbp5185-treated Apo E-deficient mice. (A) Eight-week-old Apo E-deficient mice were orally given with vehicle, aspirin or nstpbp5185 at indicated dosage. After 12 weeks, each individual aortic root cross section was sliced, stained with Hematoxylin and Eosin (HE) and analyzed under microscopy. Representative photomicrographs of aortic root obtained from mice were shown. Original magnification was ×40. Mice were treated as described in (A), intima/media ratio for each individual aortic root cross section shown at the bottom (B) and digitized lesion area in 2 mm per cross-section in vehicle-, aspirin-, and nstpbp5185-treated Apo E-deficient mice were then evaluated (C). Data are presented as the means±S.E.M. *P<0.05, **P<0.01, ***P<0.001, compared to the control group. (D) Aortic lesion areas of Apo E-deficient mice by en face preparation. Eight-week-old Apo E-deficient mice were orally given with vehicle, aspirin and nstpbp5185 at indicated dosage. After 12 weeks, aortic lesion area was then analyzed. Representative photomicrographs of aorta vessel from each group of mice were shown. Original magnification was ×10.

FIG. 18 Nstpbp5185 treatment alleviates AHR and attenuates immunoglobulin release and lung infiltration in OVA-induced mice. (A-C) OVA-specific serum IgE, IgG2a and IgG1 levels. After treatment with nstpbp5185, there was significant difference in serum Ig between the control and nstpbp5185 treatment groups. Values are expressed as mean±SEM. (One-way ANOVA was followed by the Newman-Keuls test *P<0.05; compared to control) Ig, immunoglobulin; OVA, ovalbumin. (D) Mice were immunized, treated, and challenged as described in FIG. 1. Airway responses to methacholine were measured with whole body plethysmograph (Buxco Electronics, Inc., Sharon, Conn., USA) apparatus 24 h after i.n. OVA challenge. Data are presented as the ratio of the enhanced pause (Penh) at a given dose of methacholine compared to that obtained with PBS. Data shown are representative of 2 experiments. *P□<□0.05 versus OVA group. (E) Histological analysis of pulmonary sections from immunized mice with or without nstpbp5185 treatment. Basal: Unsensitized mice show healthy pulmonary tissue. Control: Mice sensitized and challenged with OVA show cells infiltrating the airways. After nstpbp5185 treatment (10 and 20 mg/kg/day), cellular infiltration was reduced. Sections are stained with H&E. Original magnification, ×100; H&E, hematoxylin-eosin; OVA, ovalbumin.

FIG. 19 Effects of nstpbp5185 on U46619-stimulated protein tyrosine phosphorylation in platelet suspension. Platelets were incubated with nstpbp5185 (2.5 μM) or solvent control for 3 min and then stimulated with U46619 (1 μM) for the times indicated (second, s). Then platelets were lysed in SDS buffer and the lysates separated by SDS-PAGE (7.5%-15% acrylamide gradient) under reducing conditions. After electrophoresis, the proteins were transferred to PVDF membranes, and were incubated with several antiphosphotyrosine mAb before detection by using a peroxidase-linked second Ab and detected by chemiluminescence. Data are representative of three separate experiments.

FIG. 20 Interactions between nstpbp5185 and thromboxane receptor. (A, C) Top view and side view of the interactions, respectively. (Grey: Thromboxane receptor, Red: nstpbp5185, Green: Trp2) (H1: Helix 1, H2: Helix 2, H3: Helix 3, H4: Helix 4, H5: Helix 5, H6: Helix 6, H7: Helix 7). (B, D) 2D ligand interaction diagram. Trp2 forms hydrogen bond and pi-pi stacking interaction with nstpbp5185. Gly9, Pro10, and Val176 play the role of hydrophobic contact.

### DETAILED DESCRIPTION OF THE INVENTION

The present invention provides a substituted benzimidazole having a chemical structure (I):

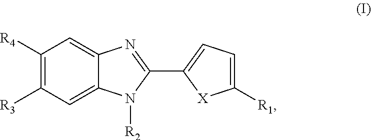

wherein X is selected from oxygen, carbon, or sulfur; R₁ is C₁₋₆ alkyl; R₂ is selected from hydrogen, C₁₋₆ alkyl, C₁₋₆ alkyl pyridine, unsubstituted benzyl, or benzyl substituted with one or more C₁₋₆ alkyl, C₁₋₆ alkoxy, halo, or nitro; and R₃ and R₄ are independently selected from hydrogen, C₁₋₆ alkoxy, halogen, phenylcarbonyl, or C₁₋₆ alkylcarbonyl.

In one embodiment, X is oxygen; and R₁ is methyl. In another embodiment, the benzyl substituted with one or more C₁₋₆ alkyl, C₁₋₆ alkoxy, halo, or nitro is in ortho-position, meta-position, or para-position.

The present invention also provides a pharmaceutical composition comprising: an effective amount of a compound having a chemical structure (I):

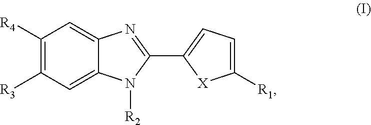

wherein X is selected from oxygen, carbon, or sulfur; R₁ is C₁₋₆ alkyl; R₂ is selected from hydrogen, C₁₋₆ alkyl, C₁₋₆ alkyl pyridine, unsubstituted benzyl, or benzyl substituted with one or more C₁₋₆ alkyl, C₁₋₆ alkoxy, halo, or nitro; and R₃ and R₄ are independently selected from hydrogen, C₁₋₆ alkoxy, halogen, phenylcarbonyl, or C₁₋₆ alkylcarbonyl.

In one embodiment, X is oxygen; and R₁ is methyl. In another embodiment, the benzyl substituted with one or more C₁₋₆ alkyl, C₁₋₆ alkoxy, halo, or nitro is in ortho-position, meta-position, or para-position. In another embodiment, the compound having a chemical structure (I) is:

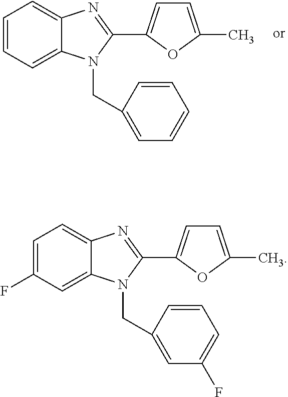 [SMILES: Cc1ccc(-c2nc3ccccc3n2Cc2ccccc2)o1 ; "or" ; Cc1ccc(-c2nc3ccc(F)cc3n2Cc2cccc(F)c2)o1]

The present invention further provides a method of preventing or treating a subject suffering from diseases associated with thromboxane A₂ by inhibiting thromboxane A₂ activity, comprising: administering an effective amount of a compound having a chemical structure (I) to the subject:

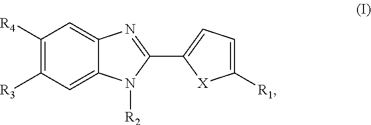

wherein X is selected from oxygen, carbon, or sulfur; R₁ is C₁₋₆ alkyl; R₂ is selected from hydrogen, C₁₋₆ alkyl, C₁₋₆ alkyl pyridine, unsubstituted benzyl, or benzyl substituted with one or more C₁₋₆ alkyl, C₁₋₆ alkoxy, halo, or nitro; and R₃ and R₄ are independently selected from hydrogen, C₁₋₆ alkoxy, halogen, phenylcarbonyl, or C₁₋₆ alkylcarbonyl.

In one embodiment, the diseases associated with thromboxane A₂ comprises inflammatory diseases, coronary artery diseases, percutaneous transluminal coronary angioplasty, and diseases associated with platelet activation aggregation and/or platelet activation.

In one embodiment, X is oxygen; and R₁ is methyl. In another embodiment, the benzyl substituted with one or more C₁₋₆ alkyl, C₁₋₆ alkoxy, halo, or nitro is in ortho-position, meta-position, or para-position. In another embodiment, the compound having a chemical structure (I) is:

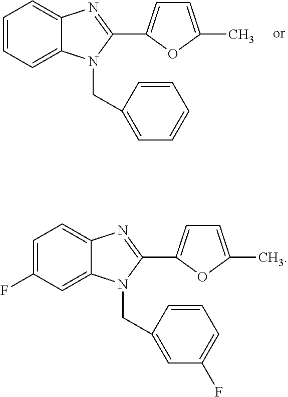 [SMILES: Cc1ccc(-c2nc3ccccc3n2Cc2ccccc2)o1 ; "or" ; Cc1ccc(-c2nc3ccc(F)cc3n2Cc2cccc(F)c2)o1]

In another embodiment, the diseases associated with platelet activation aggregation and/or platelet activation comprise thrombosis, established peripheral arterial disease, thrombophlebitis, arterial embolism, coronary and cerebral arterial thrombosis, unstable angina, myocardial infarction, stroke, cerebral embolism, renal embolism, pulmonary embolism, unstable angina, myocardial infarction, thrombotic stroke, or peripheral vascular disease.

In another embodiment, the inflammatory diseases comprise asthma, and atheroscelrosis.

Shown below are exemplary benzimidazole compounds of the present invention:

| No. | Compound Structure |
|---|---|
| 1 | 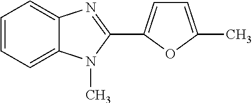 [SMILES: Cc1ccc(-c2nc3ccccc3n2C)o1] |
| 2 | 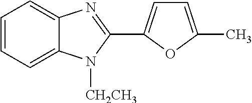 [SMILES: CCn1c(-c2ccc(C)o2)nc2ccccc21] |
| 3 | 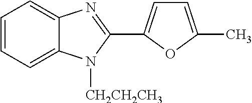 |
| 4 | 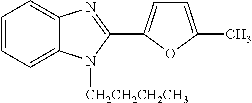 |
| 5 | 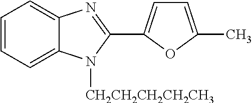 |
| 6 | 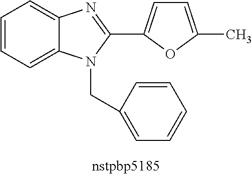 [SMILES: Cc1ccc(-c2nc3ccccc3n2Cc2ccccc2)o1] |
| 7 | 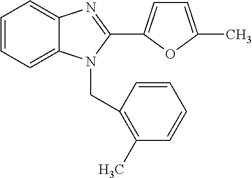 [SMILES: Cc1ccc(-c2nc3ccccc3n2Cc2ccccc2C)o1] |
| 8 | 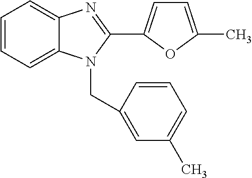 [SMILES: Cc1cccc(Cn2c(-c3ccc(C)o3)nc3ccccc32)c1] |
| 9 | 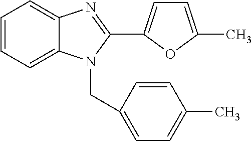 [SMILES: Cc1ccc(Cn2c(-c3ccc(C)o3)nc3ccccc32)cc1] |
| 10 | 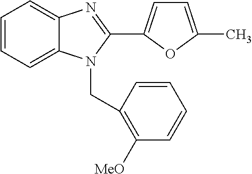 [SMILES: COc1ccccc1Cn1c(-c2ccc(C)o2)nc2ccccc21] |
| 11 | 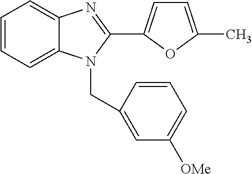 [SMILES: COc1cccc(Cn2c(-c3ccc(C)o3)nc3ccccc32)c1] |
| 12 | 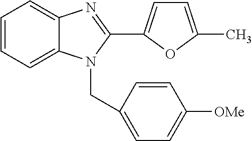 [SMILES: COc1ccc(Cn2c(-c3ccc(C)o3)nc3ccccc32)cc1] |
| | 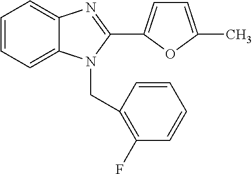 [SMILES: Cc1ccc(-c2nc3ccccc3n2Cc2ccccc2F)o1] |
| 14 | 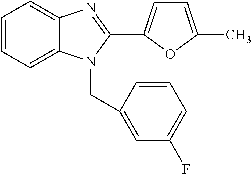 [SMILES: Cc1ccc(-c2nc3ccccc3n2Cc2cccc(F)c2)o1] |
| 15 | 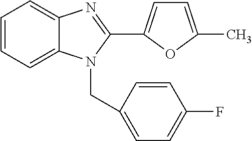 [SMILES: Cc1ccc(-c2nc3ccccc3n2Cc2ccc(F)cc2)o1] |
| 16 | 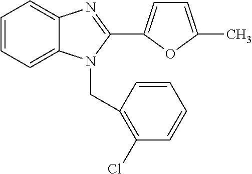 [SMILES: Cc1ccc(-c2nc3ccccc3n2Cc2ccccc2Cl)o1] |
| 17 | 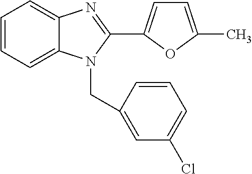 [SMILES: Cc1ccc(-c2nc3ccccc3n2Cc2cccc(Cl)c2)o1] |
| 18 | 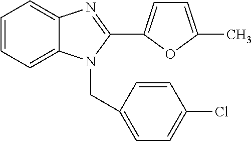 [SMILES: Cc1ccc(-c2nc3ccccc3n2Cc2ccc(Cl)cc2)o1] |
| 19 | 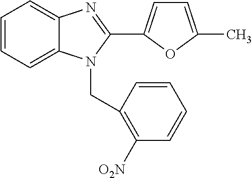 [SMILES: Cc1ccc(-c2nc3ccccc3n2Cc2ccccc2[N+](=O)[O-])o1] |
| 20 | 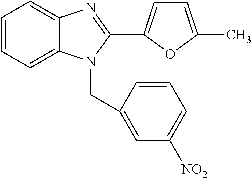 [SMILES: Cc1ccc(-c2nc3ccccc3n2Cc2cccc([N+](=O)[O-])c2)o1] |
| 21 | 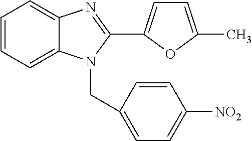 [SMILES: Cc1ccc(-c2nc3ccccc3n2Cc2ccc([N+](=O)[O-])cc2)o1] |
| 22 | 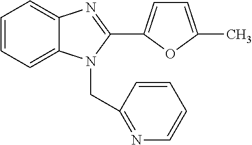 [SMILES: Cc1ccc(-c2nc3ccccc3n2Cc2ccccn2)o1] |
| 23 | 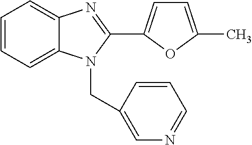 [SMILES: Cc1ccc(-c2nc3ccccc3n2Cc2cccnc2)o1] |
| 24 | 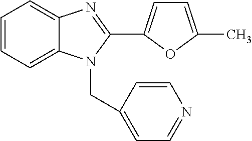 [SMILES: Cc1ccc(-c2nc3ccccc3n2Cc2ccncc2)o1] |
| 25 | 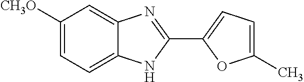 |
| 25a | 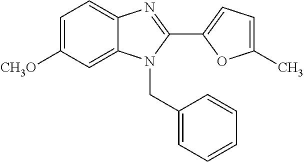 |
| 25b | 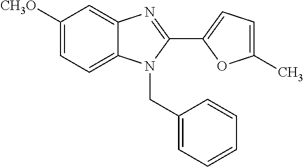 |
| 26a | 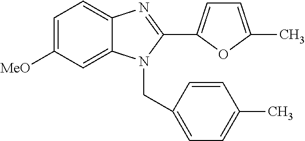 [SMILES: COc1ccc2nc(-c3ccc(C)o3)n(Cc3ccc(C)cc3)c2c1] |
| 26b | 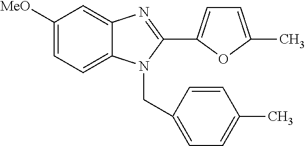 [SMILES: COc1ccc2c(c1)nc(-c1ccc(C)o1)n2Cc1ccc(C)cc1] |
| 27a | 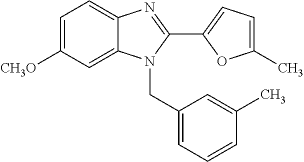 |
| 27b | 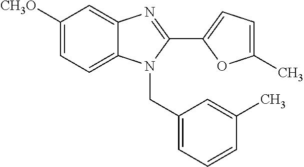 |
| 28a | 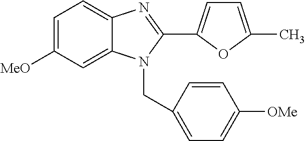 [SMILES: COc1ccc(Cn2c(-c3ccc(C)o3)nc3ccc(OC)cc32)cc1] |
| 28b | 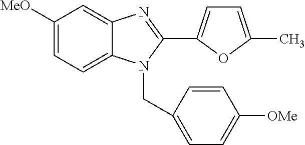 [SMILES: COc1ccc(Cn2c(-c3ccc(C)o3)nc3cc(OC)ccc32)cc1] |
| 29 | 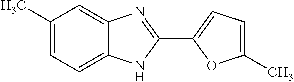 |
| 29a | 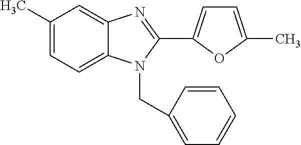 |
| 30 | 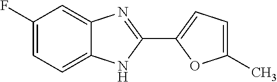 [SMILES: Cc1ccc(-c2nc3cc(F)ccc3[nH]2)o1] |
| 30a | 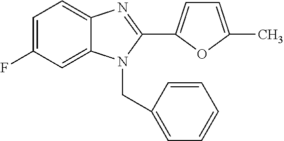 [SMILES: Cc1ccc(-c2nc3ccc(F)cc3n2Cc2ccccc2)o1] |
| 30b | 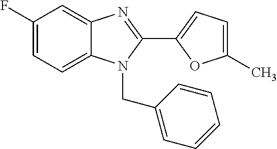 [SMILES: Cc1ccc(-c2nc3cc(F)ccc3n2Cc2ccccc2)o1] |
| 31a | 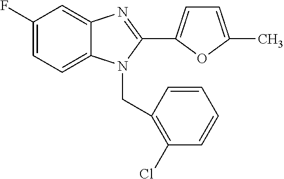 [SMILES: Cc1ccc(-c2nc3cc(F)ccc3n2Cc2ccccc2Cl)o1] |
| 31b | 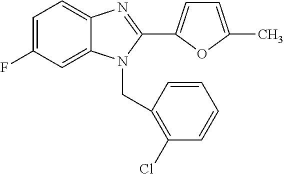 [SMILES: Cc1ccc(-c2nc3ccc(F)cc3n2Cc2ccccc2Cl)o1] |
| 32a | 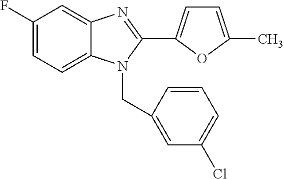 [SMILES: Cc1ccc(-c2nc3cc(F)ccc3n2Cc2cccc(Cl)c2)o1] |
| 32b | 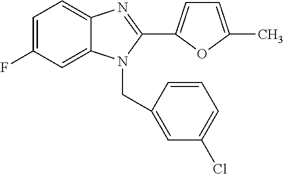 [SMILES: Cc1ccc(-c2nc3ccc(F)cc3n2Cc2cccc(Cl)c2)o1] |
| 33a | 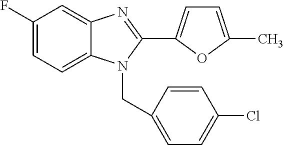 [SMILES: Cc1ccc(-c2nc3cc(F)ccc3n2Cc2ccc(Cl)cc2)o1] |
| 33b | 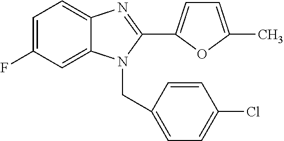 [SMILES: Cc1ccc(-c2nc3ccc(F)cc3n2Cc2ccc(Cl)cc2)o1] |
| 34a | 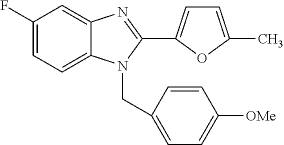 [SMILES: COc1ccc(Cn2c(-c3ccc(C)o3)nc3cc(F)ccc32)cc1] |
| 34b | 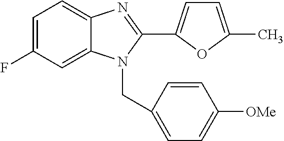 [SMILES: COc1ccc(Cn2c(-c3ccc(C)o3)nc3ccc(F)cc32)cc1] |
| 35a | 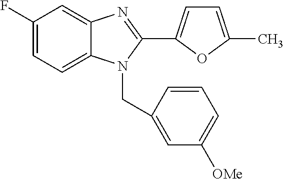 [SMILES: COc1cccc(Cn2c(-c3ccc(C)o3)nc3cc(F)ccc32)c1] |
| 35b | 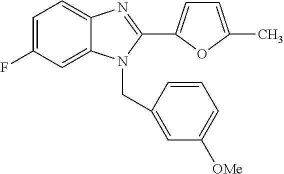 [SMILES: COc1cccc(Cn2c(-c3ccc(C)o3)nc3ccc(F)cc32)c1] |
| 36a | 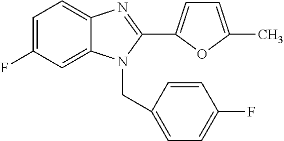 [SMILES: Cc1ccc(-c2nc3ccc(F)cc3n2Cc2ccc(F)cc2)o1] |
| 36b | 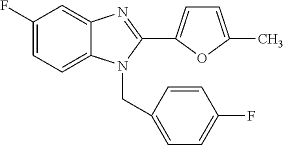 [SMILES: Cc1ccc(-c2nc3cc(F)ccc3n2Cc2ccc(F)cc2)o1] |
| 37a | 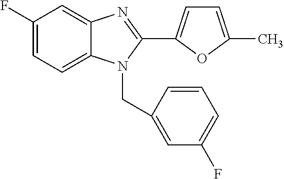 [SMILES: Cc1ccc(-c2nc3cc(F)ccc3n2Cc2cccc(F)c2)o1] |
| 37b | 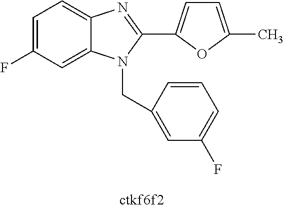 [SMILES: Cc1ccc(-c2nc3ccc(F)cc3n2Cc2cccc(F)c2)o1] |
| 38a | 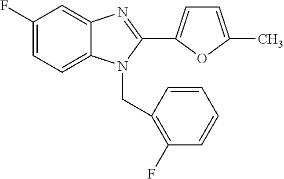 [SMILES: Cc1ccc(-c2nc3cc(F)ccc3n2Cc2ccccc2F)o1] |
| 38b | 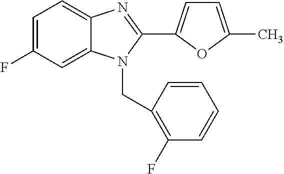 [SMILES: Cc1ccc(-c2nc3ccc(F)cc3n2Cc2ccccc2F)o1] |
| 39a | 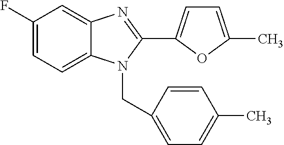 [SMILES: Cc1ccc(Cn2c(-c3ccc(C)o3)nc3cc(F)ccc32)cc1] |
| 39b | 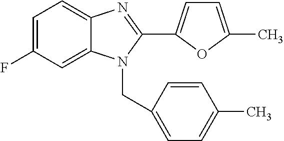 [SMILES: Cc1ccc(Cn2c(-c3ccc(C)o3)nc3ccc(F)cc32)cc1] |
| 40a | 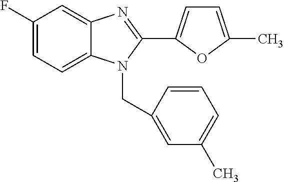 [SMILES: Cc1cccc(Cn2c(-c3ccc(C)o3)nc3cc(F)ccc32)c1] |
| 40b | 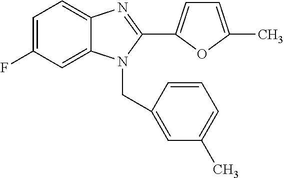 [SMILES: Cc1cccc(Cn2c(-c3ccc(C)o3)nc3ccc(F)cc32)c1] |
| 41a | 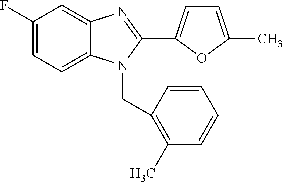 [SMILES: Cc1ccc(-c2nc3cc(F)ccc3n2Cc2ccccc2C)o1] |
| 41b | 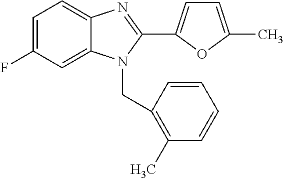 [SMILES: Cc1ccc(-c2nc3ccc(F)cc3n2Cc2ccccc2C)o1] |
| 42 | 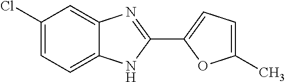 [SMILES: Cc1ccc(-c2nc3cc(Cl)ccc3[nH]2)o1] |
| 42a | 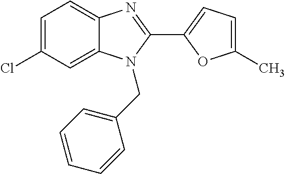 [SMILES: Cc1ccc(-c2nc3ccc(Cl)cc3n2Cc2ccccc2)o1] |
| 42b | 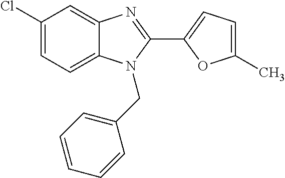 [SMILES: Cc1ccc(-c2nc3cc(Cl)ccc3n2Cc2ccccc2)o1] |
| 43a | 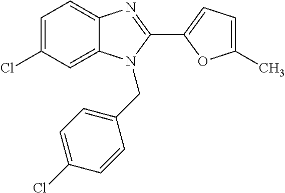 [SMILES: Cc1ccc(-c2nc3ccc(Cl)cc3n2Cc2ccc(Cl)cc2)o1] |
| 43b | 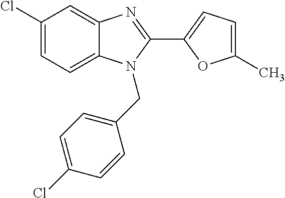 [SMILES: Cc1ccc(-c2nc3cc(Cl)ccc3n2Cc2ccc(Cl)cc2)o1] |
| 44a | 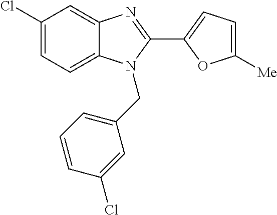 [SMILES: Cc1ccc(-c2nc3cc(Cl)ccc3n2Cc2cccc(Cl)c2)o1] |
| 44b | 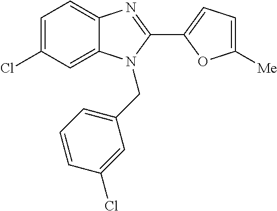 [SMILES: Cc1ccc(-c2nc3ccc(Cl)cc3n2Cc2cccc(Cl)c2)o1] |
| 45a | 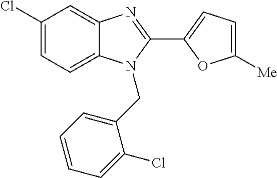 [SMILES: Cc1ccc(-c2nc3cc(Cl)ccc3n2Cc2ccccc2Cl)o1] |
| 45b | 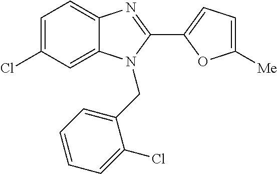 [SMILES: Cc1ccc(-c2nc3ccc(Cl)cc3n2Cc2ccccc2Cl)o1] |
| 46a | 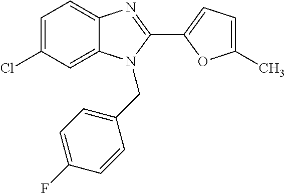 [SMILES: Cc1ccc(-c2nc3ccc(Cl)cc3n2Cc2ccc(F)cc2)o1] |
| 46b | 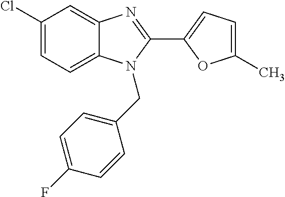 [SMILES: Cc1ccc(-c2nc3cc(Cl)ccc3n2Cc2ccc(F)cc2)o1] |
| 47a | 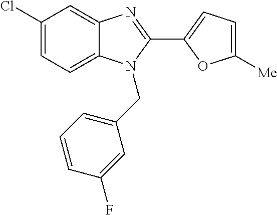 [SMILES: Cc1ccc(-c2nc3cc(Cl)ccc3n2Cc2cccc(F)c2)o1] |
| 47b | 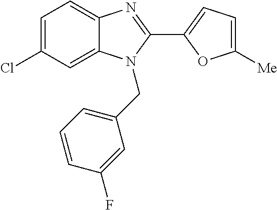 [SMILES: Cc1ccc(-c2nc3ccc(Cl)cc3n2Cc2cccc(F)c2)o1] |
| 48a | 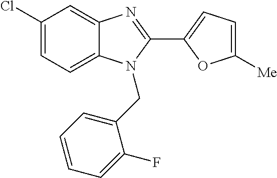 [SMILES: Cc1ccc(-c2nc3cc(Cl)ccc3n2Cc2ccccc2F)o1] |
| 48b | 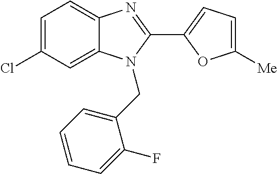 [SMILES: Cc1ccc(-c2nc3ccc(Cl)cc3n2Cc2ccccc2F)o1] |
| 49a | 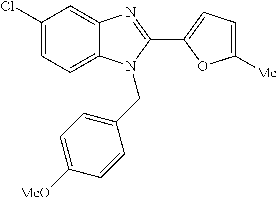 [SMILES: COc1ccc(Cn2c(-c3ccc(C)o3)nc3cc(Cl)ccc32)cc1] |
| 49b | 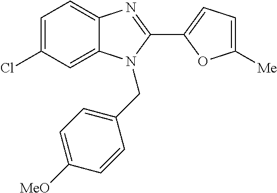 [SMILES: COc1ccc(Cn2c(-c3ccc(C)o3)nc3ccc(Cl)cc32)cc1] |
| 50a | 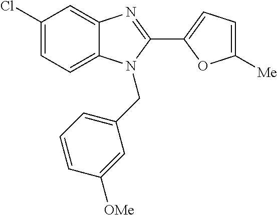 [SMILES: COc1cccc(Cn2c(-c3ccc(C)o3)nc3cc(Cl)ccc32)c1] |
| 50b | 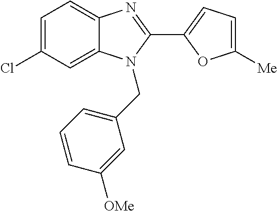 [SMILES: COc1cccc(Cn2c(-c3ccc(C)o3)nc3ccc(Cl)cc32)c1] |
| 51a | 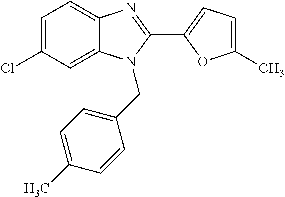 [SMILES: Cc1ccc(Cn2c(-c3ccc(C)o3)nc3ccc(Cl)cc32)cc1] |
| 51b | 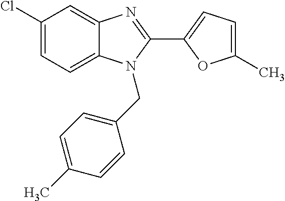 [SMILES: Cc1ccc(Cn2c(-c3ccc(C)o3)nc3cc(Cl)ccc32)cc1] |
| 52a | 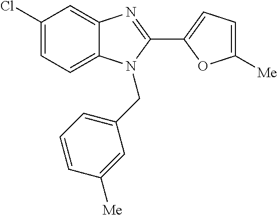 [SMILES: Cc1cccc(Cn2c(-c3ccc(C)o3)nc3cc(Cl)ccc32)c1] |
| 52b | 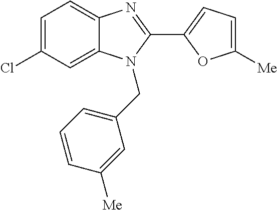 [SMILES: Cc1cccc(Cn2c(-c3ccc(C)o3)nc3ccc(Cl)cc32)c1] |
| 53a | 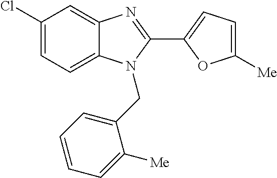 [SMILES: Cc1ccc(-c2nc3cc(Cl)ccc3n2Cc2ccccc2C)o1] |
| 53b | 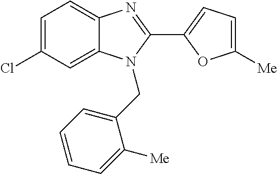 [SMILES: Cc1ccc(-c2nc3ccc(Cl)cc3n2Cc2ccccc2C)o1] |
| 54 | 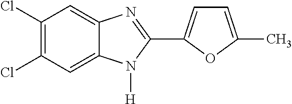 [SMILES: Cc1ccc(-c2nc3cc(Cl)c(Cl)cc3[nH]2)o1] |
| 54a | 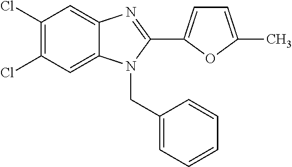 [SMILES: Cc1ccc(-c2nc3cc(Cl)c(Cl)cc3n2Cc2ccccc2)o1] |
| 55 | 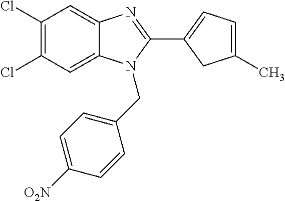 [SMILES: CC1=CC=C(c2nc3cc(Cl)c(Cl)cc3n2Cc2ccc([N+](=O)[O-])cc2)C1] |
| 56 | 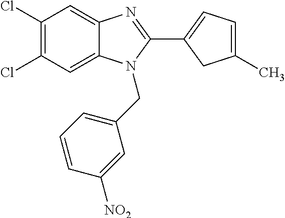 [SMILES: CC1=CC=C(c2nc3cc(Cl)c(Cl)cc3n2Cc2cccc([N+](=O)[O-])c2)C1] |
| 57 | 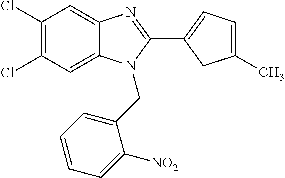 [SMILES: CC1=CC=C(c2nc3cc(Cl)c(Cl)cc3n2Cc2ccccc2[N+](=O)[O-])C1] |
| 58 | 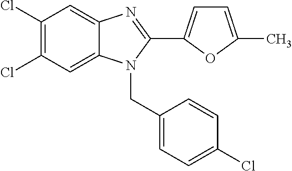 [SMILES: Cc1ccc(-c2nc3cc(Cl)c(Cl)cc3n2Cc2ccc(Cl)cc2)o1] |
| 59 | 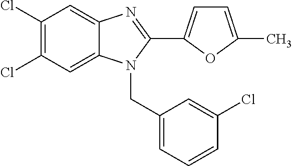 [SMILES: Cc1ccc(-c2nc3cc(Cl)c(Cl)cc3n2Cc2cccc(Cl)c2)o1] |
| 60 | 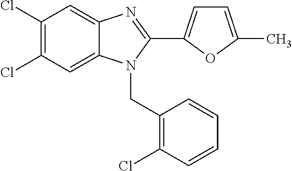 [SMILES: Cc1ccc(-c2nc3cc(Cl)c(Cl)cc3n2Cc2ccccc2Cl)o1] |
| 61 | 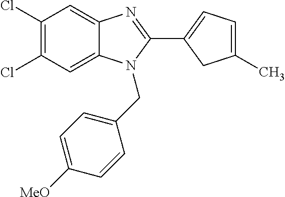 [SMILES: COc1ccc(Cn2c(C3=CC=C(C)C3)nc3cc(Cl)c(Cl)cc32)cc1] |
| 62 | 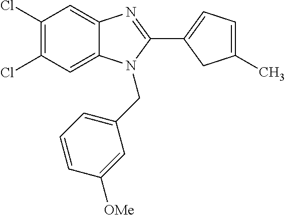 [SMILES: COc1cccc(Cn2c(C3=CC=C(C)C3)nc3cc(Cl)c(Cl)cc32)c1] |
| 63 | 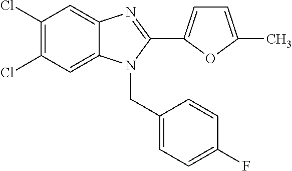 [SMILES: Cc1ccc(-c2nc3cc(Cl)c(Cl)cc3n2Cc2ccc(F)cc2)o1] |
| 64 | 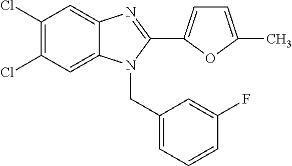 [SMILES: Cc1ccc(-c2nc3cc(Cl)c(Cl)cc3n2Cc2cccc(F)c2)o1] |
| 65 | 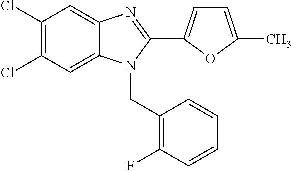 [SMILES: Cc1ccc(-c2nc3cc(Cl)c(Cl)cc3n2Cc2ccccc2F)o1] |
| 66 | 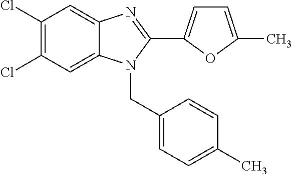 [SMILES: Cc1ccc(Cn2c(-c3ccc(C)o3)nc3cc(Cl)c(Cl)cc32)cc1] |
| 67 | 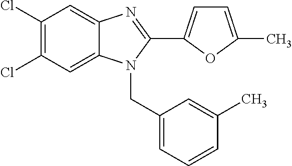 [SMILES: Cc1cccc(Cn2c(-c3ccc(C)o3)nc3cc(Cl)c(Cl)cc32)c1] |
| 68 | 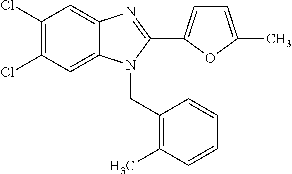 [SMILES: Cc1ccc(-c2nc3cc(Cl)c(Cl)cc3n2Cc2ccccc2C)o1] |
| 69 | 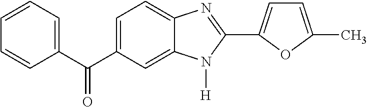 [SMILES: Cc1ccc(-c2nc3ccc(C(=O)c4ccccc4)cc3[nH]2)o1] |
| 69a | 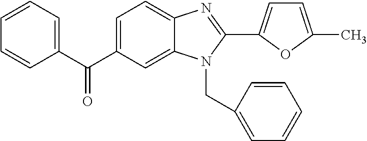 [SMILES: Cc1ccc(-c2nc3ccc(C(=O)c4ccccc4)cc3n2Cc2ccccc2)o1] |
| 70 | 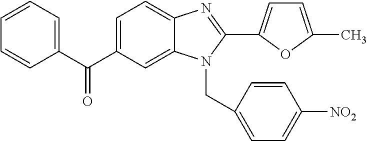 [SMILES: Cc1ccc(-c2nc3ccc(C(=O)c4ccccc4)cc3n2Cc2ccc([N+](=O)[O-])cc2)o1] |
| 71 | 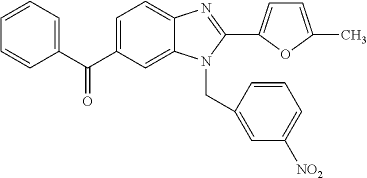 [SMILES: Cc1ccc(-c2nc3ccc(C(=O)c4ccccc4)cc3n2Cc2cccc([N+](=O)[O-])c2)o1] |
| 72 | 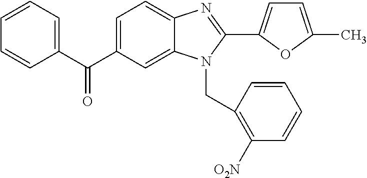 [SMILES: Cc1ccc(-c2nc3ccc(C(=O)c4ccccc4)cc3n2Cc2ccccc2[N+](=O)[O-])o1] |
| 73 | 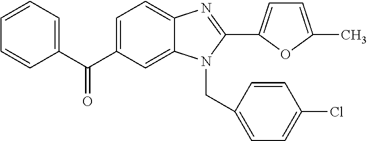 [SMILES: Cc1ccc(-c2nc3ccc(C(=O)c4ccccc4)cc3n2Cc2ccc(Cl)cc2)o1] |
| 74 | 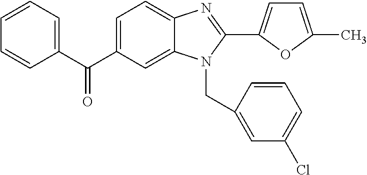 [SMILES: Cc1ccc(-c2nc3ccc(C(=O)c4ccccc4)cc3n2Cc2cccc(Cl)c2)o1] |
| 75 | 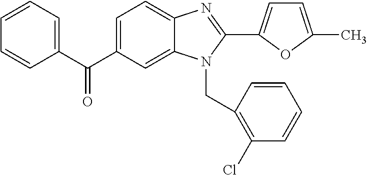 [SMILES: Cc1ccc(-c2nc3ccc(C(=O)c4ccccc4)cc3n2Cc2ccccc2Cl)o1] |
| 76 | 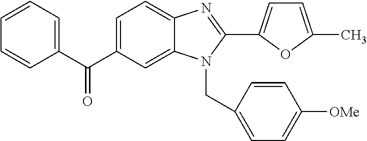 [SMILES: COc1ccc(Cn2c(-c3ccc(C)o3)nc3ccc(C(=O)c4ccccc4)cc32)cc1] |
| 77 | 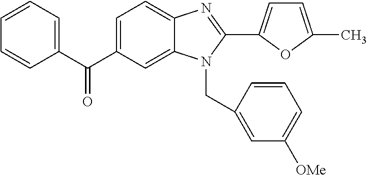 [SMILES: COc1cccc(Cn2c(-c3ccc(C)o3)nc3ccc(C(=O)c4ccccc4)cc32)c1] |
| 78 | 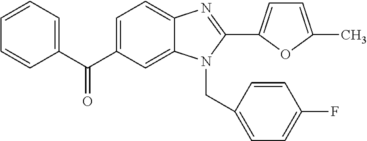 [SMILES: Cc1ccc(-c2nc3ccc(C(=O)c4ccccc4)cc3n2Cc2ccc(F)cc2)o1] |
| 79 | 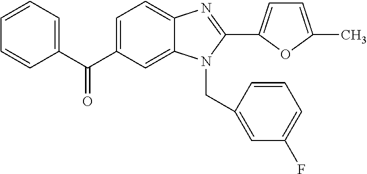 [SMILES: Cc1ccc(-c2nc3ccc(C(=O)c4ccccc4)cc3n2Cc2cccc(F)c2)o1] |
| 80 | 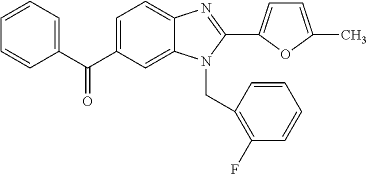 [SMILES: Cc1ccc(-c2nc3ccc(C(=O)c4ccccc4)cc3n2Cc2ccccc2F)o1] |
| 81 | 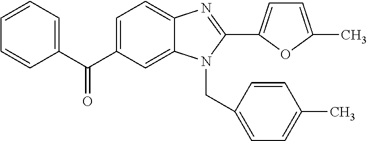 [SMILES: Cc1ccc(Cn2c(-c3ccc(C)o3)nc3ccc(C(=O)c4ccccc4)cc32)cc1] |
| 82 | 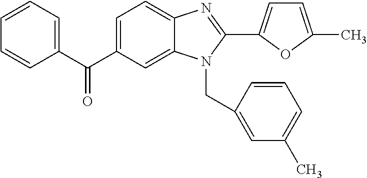 [SMILES: Cc1cccc(Cn2c(-c3ccc(C)o3)nc3ccc(C(=O)c4ccccc4)cc32)c1] |
| 83 | 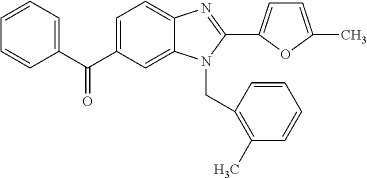 [SMILES: Cc1ccc(-c2nc3ccc(C(=O)c4ccccc4)cc3n2Cc2ccccc2C)o1] |
| 84 | 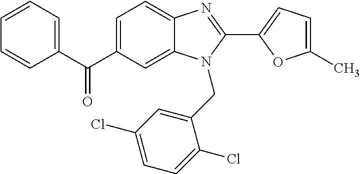 [SMILES: Cc1ccc(-c2nc3ccc(C(=O)c4ccccc4)cc3n2Cc2cc(Cl)ccc2Cl)o1] |
| 85 | 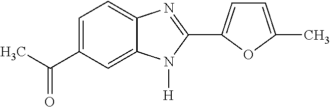 [SMILES: CC(=O)c1ccc2nc(-c3ccc(C)o3)[nH]c2c1] |
| 85a | 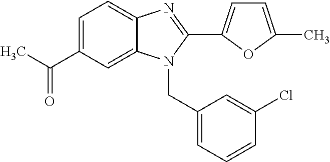 [SMILES: CC(=O)c1ccc2nc(-c3ccc(C)o3)n(Cc3cccc(Cl)c3)c2c1] |
| 85b | 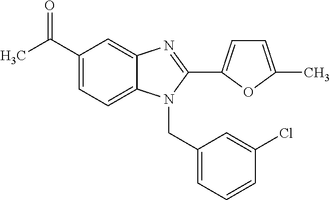 [SMILES: CC(=O)c1ccc2c(c1)nc(-c1ccc(C)o1)n2Cc1cccc(Cl)c1] |
| 86a | 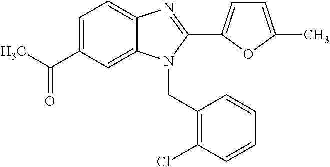 [SMILES: CC(=O)c1ccc2nc(-c3ccc(C)o3)n(Cc3ccccc3Cl)c2c1] |
| 86b | 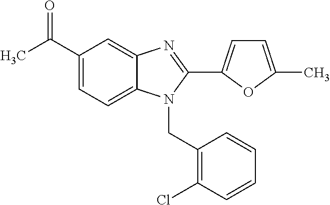 [SMILES: CC(=O)c1ccc2c(c1)nc(-c1ccc(C)o1)n2Cc1ccccc1Cl] |
| 87a | 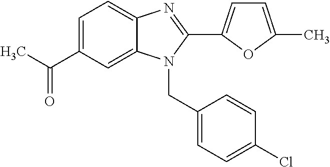 [SMILES: CC(=O)c1ccc2nc(-c3ccc(C)o3)n(Cc3ccc(Cl)cc3)c2c1] |
| 87b | 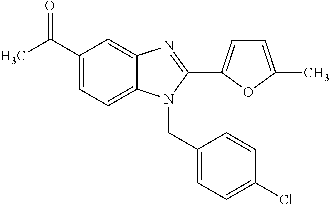 [SMILES: CC(=O)c1ccc2c(c1)nc(-c1ccc(C)o1)n2Cc1ccc(Cl)cc1] |
| 88a | 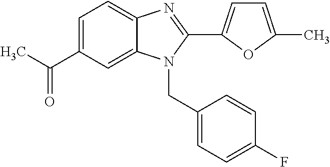 [SMILES: CC(=O)c1ccc2nc(-c3ccc(C)o3)n(Cc3ccc(F)cc3)c2c1] |
| 88b | 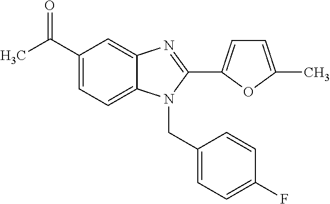 [SMILES: CC(=O)c1ccc2c(c1)nc(-c1ccc(C)o1)n2Cc1ccc(F)cc1] |
| 89a | 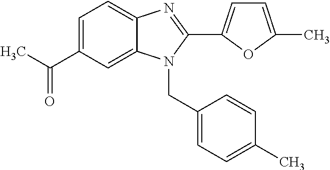 [SMILES: CC(=O)c1ccc2nc(-c3ccc(C)o3)n(Cc3ccc(C)cc3)c2c1] |
| 89b | 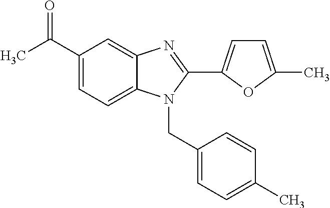 [SMILES: CC(=O)c1ccc2c(c1)nc(-c1ccc(C)o1)n2Cc1ccc(C)cc1] |
| 90 | 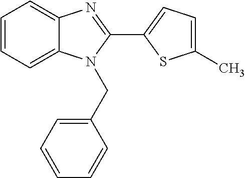 [SMILES: Cc1ccc(-c2nc3ccccc3n2Cc2ccccc2)s1] |
| 91 | 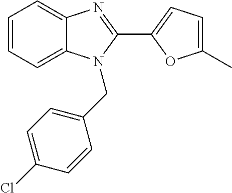 [SMILES: Cc1ccc(-c2nc3ccccc3n2Cc2ccc(Cl)cc2)o1] |
| 92 | 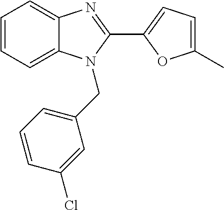 [SMILES: Cc1ccc(-c2nc3ccccc3n2Cc2cccc(Cl)c2)o1] |
| 93 | 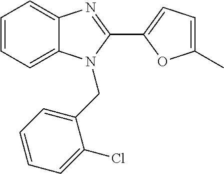 [SMILES: Cc1ccc(-c2nc3ccccc3n2Cc2ccccc2Cl)o1] |
| 94 | 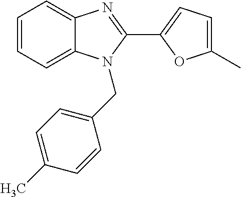 [SMILES: Cc1ccc(Cn2c(-c3ccc(C)o3)nc3ccccc32)cc1] |
| 95 | 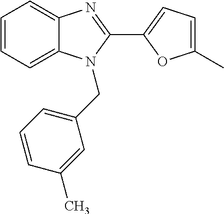 [SMILES: Cc1cccc(Cn2c(-c3ccc(C)o3)nc3ccccc32)c1] |
| 96 | 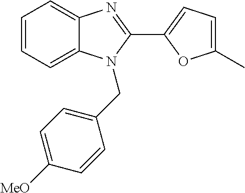 [SMILES: COc1ccc(Cn2c(-c3ccc(C)o3)nc3ccccc32)cc1] |
| 97 | 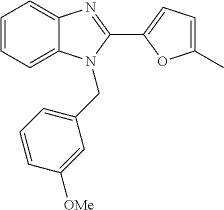 [SMILES: COc1cccc(Cn2c(-c3ccc(C)o3)nc3ccccc32)c1] |
| 98 | 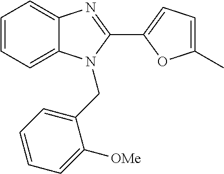 [SMILES: COc1ccccc1Cn1c(-c2ccc(C)o2)nc2ccccc21] |
| 99 | 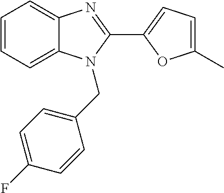 [SMILES: Cc1ccc(-c2nc3ccccc3n2Cc2ccc(F)cc2)o1] |
| 100 | 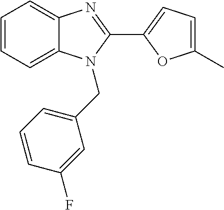 [SMILES: Cc1ccc(-c2nc3ccccc3n2Cc2cccc(F)c2)o1] |
| 101 | 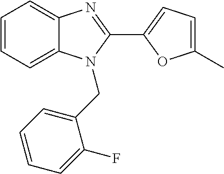 [SMILES: Cc1ccc(-c2nc3ccccc3n2Cc2ccccc2F)o1] |
| 102a | 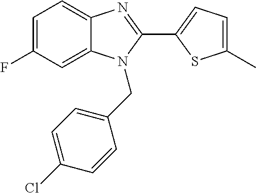 [SMILES: Cc1ccc(-c2nc3ccc(F)cc3n2Cc2ccc(Cl)cc2)s1] |
| 102b | 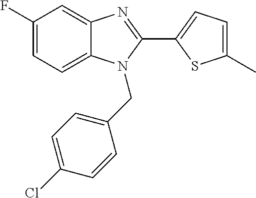 [SMILES: Cc1ccc(-c2nc3cc(F)ccc3n2Cc2ccc(Cl)cc2)s1] |
| 103a | 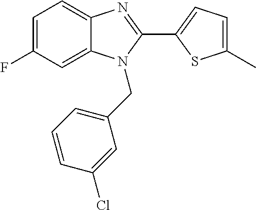 [SMILES: Cc1ccc(-c2nc3ccc(F)cc3n2Cc2cccc(Cl)c2)s1] |
| 103b | 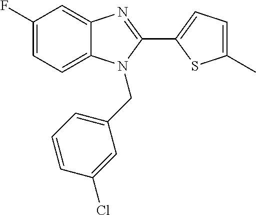 [SMILES: Cc1ccc(-c2nc3cc(F)ccc3n2Cc2cccc(Cl)c2)s1] |
| 104b | 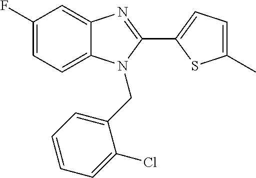 [SMILES: Cc1ccc(-c2nc3cc(F)ccc3n2Cc2ccccc2Cl)s1] |
| 105a | 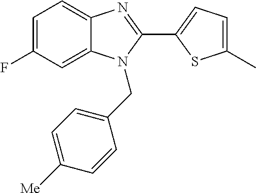 [SMILES: Cc1ccc(Cn2c(-c3ccc(C)s3)nc3ccc(F)cc32)cc1] |
| 105b | 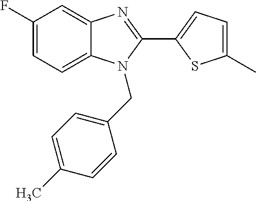 [SMILES: Cc1ccc(Cn2c(-c3ccc(C)s3)nc3cc(F)ccc32)cc1] |
| 106a | 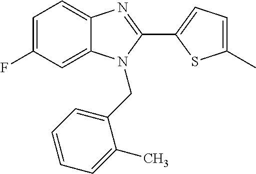 [SMILES: Cc1ccc(-c2nc3ccc(F)cc3n2Cc2ccccc2C)s1] |
| 106b | 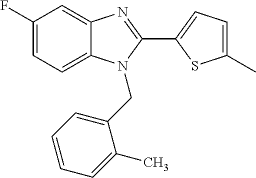 [SMILES: Cc1ccc(-c2nc3cc(F)ccc3n2Cc2ccccc2C)s1] |
| 107a | 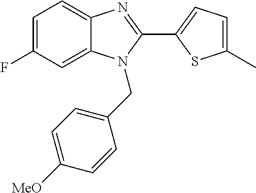 [SMILES: COc1ccc(Cn2c(-c3ccc(C)s3)nc3ccc(F)cc32)cc1] |
| 107b | 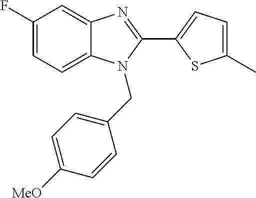 [SMILES: COc1ccc(Cn2c(-c3ccc(C)s3)nc3cc(F)ccc32)cc1] |
| 108a | 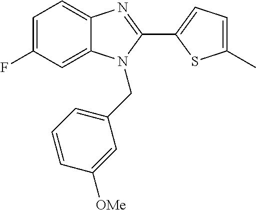 [SMILES: COc1cccc(Cn2c(-c3ccc(C)s3)nc3ccc(F)cc32)c1] |
| 108b | 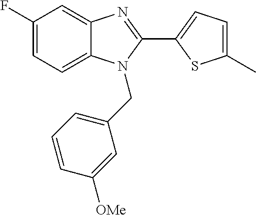 [SMILES: COc1cccc(Cn2c(-c3ccc(C)s3)nc3cc(F)ccc32)c1] |
| 109a | 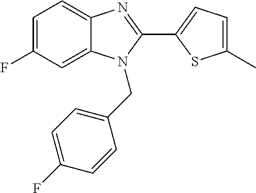 [SMILES: Cc1ccc(-c2nc3ccc(F)cc3n2Cc2ccc(F)cc2)s1] |
| 109b | 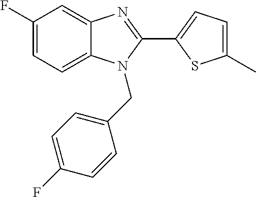 [SMILES: Cc1ccc(-c2nc3cc(F)ccc3n2Cc2ccc(F)cc2)s1] |
| 110a | 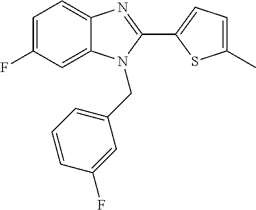 [SMILES: Cc1ccc(-c2nc3ccc(F)cc3n2Cc2cccc(F)c2)s1] |
| 110b | 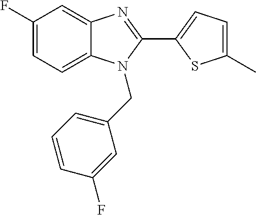 [SMILES: Cc1ccc(-c2nc3cc(F)ccc3n2Cc2cccc(F)c2)s1] |
| 111a | 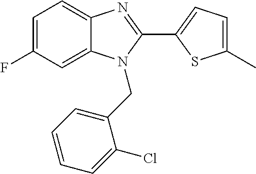 [SMILES: Cc1ccc(-c2nc3ccc(F)cc3n2Cc2ccccc2Cl)s1] |
| 111b | 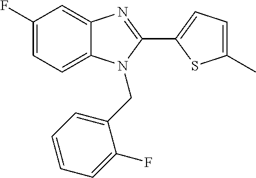 [SMILES: Cc1ccc(-c2nc3cc(F)ccc3n2Cc2ccccc2F)s1] |
| 112a | 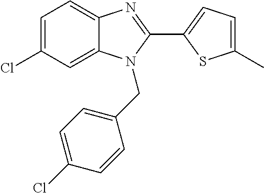 [SMILES: Cc1ccc(-c2nc3ccc(Cl)cc3n2Cc2ccc(Cl)cc2)s1] |
| 112b | 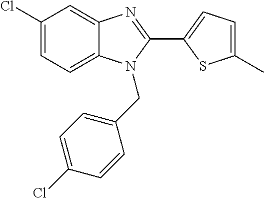 [SMILES: Cc1ccc(-c2nc3cc(Cl)ccc3n2Cc2ccc(Cl)cc2)s1] |
| 113a | 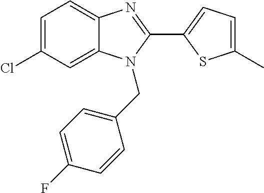 [SMILES: Cc1ccc(-c2nc3ccc(Cl)cc3n2Cc2ccc(F)cc2)s1] |
| 113b | 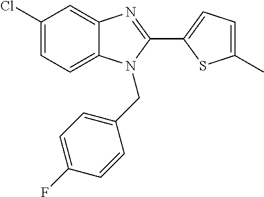 [SMILES: Cc1ccc(-c2nc3cc(Cl)ccc3n2Cc2ccc(F)cc2)s1] |

In other aspect, the invention provides a method for inhibiting platelet aggregation, comprising administering an effective amount of compound or a pharmaceutical composition of the invention to a subject in need of such treatment. Preferably, the platelet aggregation is caused by collagen.

In a further aspect, the invention provides a method for the prevention and/or treatment of thrombogenic diseases, comprising administering an effective amount of compound or a pharmaceutical composition of the invention to a subject in need of such treatment.

The compounds and pharmaceutical composition of the invention provide efficacy as antithrombotic agents by their ability to exhibit platelet aggregation inhibitory activity and antithrombotic activity. The compounds and pharmaceutical compositions of the invention can be used for preventing or treating diseases or conditions associated with platelet aggregation and/or platelet activation. Accordingly, they can be used for preventing or treating thrombosis and related disorders, such as venous thrombosis, established peripheral arterial disease, thrombophlebitis, arterial embolism, coronary and cerebral arterial thrombosis, unstable angina, myocardial infarction, stroke, cerebral embolism, renal embolism, pulmonary embolism and other embolism- or thrombosis-related afflictions produced by but not limited to procedural or surgical interventions. This invention further provides methods for the prevention of embolism or thrombosis during percutaneous coronary interventions, placement of coronary stents, coronary angioplasty, coronary endarectomy, carotid endarectomy, or due to platelet-aggregation complications related to atherosclerosis, inflammation, exposure of blood to artificial devices, drug effects.

The compounds and pharmaceutical compositions of the invention are useful as anti-thrombotic agents, and are thus useful in the treatment or prevention of unstable angina, coronary angioplasty (PTCA) and myocardial infarction.

The compounds and pharmaceutical compositions of the invention are useful in the treatment or prevention of primary arterial thrombotic complications of atherosclerosis such as thrombotic stroke, peripheral vascular disease, and myocardial infarction without thrombolysis.

The compounds and pharmaceutical compositions of the invention are useful for the treatment or prevention of arterial thrombotic complications due to interventions in atherosclerotic disease such as angioplasty, endarterectomy, stent placement, coronary and other vascular graft surgery.

The compounds and pharmaceutical compositions of the invention are useful for the treatment or prevention of thrombotic complications of surgical or mechanical damage such as tissue salvage following surgical or accidental trauma, reconstructive surgery including skin flaps, and “reductive” surgery such as breast reduction.

The compounds and pharmaceutical compositions of the invention are useful for the prevention of mechanically-induced platelet activation in vivo, for example, caused by cardiopulmonary bypass, which results in temporary platelet dysfunction (prevention of microthromboembolism). The compounds and pharmaceutical compositions of the invention are useful for prevention of mechanically-induced platelet activation in vitro. For example, the compounds are useful in the preservation of blood products, e.g. platelet concentrates, prevention of shunt occlusion such as renal dialysis and plasmapheresis, and thrombosis secondary to vascular damage/inflammation such as vasculitis, arteritis, glomerulonephritis and organ graft rejection.

The compounds and pharmaceutical compositions of the invention are useful in disorders with a diffuse thrombotic/platelet consumption component such as disseminated intravascular coagulation, thrombotic thrombocytopenic purpura, hemolytic uremic syndrome, heparin-induced thrombocytopenia and pre-eclampsia/eclampsia.

The compounds and pharmaceutical compositions of the invention are useful for the treatment or prevention of venous thrombosis such as deep vein thrombosis, veno-occlusive disease, hematological conditions such as thrombocythemia and polycythemia, and migraine

The compounds and pharmaceutical compositions of the invention are useful in treating a mammal to alleviate the pathological effects of atherosclerosis and arteriosclerosis, acute MI, chronic stable angina, unstable angina, transient ischemic attacks and strokes, peripheral vascular disease, arterial thrombosis, preeclampsia, embolism, restenosis or abrupt closure following angioplasty, carotid endarterectomy, and anastomosis of vascular grafts.

The compounds and pharmaceutical compositions of the invention are useful in treating chronic or acute states of hyper-aggregability, such as disseminated intravascular coagulation (DIC), septicemia, surgical or infectious shock, post-operative and post-partum trauma, cardiopulmonary bypass surgery, incompatible blood transfusion, abruptio placenta, thrombotic thrombocytopenic purpura (TTP), and immune diseases, are likely to be responsive to such treatment.

The compounds and pharmaceutical compositions of the invention are useful in treating diseases or conditions associated with platelet activation and/or aggregation produced by the contact of blood with an artificial device. In one embodiment, the artificial device is a paracorporeal artificial lung and an extracorporeal membrane oxygenation device. In another embodiment, the artificial device is an internal implantable artificial heart. In another embodiment, the artificial device is an apheresis instrument used to remove or isolate a specific component of the blood, and returning the remaining blood components to the donor. In yet another embodiment, the artificial device is a hemodialysis instrument.

The compounds of the present invention are useful in vitro to inhibit the aggregation of platelets in blood and blood products, e.g. for storage, or for ex vivo manipulations such as in diagnostic or research use. In such applications, the compounds are administered to the blood or blood product.

In another preferred embodiment, the compounds and pharmaceutical compositions of the invention are useful as adjunctive therapy in the prevention or treatment of thrombotic disorders, such as coronary arterial thrombosis during the management of unstable angina, coronary angioplasty and acute myocardial infarction, i.e. perithrombolysis. The compounds and pharmaceutical compositions of the invention are administered in combination with other antiplatelet and/or anticoagulant drugs such as heparin, aspirin, GP IIb/IIIa antagonists, or thrombin inhibitors.

Applications of compounds and pharmaceutical compositions of the invention include prevention of platelet thrombosis, thromboembolism and reocclusion during and after thrombolytic therapy and prevention of platelet thrombosis, thromboembolism and reocclusion after angioplasty of coronary and other arteries and after coronary artery bypass procedures. In addition, other applications of compounds such as asthma and inflammatory disease also can be development.

The compounds and compositions of the invention can be administered by any suitable route, locally or systemically, including, for example, by parenteral administration. Parenteral administration can include, for example, intramuscular, intravenous, subcutaneous, or intraperitoneal injection. Topical administration can include, for example, creams, gels, ointments or aerosols. Respiratory administration can include, for example, inhalation or intranasal drops.

Scheme 1 below depicts routes that can be followed to synthesize certain benzimidazole compounds of the present invention.

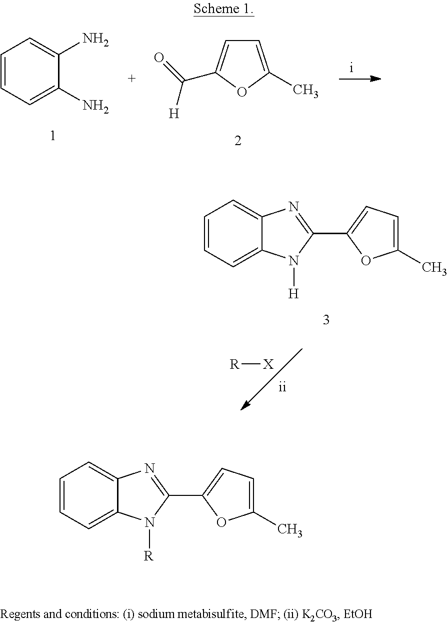

### EXAMPLES

### Example 1

### Synthesis of 1-Methyl-2-(5-methyl-2-furyl)benzimidazole

### (a) Synthesis of 2-(5-methyl-2-furyl)benzimidazole

1,2-Phenylenediamine (1.3 g, 10 mmol), was condensed with sodium metabisulfite adduct of 5-methyl-2-furaldehyde (1.3 g, 10 mmol), in DMF (10 mL). The reaction mixture was then heated under reflux for 6 hrs, and after cooling was added with water (120 mL). The precipitate was filtered and was recrystallized from ethanol to affored 2-(5-methyl-2-furyl)benzimidazole.

¹H-NMR (200 MHz, DMSO-d₆): 2.40 (3H, s, 5′-CH₃), 6.33 (1H, d, J=3.2 Hz, H-4′), 7.06 (1H, d, J=3.2 Hz, H-3′), 7.05-7.18 (2H, m, H-4, 7), 7.48-7.52 (2H, m, H-5, 6), 12.77 (1H, s, NH)

### (b) Synthesis of 1-methyl-2-(5-methyl-2-furyl)benzimidazole

2-(5-Methyl-2-furyl)benzimidazole (3, 0.2 g, 1.0 mmol) was mixed with potassium carbonate (0.7 g, 5 mmol) in 95% ethanol (50 ml) and heated to boiling. Methyl iodide (5 mmol) was added dropwise with vigorous stirring of the mixture. After complete addition of the Methyl iodide and reflux for about 4 hrs. The reaction mixture was cooled and poured into water. The aqueous solution was extracted with dichloromethane, and the dichloromethane extracts were washed with water and dried over sodium sulfate. Evaporation of the solvent gave an oil that was purified by chromatography on a silica gel column using dichloromethane as eluent affording the expected products 1-methyl-2-(5-methyl-2-furyl)benzimidazole

¹H-NMR (200 MHz, CDCl₃): 2.45 (3H, s, 5′-CH₃), 3.99 (3H, s, N—CH₃), 6.20 (1H, d, J=3.2 Hz, H-4′), 7.05 (1H, d, J=3.2 Hz, H-3′), 7.25-7.32 (3H, m, H-4, 5, 6), 7.76-7.80 (1H, m, H-7)

### Example 2: 1-Ethyl-2-(5-methyl-2-furyl)benzimidazole

1-Ethyl-2-(5-methyl-2-furyl)benzimidazole was prepared as in Example 1(b)

¹H-NMR (200 MHz, CDCl₃): 1.50 (3H, t, J=7.1 Hz, H-2″), 2.42 (3H, s, 5′-CH₃), 4.48 (2H, q, J=7.1 Hz, N—CH₂—), 6.19 (1H, d, J=3.2 Hz, H-4′), 7.08 (1H, d, J=3.2 Hz, H-3′), 7.26-7.37 (3H, m, H-4, 5, 6), 7.75-7.85 (1H, m, H-7)

### Example 3: 1-Propyl-2-(5-methyl-2-furyl)benzimidazole

1-Propyl-2-(5-methyl-2-furyl)benzimidazole was prepared as in Example 1(b).

¹H-NMR (200 MHz, CDCl₃): 1.00 (3H, t, J=7.4 Hz, H-3″), 1.86-1.97 (2H, m, H-2″), 2.44 (3H, s, 5′-CH₃), 4.40 (2H, t, J=7.4 Hz, N—CH₂—), 6.19 (1H, d, J=3.2 Hz, H-4′), 7.06 (1H, d, J=3.2 Hz, H-3′), 7.24-7.38 (3H, m, H-4, 5, 6), 7.76-7.86 (1H, m, H-7)

### Example 4: 1-Butyl-2-(5-methyl-2-furyl)benzimidazole

1-Butyl-2-(5-methyl-2-furyl)benzimidazole was prepared as in Example 1(b).

¹H-NMR (200 MHz, CDCl₃): 1.00 (3H, t, J=7.4 Hz, H-4″), 1.39-1.50 (2H, m, H-3″), 1.80-1.94 (2H, m, H-2″), 2.43 (3H, s, 5′-CH₃), 4.43 (2H, t, J=7.4 Hz, N—CH₂—), 6.20 (1H, d, J=3.2 Hz, H-4′), 7.10 (1H, d, J=3.2 Hz, H-3′), 7.26-7.36 (3H, m, H-4, 5, 6), 7.75-7.80 (1H, m, H-7)

### Example 5: 1-Pentyl-2-(5-methyl-2-furyl)benzimidazole

1-Pentyl-2-(5-methyl-2-furyl)benzimidazole was prepared as in Example 1(b)

¹H-NMR (200 MHz, CDCl₃): 0.91 (3H, t, J=7.4 Hz, H-5″), 1.23-1.41 (4H, m, H-3″, 4″), 1.80-1.90 (2H, m, H-2″), 2.45 (3H, s, 5′-CH₃), 4.43 (2H, t, J=7.4 Hz, N—CH₂—), 6.21 (1H, d, J=3.2 Hz, H-4′), 7.14 (1H, d, J=3.2 Hz, H-3′), 7.27-7.36 (3H, m, H-4, 5, 6), 7.75-7.79 (1H, m, H-7)

### Example 6: 1-Benzyl-2-(5-methyl-2-furyl)benzimidazole (nstpbp5185)

1-Benzyl-2-(5-methyl-2-furyl)benzimidazole was prepared as in Example 1(b)

¹H-NMR (200 MHz, CDCl₃): 2.38 (3H, s, 5′-CH₃), 5.64 (2H, s, N—CH₂—), 6.12 (1H, d, J=3.3 Hz, H-4′), 6.88 (1H, d, J=3.3 Hz, H-3′), 7.12-7.15 (2H, m, H-2″, 6″), 7.23-7.33 (6H, m, H-4, 5, 6, 3″, 4″, 5″), 7.84 (1H, d, J=8.2 Hz, H-7)

### Example 7: 1-(o-Methylbenzyl)-2-(5-methyl-2-furyl)benzimidazole

1-(o-Methylbenzyl)-2-(5-methyl-2-furyl)benzimidazole was prepared as in Example 1(b)

¹H-NMR (200 MHz, CDCl₃): 2.25 (3H, s, 2″-CH₃), 2.39 (3H, s, 5′-CH₃), 5.44 (2H, s, N—CH₂—), 6.03 (1H, d, J=3.3 Hz, H-4′), 6.45 (1H, d, J=7.5 Hz, H-6″), 6.77 (1H, d, J=3.3 Hz, H-3′), 6.92 (1H, dd, J=7.0, 7.0 Hz, H-5″), 7.12-7.24 (5H, m, H-4, 5, 6, 3″, 4″), 7.84 (1H, d, J=8.2 Hz, H-7)

### Example 8: 1-(m-Methylbenzyl)-2-(5-methyl-2-furyl)benzimidazole

1-(m-Methylbenzyl)-2-(5-methyl-2-furyl)benzimidazole was prepared as in Example 1(b)

¹H-NMR (200 MHz, CDCl₃): 2.28 (3H, s, 3″-CH₃), 2.39 (3H, s, 5′-CH₃), 5.62 (2H, s, N—CH₂—), 6.14 (1H, d, J=3.3 Hz, H-4′), 6.95-7.44 (8H, m, H-4, 5, 6, 3′, 2″, 4″, 5″, 6″), 7.87 (1H, d, J=8.2 Hz, H-7)

### Example 9:1-(p-Methylbenzyl)-2-(5-methyl-2-furyl)benzimidazole

1-(p-Methylbenzyl)-2-(5-methyl-2-furyl)benzimidazole was prepared as in Example 1(b)

¹H-NMR (200 MHz, CDCl₃): 2.28 (3H, s, 4″-CH₃), 2.39 (3H, s, 5′-CH₃), 5.63 (2H, s, N—CH₂—), 6.14 (1H, d, J=3.3 Hz, H-4′), 7.02-7.09 (5H, m, H-3′, 2″, 3″, 5″, 6″), 7.23-7.34 (3H, m, H-4, 5, 6), 7.85 (1H, d, J=8.2 Hz, H-7)

### Example 10: 1-(o-Methoxylbenzyl)-2-(5-methyl-2-furyl)benzimidazole

1-(o-Methoxylbenzyl)-2-(5-methyl-2-furyl)benzimidazole was prepared as in Example 1(b)

¹H-NMR (200 MHz, CDCl₃): 2.38 (3H, s, 5′-CH₃), 3.71 (3H, s, 2″-OCH₃), 5.57 (2H, s, N—CH₂—), 6.13 (1H, d, J=3.3 Hz, H-4′), 6.68-6.81 (2H, m, H-3″, 5″), 6.92 (1H, d, J=3.3 Hz, H-3′), 6.94-7.42 (5H, m, H-4, 5, 6, 4″, 6″), 7.80 (1H, d, J=8.2 Hz, H-7)

### Example 11: 1-(m-Methoxylbenzyl)-2-(5-methyl-2-furyl)benzimidazole

1-(m-Methoxylbenzyl)-2-(5-methyl-2-furyl)benzimidazole was prepared as in Example 1(b)

¹H-NMR (200 MHz, CDCl₃): 2.39 (3H, s, 5′-CH₃), 3.71 (3H, s, 3″-OCH₃), 5.62 (2H, s, N—CH₂—), 6.13 (1H, d, J=3.3 Hz, H-4′), 6.69-6.82 (3H, m, H-2″, 4″, 6″), 6.91 (1H, d, J=3.3 Hz, H-3′), 6.95-7.44 (4H, m, H-4, 5, 6, 5″), 7.83 (1H, d, J=8.2 Hz, H-7)

### Example 12: 1-(p-Methoxylbenzyl)-2-(5-methyl-2-furyl)benzimidazole

1-(p-Methoxylbenzyl)-2-(5-methyl-2-furyl)benzimidazole was prepared as in Example 1(b)

¹H-NMR (200 MHz, CDCl₃): 2.38 (3H, s, 5′-CH₃), 3.72 (3H, s, 4″-OCH₃), 5.56 (2H, s, N—CH₂—), 6.13 (1H, d, J=3.3 Hz, H-4′), 6.78-6.83 (4H, m, H-2″, 3″, 5″, 6″), 6.94 (1H, d, J=3.3 Hz, H-3′), 7.23-7.27 (3H, m, H-4, 5, 6), 7.77 (1H, d, J=8.2 Hz, H-7)

### Example 13: 1-(o-Fluorobenzyl)-2-(5-methyl-2-furyl)benzimidazole

1-(o-Fluorobenzyl)-2-(5-methyl-2-furyl)benzimidazole was prepared as in Example 1(b)

¹H-NMR (200 MHz, CDCl₃): 2.35 (3H, s, 5′-CH₃), 5.72 (2H, s, N—CH₂—), 6.13 (1H, d, J=3.3 Hz, H-4′), 6.75 (1H, dd, J=7.0, 7.0 Hz, H-5″), 6.92-7.44 (7H, m, H-4, 5, 6, 3′, 3″, 4″, 6″), 7.82 (1H, d, J=8.2 Hz, H-7)

### Example 14: 1-(m-Fluorobenzyl)-2-(5-methyl-2-furyl)benzimidazole

1-(m-Fluorobenzyl)-2-(5-methyl-2-furyl)benzimidazole was prepared as in Example 1(b)

¹H-NMR (200 MHz, CDCl₃): 2.37 (3H, s, 5′-CH₃), 5.66 (2H, s, N—CH₂—), 6.15 (1H, d, J=3.3 Hz, H-4′), 6.82-6.95 (4H, m, H-3′, 2″, 4″, 6″), 7.21-7.27 (4H, m, H-4, 5, 6, 5″), 7.83 (1H, d, J=8.2 Hz, H-7)

### Example 15: 1-(p-Fluorobenzyl)-2-(5-methyl-2-furyl)benzimidazole

1-(p-Fluorobenzyl)-2-(5-methyl-2-furyl)benzimidazole was prepared as in Example 1(b)

¹H-NMR (200 MHz, CDCl₃): 2.38 (3H, s, 5′-CH₃), 5.62 (2H, s, N—CH₂—), 6.14 (1H, d, J=3.3 Hz, H-4′), 6.94-7.15 (5H, m, H-3′, 2″, 3″, 5″, 6″), 7.26-7.53 (3H, m, H-4, 5, 6), 7.81 (1H, d, J=8.2 Hz, H-7)

### Example 16: 1-(o-Chlorobenzyl)-2-(5-methyl-2-furyl)benzimidazole

1-(o-Chlorobenzyl)-2-(5-methyl-2-furyl)benzimidazole was prepared as in Example 1(b)

¹H-NMR (200 MHz, CDCl₃): 2.30 (3H, s, 5′-CH₃), 5.69 (2H, s, N—CH₂—), 6.08 (1H, d, J=3.3 Hz, H-4′), 6.55 (1H, d, J=7.0 Hz, H-6″), 6.83 (1H, d, J=3.3 Hz, H-3′), 7.03 (1H, dd, J=7.0, 7.0 Hz, H-5″), 7.23-7.52 (5H, m, H-4, 5, 6, 3″, 4″), 7.84 (1H, d, J=8.2 Hz, H-7)

### Example 17: 1-(m-Chlorobenzyl)-2-(5-methyl-2-furyl)benzimidazole

1-(m-Chlorobenzyl)-2-(5-methyl-2-furyl)benzimidazole was prepared as in Example 1(b)

¹H-NMR (200 MHz, CDCl₃): 2.37 (3H, s, 5′-CH₃), 5.62 (2H, s, N—CH₂—), 6.14 (1H, d, J=3.3 Hz, H-4′), 6.98 (1H, d, J=3.3 Hz, H-3′), 7.17-7.27 (7H, m, H-4, 5, 6, 2″, 4″, 5″, 6″), 7.83 (1H, d, J=8.2 Hz, H-7)

### Example 18: 1-(p-Chlorobenzyl)-2-(5-methyl-2-furyl)benzimidazole

1-(p-Chlorobenzyl)-2-(5-methyl-2-furyl)benzimidazole was prepared as in Example 1(b)

¹H-NMR (200 MHz, CDCl₃): 2.37 (3H, s, 5′-CH₃), 5.62 (2H, s, N—CH₂—), 6.15 (1H, d, J=3.3 Hz, H-4′), 7.01 (1H, d, J=3.3 Hz, H-3′), 7.07 (2H, d, J=8.3 Hz, H-2″, 6″), 7.25-7.33 (5H, m, H-4, 5, 6, 3″, 5″), 7.83 (1H, d, J=8.2 Hz, H-7)

### Example 19: 1-(o-Nitrobenzyl)-2-(5-methyl-2-furyl)benzimidazole

1-(o-Nitrobenzyl)-2-(5-methyl-2-furyl)benzimidazole was prepared as in Example 1(b)

¹H-NMR (200 MHz, CDCl₃): 2.37 (3H, s, 5′-CH₃), 5.77 (2H, s, N—CH₂—), 6.09 (1H, d, J=3.3 Hz, H-4′), 6.90 (1H, d, J=3.3 Hz, H-3′), 7.23-7.29 (5H, m, H-4, 5, 6, 4″, 6″), 7.42-7.46 (2H, m, H-7, 5″), 7.88 (1H, d, J=8.2 Hz, H-3″)

### Example 20: 1-(m-Nitrobenzyl)-2-(5-methyl-2-furyl)benzimidazole

1-(m-Nitrobenzyl)-2-(5-methyl-2-furyl)benzimidazole was prepared as in Example 1(b)

¹H-NMR (200 MHz, CDCl₃): 2.36 (3H, s, 5′-CH₃), 5.76 (2H, s, N—CH₂—), 6.15 (1H, d, J=3.3 Hz, H-4′), 7.03 (1H, d, J=3.3 Hz, H-3′), 7.28-7.49 (5H, m, H-4, 5, 6, 5″, 6″), 7.83 (1H, d, J=8.2 Hz, H-7), 8.11-8.16 (2H, m, H-2″, 4″)

### Example 21: 1-(p-Nitrobenzyl)-2-(5-methyl-2-furyl)benzimidazole (24)

1-(p-Nitrobenzyl)-2-(5-methyl-2-furyl)benzimidazole was prepared as in Example 1(b)

¹H-NMR (200 MHz, CDCl₃): 2.28 (3H, s, 5′-CH₃), 5.68 (2H, s, N—CH₂—), 6.07 (1H, d, J=3.3 Hz, H-4′), 6.92 (1H, d, J=3.3 Hz, H-3′), 7.17-7.29 (5H, m, H-4, 5, 6, 2″, 6″), 7.77 (1H, d, J=8.2 Hz, H-7), 8.08 (2H, d, J=8.6 Hz, H-3″, 5″)

### Example 22: 1-(2-Picoyl)-2-(5-methyl-2-furyl)benzimidazole

1-(2-Picoyl)-2-(5-methyl-2-furyl)benzimidazole was prepared as in Example 1(b)

¹H-NMR (200 MHz, CDCl₃): 2.33 (3H, s, 5′-CH₃), 5.76 (2H, s, N—CH₂—), 6.11 (1H, d, J=3.3 Hz, H-4′), 6.77 (1H, d, J=7.9 Hz, H-6″), 6.93 (1H, d, J=3.3 Hz, H-3′), 7.25-7.32 (4H, m, H-4, 5, 6, 4″), 7.51-7.52 (1H, m, H-5″), 7.83 (1H, d, J=8.2 Hz, H-7), 8.62 (1H, d, J=5.0 Hz, H-3″)

### Example 23: 1-(3-Picoyl)-2-(5-methyl-2-furyl)benzimidazole

1-(3-Picoyl)-2-(5-methyl-2-furyl)benzimidazole was prepared as in Example 1(b)

¹H-NMR (200 MHz, DMSO-d₆): 2.30 (3H, s, 5′-CH₃), 5.78 (2H, s, N—CH₂—), 6.41 (1H, d, J=3.3 Hz, H-4′), 7.04 (1H, d, J=3.3 Hz, H-3′), 7.26-7.70 (6H, m, H-4, 5, 6, 7, 5″, 6″), 8.32-8.38 (2H, m, H-2″, 4″)

### Example 24: 1-(4-Picoyl)-2-(5-methyl-2-furyl)benzimidazole

1-(4-Picoyl)-2-(5-methyl-2-furyl)benzimidazole was prepared as in Example 1(b)

¹H-NMR (200 MHz, CDCl₃): 2.32 (3H, s, 5′-CH₃), 5.64 (2H, s, N—CH₂—), 6.12 (1H, d, J=3.3 Hz, H-4′), 6.92 (1H, d, J=3.3 Hz, H-3′), 7.02 (2H, d, J=5.1 Hz, H-2″, 6″′), 7.22-7.34 (3H, m, H-4, 5, 6), 7.82 (1H, d, J=8.2 Hz, H-7), 8.53 (2H, d, J=5.1 Hz, H-3″, 5″)

### Example 25: Synthesis of 1-benzyl-6-methoxy-2-(5-methyl-2-furyl)benzimidazole (Example 25a) and 1-benzyl-5-methoxy-2-(5-methyl-2-furyl)benzimidazole (Example 25b)

5-Methoxy-2-(5-methyl-2-furyl)benzimidazole (0.5 g, 2.4 mmol) (Example 25) was similarly prepared as in Example 1 and used as the starting material to react with benzyl chloride to obtain Example 25a (0.2 g, 26.2%) and Example 25b (0.2 g, 26.2%) by column chromatography (Silica, ethyl acetate:hexane=1:3).

### Example 25a

¹H-NMR (CDCl₃) δ: 2.32 (3H, s, 5′-CH₃), 3.75 (3H, s, 4″-OCH₃), 5.54 (2H, s, —CH₂), 6.05 (1H, dd, J=3.2, 0.9 Hz, H-4′), 6.67 (1H, d, J=2.3 Hz, H-7), 6.74 (1H, d, J=3.4 Hz H-3′), 6.88 (1H, dd, J=8.8, 2.4 Hz, H-5), 7.07-7.28 (5H, m, H-2″, 3″, 4″, 5″, 6″), 7.67 (1H, d, J=8.8 Hz, H-4)

### Example 25b

¹H-NMR (CDCl₃) δ: 2.33 (3H, s, 5′-CH₃), 3.82 (3H, s, —OCH₃), 5.56 (2H, s, —CH₂), 6.07 (1H, dd, J=3.3, 0.9 Hz, H-4′), 6.82 (2H, dd, J=8.8, 2.7 Hz, H-6, 3′), 7.07-7.30 (7H, m, H-4, 7, 2″, 3″, 4″, 5″, 6″)

### Example 26: Synthesis of 6-methoxy-1-(4-methylbenzyl)-2-(5-methyl-2-furyl)-1H-benzimidazole (Example 26a) and 5-methoxy-1-(4-methylbenzyl)-2-(5-methyl-2-furyl)-1H-benzimidazole (Example 26b)

6-Methoxy-1-(4-methylbenzyl)-2-(5-methyl-2-furyl) benzimidazole (Example 26a) and 5-methoxy-1-(4-methylbenzyl)-2-(5-methyl-2-furyl)-benzimidazole (Example 26b) was prepared as in Example 25 at a yield of Example 26a (0.26 g, 32.6%) and Example 26b (0.19 g, 23.3%).

### Example 26a

¹H-NMR (CDCl₃) δ: 2.27 (3H, s, 4″-CH₃), 2.33 (3H, s, 5′-CH₃), 3.76 (3H, s, 5-OCH₃), 5.50 (2H, s, —CH₂), 6.06 (1H, d, J=3.3 Hz, H-4′), 6.68 (1H, d, J=2.3 Hz, H-7), 6.73 (1H, d, J=3.3 Hz, H-3′), 6.88 (1H, dd, J=8.8, 2.3 Hz, H-5), 6.99 (2H, d, J=8.2 Hz, H-2″, 6″), 7.08 (2H, d, J=8.2 Hz, H-3″, 5″), 7.66 (1H, d, J=8.8 Hz, H-4)

### Example 26b

MS (m/z): 332 (M⁺⁾

IR(KBr)vₘₐₓ: 1612, 1566, 1490, 1438 cm⁻¹ (C═C, C═N)

UV, λₘₐₓ (MeOH) nm (log ε): 327 (4.33)

¹H-NMR (CDCl₃) δ: 2.30 (3H, s, 4″-CH₃), 2.38 (3H, s, 5′-CH₃), 3.86 (3H, s, 5-OCH₃), 5.57 (2H, s, —CH₂), 6.12 (1H, d, J=3.3 Hz, H-4′), 6.87 (1H, dd, J=8.8, 2.4 Hz, H-6), 6.90 (1H, d, J=3.3 Hz, H-3′), 7.02 (2H, d, J=8.0 Hz, H-2″, 6″), 7.10 (2H, d, J=8.0 Hz, H-3″, 5″), 7.13 (1H, d, J=8.8 Hz, H-7), 7.30 (1H, d, J=2.3 Hz, H-4)

### Example 27: Synthesis of 6-methoxy-1-(3-methylbenzyl)-2-(5-methyl-2-furyl)benzimidazole (Example 27a) and 5-methoxy-1-(3-methylbenzyl)-2-(5-methyl-2-furyl)benzimidazole (Example 27b)

6-Methoxy-1-(3-methylbenzyl)-2-(5-methyl-2-furyl) benzimidazole (Example 27a) and 5-methoxy-1-(3-methylbenzyl)-2-(5-methyl-2-furyl)-benzimidazole (Example 27b) was prepared as in Example 25 at a yield of Example 27a (0.22 g, 28.1%) and Example 27b (0.18 g, 22.2%).

### Example 27a

¹H-NMR (CDCl₃) δ: 2.24 (3H, s, 3″-CH₃), 2.33 (3H, s, 5′-CH₃), 3.76 (3H, s, 5-OCH₃), 5.49 (2H, s, —CH₂), 6.05 (1H, d, J=3.3 Hz, H-4′), 6.68 (1H, s, H-2″), 6.71 (1H, d, J=3.3 Hz, H-3′), 6.86-7.23 (5H, m, H-5, 7, 4″, 5″, 6″), 7.10 (2H, d, J=8.0 Hz, H-2″), 7.67 (1H, d, J=8.8 Hz, H-4)

### Example 27b

¹H-NMR (CDCl₃) δ: 2.23 (3H, s, 3″-CH₃), 2.34 (3H, s, 5′-CH₃), 3.82 (3H, s, 5-OCH₃), 5.53 (2H, s, —CH₂), 6.08 (1H, dd, J=3.4, 0.9 Hz, H-4′), 6.80 (1H, d, J=3.3 Hz, H-3′), 6.84 (1H, dd, J=8.8, 2.4 Hz, H-6), 6.90-7.15 (5H, m, H-7, 2″, 4″, 5″, 6″), 7.27 (1H, d, J=2.3 Hz, H-4)

### Example 28: Synthesis of 1-(4-methoxylbenzyl)-6-methoxy-2-(5-methyl-2-furyl)benzimidazole (Example 28a) and 1-(4-methoxylbenzyl)-5-methoxy-2-(5-methyl-2-furyl)benzimidazole (Example 28b)

1-(4-Methoxylbenzyl)-6-methoxy-2-(5-methyl-2-furyl) benzimidazole (Example 28a) and 1-(3-methoxylbenzyl)-5-methoxy-2-(5-methyl-2-furyl)-benzimidazole (Example 28b) was prepared as in Example 25 at a yield of Example 28a (0.26 g, 32.6%) and Example 28b (0.10 g, 11.5%).

### Example 28a

¹H-NMR (CDCl₃) δ: 2.34 (3H, s, 5′-CH₃), 3.72 (3H, s, 4″-OCH₃), 3.76 (3H, s, 5-OCH₃), 5.48 (2H, s, —CH₂), 6.07 (1H, d, J=3.3 Hz, H-4′), 6.69 (1H, d, J=2.3 Hz, H-7), 6.77-6.82 (3H, m, H-3′, 3″, 5″), 6.87 (1H, dd, J=8.8, 2.4 Hz, H-5), 7.04 (2H, d, J=9.6 Hz, H-2″, 6″), 7.65 (1H, d, J=8.8 Hz, H-4).

### Example 28b

¹H-NMR (CDCl₃) δ: 2.35 (3H, s, 5′-CH₃), 3.71 (3H, s, 4″-OCH₃), 3.81 (3H, s, 5-OCH₃), 5.50 (2H, s, —CH₂), 6.08 (1H, dd, J=3.3, 1.0 Hz, H-4′), 6.69 (1H, d, J=2.3 Hz, H-4), 6.75-6.85 (4H, m, H-3′, 3″, 5″, 6), 7.03 (2H, d, J=8.8 Hz, H-2″, 6″), 7.10 (1H, d, J=8.8 Hz, H-7), 7.24 (1H, d, J=2.3 Hz H-4).

### Example 29: Synthesis of 1-benzyl-5-methyl-2-(5-methyl-2-furyl)benzimidazole

1-Benzyl-5-methyl-2-(5-methyl-2-furyl)benzimidazole (Example 29a) was prepared from 5-methyl-2-(5-methyl-2-furyl)benzimidazole (Example 29) as in Example 25 at a yield of 41.6% (0.33 g).

### Example 29

¹H-NMR (CDCl₃) δ: 2.22 (3H, s, 5′-CH₃), 2.41 (3H, s, 5-CH₃), 6.03 (1H, d, J=3.3 Hz, H-4′), 7.00 (1H, d, J=2.9 Hz, H-3′), 7.02 (1H, d, J=9.1 Hz, H-6), 7.34 (1H, s, H-4), 7.46 (1H, d, J=8.2 Hz, H-7), 8.45 (1H, s, NH)

### Example 29a

¹H-NMR (CDCl₃) δ: 2.34 (3H, s, 5′-CH₃), 2.44 (3H, s, 5-CH₃), 5.59 (2H, s, —CH₂), 6.08 (1H, dd, J=3.3, 0.9 Hz, H-4′), 6.88 (1H, d, J=3.4 Hz H-3′), 7.00-7.29 (7H, m, H-6, 7, 2″, 3″, 4″, 5″, 6″), 7.59 (1H, s, H-4)

### Example 30: Synthesis of 1-benzyl-6-fluoro-2-(5-methyl-2-furyl)benzimidazole (Example 30a) and 1-benzyl-5-fluoro-2-(5-methyl-2-furyl)benzimidazole (Example 30b)

1-Benzyl-6-fluoro-2-(5-methyl-2-furyl)benzimidazole (Example 30a) and 1-benzyl-5-fluoro-2-(5-methyl-2-furyl)benzimidazole (Example 30b) was prepared as in Example 25 at a yield of Example 30a (0.18 g, 24.1%) and Example 30b (0.10 g, 13.2%).

### Example 30a

¹H-NMR (CDCl₃) δ: 2.34 (3H, s, 5′-CH₃), 5.60 (2H, s, —CH₂), 6.10 (1H, d J=2.7 Hz, H-4′), 6.87 (2H, d, J=3.0 Hz, H-3′), 6.95 (1H, dd, J=9.2, 2.3 Hz, H-4), 7.07-7.31 (6H, m, H-7, 2″, 3″, 4″, 5″, 6″), 7.44 (1H, dd, J=9.2, 2.3 Hz, H-5)

### Example 30b

¹H-NMR (CDCl₃) δ: 2.34 (3H, s, 5′-CH₃), 5.56 (2H, s, —CH₂), 6.09 (1H, dd, J=3.3, 0.8 Hz, H-4′), 6.85 (1H, d, J=3.4 Hz, H-3′), 6.88-7.31 (7H, m, H-4, 6, 2″, 3″, 4″, 5″, 6″), 7.69 (1H, dd, J=8.8, 4.8 Hz, H-7)

### Example 31: Synthesis of 5-fluoro-1-(2-chlorobenzyl)-2-(5-methyl-2-furyl)benzimidazole (Example 31a) and 6-fluoro-1-(2-chlorobenzyl)-2-(5-methyl-2-furyl)benzimidazole (Example 31b)

5-Fluoro-1-(2-chlorobenzyl)-2-(5-methyl-2-furyl)benzimidazole (Example 31a) and 6-fluoro-1-(2-chlorobenzyl)-2-(5-methyl-2-furyl)benzimidazole (Example 3 b) was prepared as in Example 25 at a yield of Example 31a (0.19 g, 23.1%) and Example 31b (0.04 g, 5%).

### Example 31a

¹H-NMR (CDCl₃-d₁, 200 MHz) δ(ppm): 2.31 (3H, s, 5′-CH₃), 5.67 (2H, s, —CH₂), 6.08 (1H, d, J=2.84 Hz, H-4′), 6.54 (1H, d, J=7.56 Hz, H-6″), 6.79 (1H, d, J=3.18 Hz, H-3′), 6.91˜7.42 (4H, m, H-6, 7, 4″, 5″), 7.44˜7.50 (2H, m, H-3″, 4)

¹³C-NMR (CDCl₃-d₁, 50 MHz) δ(ppm): 13.73, 46.18, 105.36, 105.84, 108.32, 109.67, 109.87, 111.15, 111.68, 114.00, 126.67, 127.48, 128.96, 129.62, 131.99, 132.26, 133.69, 142.67, 155.09

### Example 31b

¹H-NMR (CDCl₃-d₁, 200 MHz) δ(ppm): 2.30 (3H, s, 5′-CH₃), 5.65 (2H, s, —CH₂), 6.09 (1H, m, H-4′), 6.57 (1H, d, J=6.84 Hz, H-6″), 6.81 (1H, d, J=3.36, H-3′), 6.87˜7.23 (4H, m, H-5, 7, 4″, 5″), 7.43 (1H, dd, J=1.12, 7.90 Hz, H-3″), 7.72 (1H, dd, J=4.78, 8.8 Hz, H-3″)

¹³C-NMR (CDCl₃-d₁, 50 MHz) δ(ppm): 13.69, 46.21, 96.08, 96.63, 108.27, 111.20, 111.70, 113.79, 120.63, 126.66, 127.48, 129.01, 129.66, 132.02, 133.48, 142.58, 154.94

### Example 32: Synthesis of 5-fluoro-1-(3-chlorobenzyl)-2-(5-methyl-2-furyl)benzimidazole (Example 32a) and 6-fluoro-1-(3-chlorobenzyl)-2-(5-methyl-2-furyl)benzimidazole (Example 32b)

5-Fluoro-1-(3-chlorobenzyl)-2-(5-methyl-2-furyl)benzimidazole (Example 32a) and 6-fluoro-1-(3-chlorobenzyl)-2-(5-methyl-2-furyl)benzimidazole (Example 32b) was prepared as in Example 25 at a yield of Example 32a (0.19 g, 23.0%) and Example 32b (0.19 g, 23.0%).

### Example 32a

¹H-NMR (DMSO-d₆, 400 MHz) δ(ppm): 2.35 (3H, s, 5′-CH₃), 5.80 (2H, s, —CH₂), 6.33 (1H, s, H-4′), 7.00˜7.32 (6H, m, H-6, 7, 3′, 2″, 4″, 5″, 6″), 7.47 (1H, d, J=6.84 Hz, H-6), 7.66 (1H, s, H-4)

¹³C-NMR (DMSO-d₆, 100 MHz) δ(ppm): 13.74, 47.43, 154.87, 142.99, 140.12, 133.68, 132.84, 131.10, 127.88, 126.82, 125.32, 114.72, 111.78, 111.68, 111.41, 111.15, 108.97, 105.03, 104.79

### Example 32b

¹H-NMR (DMSO-d₁, 400 MHz) δ(ppm): 2.34 (3H, s, 5′-CH₃), 5.77 (2H, s, —CH₂), 6.30 (1H, d, J=1.99 Hz, H-4′), 7.01˜7.34 (6H, m, H-7, 3′, 2″, 4″, 5″, 6″), 7.59 (1H, m, H-5), 7.66 (1H, dd, J=4.8, 8.8 Hz, H-4)

¹³C-NMR (CDCl₃-d₁, 100 MHz) δ(ppm): 13.69, 47.43, 97.60, 97.88, 108.85, 110.97, 111.22, 114.20, 120.36, 120.46, 125.33, 126.85, 127.86, 131.04, 133.69, 139.53, 140.04, 143.02, 154.59

### Example 33: Synthesis of 6-fluoro-1-(4-chlorobenzyl)-2-(5-methyl-2-furyl) benzimidazole (Example 33a) and 5-fluoro-1-(4-chlorobenzyl)-2-(5-methyl-2-furyl)benzimidazole (Example 33b)

5-Fluoro-1-(3-chlorobenzyl)-2-(5-methyl-2-furyl)benzimidazole (Example 33a) and 6-fluoro-1-(3-chlorobenzyl)-2-(5-methyl-2-furyl)benzimidazole (Example 33b) was prepared as in Example 25 at a yield of Example 33a (0.12 g, 15.0%) and Example 33b (0.13 g, 15.0%).

### Example 33a

¹H-NMR (CDCl₃-d₁, 200 MHz) δ(ppm): (Chart 39-1)

2.34 (3H, s, 5′-CH₃), 5.57 (2H, s, —CH₂), 6.11 (1H, d, J=2.7, H-4′), 6.90 (1H, d, J=3.3, H-3′), 6.89˜7.29 (6H, m, H-4, 5, 2″, 3″, 5″, 6″), 7.44 (1H, dd, J=2.2, 9.3, H-7)

¹³C-NMR (CDCl₃-d₁, 50 MHz) δ(ppm): (Chart 39-2)

13.81, 47.90, 105.26, 105.74, 108.43, 109.81, 110.01, 111.13, 111.65, 114.43, 127.57, 129.17, 132.08, 133.75, 134.65, 142.80, 145.90, 154.95

### Example 33b

¹H-NMR (CDCl₃-d₁, 200 MHz) δ(ppm): (Chart 40-1)

2.33 (3H, s, 5′-CH₃), 5.53 (2H, s, —CH₂), 6.10 (1H, d, J=2.7, H-4′), 6.86 (1H, d, J=3.1, H-3′), 6.89˜7.05 (6H, m, H-6, 7, 2″, 3″, 5″, 6″), 7.69 (1H, dd, J=4.8, 8.8, H-4)

¹³C-NMR (CDCl₃-d₁, 50 MHz) δ(ppm): (Chart 40-2)

13.79, 47.90, 96.15, 96.71, 108.31, 111.06, 111.56, 113.84, 120.46, 120.66, 127.58, 129.19, 133.78, 134.50, 139.47, 142.89, 145.39, 154.70

### Example 34: Synthesis of 5-fluoro-1-(4-methoxybenzyl)-2-(5-methyl-2-furyl)benzimidazole (Example 34a) and 6-fluoro-1-(4-methoxybenzyl)-2-(5-methyl-2-furyl)benzimidazole (Example 34b)

5-Fluoro-1-(4-methoxybenzyl)-2-(5-methyl-2-furyl)benzimidazole (Example 34a) and 6-fluoro-1-(4-methoxybenzyl)-2-(5-methyl-2-furyl)benzimidazole (Example 34b) was prepared as in Example 25 at a yield of Example 34a (0.11 g, 15.0%) and Example 34b (0.19 g, 20.0%).

### Example 34a

¹H-NMR (CDCl₃-d₁, 200 MHz) δ(ppm): 2.37 (3H, s, 5′-CH₃), 3.73 (1H, s, 4″-OCH₃), 5.54 (2H, s, —CH₂), 6.10 (1H, dd, J=0.88, 3.22 Hz, H-4′), 6.78˜7.23 (7H, m, H-6, 7, 3′, 2″, 3″, 5″, 6″), 7.40˜7.46 (1H, m, H-4)

¹³C-NMR (CDCl₃-d₁, 50 MHz) δ(ppm): 13.86, 47.95, 55.27, 105.16, 105.64, 108.30, 110.04, 110.25, 110.89, 111.41, 114.07, 114.32, 127.53, 128.07, 132.30, 142.98, 146.05, 154.82, 159.17

### Example 34b

¹H-NMR (CDCl₃-d₁, 200 MHz) δ(ppm): 2.37 (3H, s, 5′-CH₃), 3.73 (1H, s, 4″-OCH₃), 5.50 (2H, s, —CH₂), 6.09˜6.11 (1H, m, H-4′), 6.78˜7.06 (7H, m, H-5, 7, 3′, 2″, 3″, 5″, 6″), 7.64 (1H, dd, J=4.82, 8.74 Hz, H-4)

¹³C-NMR (CDCl₃-d₁, 50 MHz) δ(ppm): 13.82, 47.99, 55.26, 96.39, 96.94, 108.21, 110.82, 111.32, 113.66, 114.36, 120.32, 120.52, 127.58, 127.89, 139.52, 143.02, 154.60, 159.21

### Example 35: Synthesis of 5-Fluoro-1-(3-methoxybenzyl)-2-(5-methyl-2-furyl)benzimidazole (Example 35a) and 6-Fluoro-1-(3-methoxybenzyl)-2-(5methyl-2-furyl)benzimidazole (Example 35b)

5-Fluoro-1-(3-methoxybenzyl)-2-(5-methyl-2-furyl)benzimidazole (Example 35a) and 6-fluoro-1-(3-methoxybenzyl)-2-(5-methyl-2-furyl)benzimidazole (Example 35b) was prepared as in Example 25 at a yield of Example 35a (0.14 g, 17.0%) and Example 35b (0.10 g, 12.0%).

### Example 35a

¹H-NMR (CDCl₃-d₁, 200 MHz) δ(ppm): 2.35 (3H, s, 5′-CH₃), 3.68 (1H, s, 3″-OCH₃), 5.56 (2H, s, —CH₂), 6.09˜6.11 (1H, m, H-4′), 6.62˜7.23 (7H, m, H-6, 7, 3′, 2″, 4″, 5″, 6″), 7.41 (1H, dd, J=2.4, 9.40 Hz, H-4)

¹³C-NMR (CDCl₃-d₁, 50 MHz) δ(ppm): 13.82, 46.19, 48.35, 55.17, 105.21, 105.69, 108.30, 109.98, 110.19, 110.95, 111.47, 112.17, 112.83, 114.08, 118.40, 120.78, 130.09, 132.37, 137.73, 142.90, 154.87, 160.11

### Example 35b

¹H-NMR (CDCl₃-d₁, 200 MHz) δ(ppm): 2.35 (3H, s, 5′-CH₃), 3.69 (1H, s, 3″-OCH₃), 5.53 (2H, s, —CH₂), 6.09˜6.10 (1H, m, H-4′), 6.63˜7.24 (7H, m, H-5, 7, 3′, 2″, 4″, 5″, 6″), 7.66 (1H, dd, J=4.84, 8.76 Hz, H-4)

¹³C-NMR (CDCl₃-d₁, 50 MHz) δ(ppm): 13.82, 46.19, 48.35, 55.17, 105.21, 105.69, 108.30, 109.98, 110.19, 110.95, 111.47, 112.17, 112.83, 114.08, 118.40, 120.78, 130.09, 132.37, 137.73, 142.90, 154.87, 160.11

### Example 36: Synthesis of 6-Fluoro-1-(4-fluorobenzyl)-2-(5-methyl-2-furyl) benzimidazole (Example 36a) and 5-Fluoro-1-(4-fluorobenzyl)-2-(5-methyl-2-furyl)benzimidazole (Example 36b)

6-Fluoro-1-(4-fluorobenzyl)-2-(5-methyl-2-furyl)benzimidazole (Example 36a) and 5-fluoro-1-(4-fluorobenzyl)-2-(5-methyl-2-furyl)benzimidazole (Example 36b) was prepared as in Example 25 at a yield of Example 36a (0.14 g, 18.0%) and Example 36b (0.18 g, 23.0%).

### Example 36a

¹H-NMR (CDCl₃-d₁, 200 MHz) δ(ppm): (Chart 41-1)

2.35 (3H, s, 5′-CH₃), 5.57 (2H, s, —CH₂), 6.11 (1H, d, J=2.5, H-4′), 6.88˜7.13 (7H, m, H-4, 5, 3′, 2″, 3″, 5″, 6″), 7.43 (1H, dd, J=2.3, 9.3, H-7)

¹³C-NMR (CDCl₃-d₁, 50 MHz) δ(ppm): (Chart 41-2)

13.80, 47.82, 105.25, 105.73, 108.37, 109.84, 110.04, 111.04, 111.56, 114.26, 115.71, 116.14, 127.84, 128.00, 131.92, 132.15, 142.91, 143.72, 145.94, 154.87, 164.71

### Example 36b

¹H-NMR (CDCl₃-d₁, 200 MHz) δ(ppm): (Chart 42-1)

2.34 (3H, s, 5′-CH₃), 5.54 (2H, s, —CH₂), 6.11 (1H, d, J=2.3, H-4′), 6.86˜7.23 (7H, m, H-6, 7, 3′, 2″, 3″, 5″, 6″), 7.68 (1H, dd, J=4.8, 8.8, H-4)

¹³C-NMR (CDCl₃-d₁, 50 MHz) δ(ppm): (Chart 42-2)

13.78, 47.85, 96.21, 96.76, 108.30, 111.02, 111.51, 113.83, 115.74, 116.18, 120.43, 120.63, 127.88, 128.04, 131.75, 135.70, 139.46, 142.93, 154.67

### Example 37: Synthesis of 5-fluoro-1-(3-fluorobenzyl)-2-(5-methyl-2-furyl)benzimidazole (Example 37a) and 6-fluoro-1-(3-fluorobenzyl)-2-(5-methyl-2-furyl)benzimidazole (Example 37b)

5-Fluoro-1-(3-fluorobenzyl)-2-(5-methyl-2-furyl)benzimidazole (Example 37a) and 6-fluoro-1-(3-fluorobenzyl)-2-(5-methyl-2-furyl)benzimidazole (Example 37b) was prepared as in Example 25 at a yield of Example 37a (0.10 g, 12.0%) and Example 37b (0.21 g, 28.0%).

### Example 37a

¹H-NMR (CDCl₃-d₁, 200 MHz) δ(ppm): 2.37 (3H, s, 5′-CH₃), 5.62 (2H, s, —CH₂), 6.15 (1H, s, H-4′), 6.81 (1H, d, J=9.2 Hz, H-3′), 6.89˜6.99 (4H, m, H-2″, 4″, 5″, 6″), 7.13 (1H, dd, J=4.4, 8.39 Hz, H-7), 7.25˜7.31 (1H, m, H-6), 7.47 (1H, dd, J=0.8, 4.6 Hz, H-4)

¹³C-NMR (CDCl₃-d₁, 50 MHz) δ(ppm): 13.79, 18.41, 48.05, 58.39, 105.18, 105.66, 108.49, 109.86, 110.06, 111.24, 111.76, 113.13, 113.58, 114.72, 115.13, 121.79, 130.57, 130.73, 132.03, 142.62, 145.79, 155.09

### Example 37b

¹H-NMR (CDCl₃-d₁, 200 MHz) δ(ppm): 2.38 (3H, s, 5′-CH₃), 5.60 (2H, s, —CH₂), 6.15 (1H, s, H-4′), 6.83˜7.31 (7H, m, H-5, 7, 3′, 2″, 4″, 5″, 6″), 7.29 (1H, d, J=3.8 Hz, H-6), 7.74˜7.76 (1H, m, H-4)

¹³C-NMR (CDCl₃-d₁, 50 MHz) δ(ppm): 13.80, 48.03, 105.24, 105.73, 108.46, 109.82, 110.03, 111.20, 111.72, 113.12, 113.57, 114.57, 114.70, 115.12, 121.78, 130.56, 130.73, 132.08, 138.64, 155.05

### Example 38: Synthesis of 5-fluoro-1-(2-fluorobenzyl)-2-(5-methyl-2-furyl)benzimidazole (Example 38a) and 6-Fluoro-1-(2-fluorobenzyl)-2-(5-methyl-2-furyl)benzimidazole (Example 38b)

5-Fluoro-1-(2-fluorobenzyl)-2-(5-methyl-2-furyl)benzimidazole (Example 37a) and 6-fluoro-1-(2-fluorobenzyl)-2-(5-methyl-2-furyl)benzimidazole (Example 37b) was prepared as in Example 25 at a yield of Example 38a (0.30 g, 37.0%) and Example 38b (0.27 g, 34.0%).

### Example 38a

¹H-NMR (CDCl₃-d₁, 200 MHz) δ(ppm): 2.27 (3H, s, 5′-CH₃), 5.78 (2H, s, —CH₂), 6.25 (1H, s, H-4′), 6.66˜6.73 (1H, m, H-5″), 6.97˜7.23 (5H, m, H-6, 7, 3″, 4″, 6″), 7.40 (1H, d, J=9.34 Hz, H-5), 7.53˜7.59 (1H, m, H-4)

¹³C-NMR (CDCl₃-d₁, 50 MHz) δ(ppm): 13.66, 42.55, 104.67, 105.14, 108.92, 110.94, 111.46, 111.57, 111.77, 114.59, 115.70, 116.12, 124.41, 124.69, 125.16, 128.20, 128.26, 129.97, 130.13, 133.00, 143.25, 143.57, 145.85, 154.77

### Example 38b

¹H-NMR (CDCl₃-d₁, 400 MHz) δ(ppm): 2.37 (3H, s, 5′-CH₃), 5.67 (2H, s, —CH₂), 6.14 (1H, d, J=2.00 Hz, H-4′), 6.76˜6.79 (1H, m, H-5″), 6.91˜7.28 (6H, m, H-5, 7, 3″, 4″, 5″, 6″), 7.22 (1H, dd, J=4.8, 8.8 Hz, H-4)

¹³C-NMR (CDCl₃-d₁, 100 MHz) δ(ppm): 13.62, 42.14, 108.19, 113.64, 120.40, 120.50, 123.16, 123.30, 124.64, 127.55, 129.45, 129.53, 139.42, 142.91, 154.64, 158.67, 161.06

### Example 39: Synthesis of 6-fluoro-1-(4-methylbenzyl)-2-(5-methyl-2-furyl) benzimidazole (Example 39a) and 5-fluoro-1-(4-methylbenzyl)-2-(5-methyl-2-furyl)benzimidazole (Example 39b)

5-Fluoro-1-(4-methylbenzyl)-2-(5-methyl-2-furyl)benzimidazole (Example 39a) and 6-fluoro-1-(4-methylbenzyl)-2-(5-methyl-2-furyl)benzimidazole (Example 39b) was prepared as in Example 25 at a yield of Example 39a (0.19 g, 24.0%) and Example 39b (0.15 g, 19.0%).

### Example 39a

¹H-NMR (CDCl₃-d₁, 200 MHz) δ(ppm):

2.28 (3H, s, 4″-CH₃), 2.36 (3H, s, 5′-CH₃), 5.55 (2H, s, —CH₂), 6.10 (1H, d, J=3.2, H-4′), 6.86 (1H, d, J=3.4, H-3′), 6.92˜7.23 (6H, m, H-4, 5, 2″, 3″, 5″, 6″), 7.44 (1H, dd, J=2.4, 9.4, H-7)

¹³C-NMR (CDCl₃-d₁, 50 MHz) δ(ppm):

13.82, 21.04, 48.23, 108.33, 110.59, 114.34, 119.36, 123.29, 126.13, 128.32, 129.64, 132.92, 134.47, 137.64, 142.83, 143.95, 145.86, 154.95

### Example 39b

¹H-NMR (CDCl₃-d₁, 200 MHz) δ(ppm):

2.28 (3H, s, 4″-CH₃), 2.35 (3H, s, 5′-CH₃), 5.52 (2H, s, —CH₂), 6.09 (1H, d, J=2.7, H-4′), 6.85 (1H, d, J=3.3, H-3′), 6.88˜7.23 (6H, m, H-6, 7, 2″, 3″, 5″, 6″), 7.69 (1H, dd, J=4.8, 8.7, H-4)

¹³C-NMR (CDCl₃-d₁, 50 MHz) δ(ppm):

13.80, 21.04, 48.28, 96.39, 96.94, 108.20, 110.85, 111.34, 113.69, 120.30, 120.50, 126.19, 129.66, 132.85, 135.87, 137.65, 139.46, 142.96, 154.64

### Example 40: Synthesis of 5-fluoro-1-(3-methylbenzyl)-2-(5-methyl-2-furyl)benzimidazole (Example 40a) and 6-Fluoro-1-(3-methylbenzyl)-2-(5-methyl-2-furyl)benzimidazole (Example 40b)

5-Fluoro-1-(3-methylbenzyl)-2-(5-methyl-2-furyl)benzimidazole (Example 40a) and 6-fluoro-1-(3-methylbenzyl)-2-(5-methyl-2-furyl)benzimidazole (Example 40b) was prepared as in Example 25 at a yield of Example 40a (0.20 g, 26.0%) and Example 40b (0.11 g, 14.0%)

### Example 40a

¹H-NMR (CDCl₃-d₁, 200 MHz) δ(ppm):

2.25 (3H, s, 3″-CH₃), 2.36 (3H, s, 5′-CH₃), 5.56 (2H, s, —CH₂), 6.09 (1H, d, J=3.14 Hz, H-4′), 6.83˜7.23 (7H, m, H-6, 7, 3′, 2″, 4″, 5″, 6″), 7.41 (1H, dd, J=2.28, 9.36 Hz, H-4)

¹³C-NMR (CDCl₃-d₁, 50 MHz) δ(ppm):

13.82, 21.42, 29.70, 48.42, 105.17, 105.65, 108.28, 110.03, 110.23, 110.92, 111.44, 114.04, 123.24, 126.76, 128.60, 128.86, 132.40, 136.03, 138.78, 142.91, 143.48, 143.73, 146.16, 154.85

### Example 40b

¹H-NMR (CDCl₃-d₁, 50 MHz) δ(ppm):

2.26 (3H, s, 3″-CH₃), 2.36 (3H, s, 5′-CH₃), 5.53 (2H, s, —CH₂), 6.09˜6.11 (1H, m, H-4′), 6.88˜7.23 (7H, m, H-5, 7, 3′, 2″, 4″, 5″, 6″), 7.41 (1H, dd, J=4.84, 8.76 Hz, H-4)

¹³C-NMR (CDCl₃-d₁, 50 MHz) δ(ppm):

13.78, 21.40, 29.69, 48.45, 96.38, 96.93, 108.20, 110.85, 111.35, 113.65, 120.32, 120.52, 123.28, 126.80, 128.64, 128.88, 135.86, 136.20, 138.79, 139.51, 142.94, 145.61, 154.63, 157.44

### Example 41: Synthesis of 5-fluoro-1-(2-methylbenzyl)-2-(5-methyl-2-furyl)benzimidazole (Example 41a) and 6-fluoro-1-(2-methylbenzyl)-2-(5-methyl-2-furyl)benzimidazole (Example 41b)

5-Fluoro-1-(2-methylbenzyl)-2-(5-methyl-2-furyl)benzimidazole (Example 41a) and 6-fluoro-1-(2-methylbenzyl)-2-(5-methyl-2-furyl)benzimidazole (Example 41b) was prepared as in Example 25 at a yield of Example 41a (0.15 g, 19.0%) and Example 41b (0.11 g, 14.0%)

### Example 41a

¹H-NMR (CDCl₃-d₁, 200 MHz) δ(ppm):

2.30 (3H, s, 2″-CH₃), 2.40 (3H, s, 5′-CH₃), 5.48 (2H, s, —CH₂), 6.04 (1H, d, J=2.46 Hz, H-4′), 6.45 (1H, d, J=7.52 Hz, H-3″), 6.65 (1H, d, J=3.06 Hz, H-3′), 6.85˜7.23 (5H, m, H-6, 7, 4″, 5″, 6″), 7.43˜7.49 (1H, m, H-4)

¹³C-NMR (CDCl₃-d₁, 50 MHz) δ(ppm):

13.73, 19.15, 46.46, 105.23, 105.71, 108.26, 109.92, 110.12, 110.95, 111.47, 113.85, 124.87, 126.65, 127.59, 130.47, 132.43, 133.89, 134.45, 143.71, 146.35, 154.91

### Example 41b

¹H-NMR (CDCl₃-d₁, 200 MHz) δ(ppm):

2.31 (3H, s, 2″-CH₃), 2.41 (3H, s, 5′-CH₃), 5.49 (2H, s, —CH₂), 6.04 (1H, dd, J=0.9, 3.32 Hz, H-4′), 6.48 (1H, d, J=7.7 Hz, H-3″), 6.63 (1H, d, J=3.34 Hz, H-3′), 6.80 (1H, dd, J=2.36, 8.66 Hz, H-6″), 6.95˜7.25 (4H, m, H-5, 7, 4″, 5″), 7.69 (1H, dd, J=4.82, 8.82, H-4)

¹³C-NMR (CDCl₃-d₁, 50 MHz) δ(ppm):

13.69, 19.12, 46.49, 96.24, 96.79, 108.16, 110.91, 111.41, 113.46, 120.40, 120.60, 124.90, 126.66, 127.62, 130.49, 133.71, 134.46, 135.99, 136.25, 139.48, 142.77, 145.73, 154.68, 157.47, 162.24

### Example 42: Synthesis of 1-benzyl-6-chloro-2-(5-methyl-2-furyl)benzimidazole (Example 42a) and 1-benzyl-5-chloro-2-(5-methyl-2-furyl)benzimidazole (Example 42b)

1-Benzyl-6-chloro-2-(5-methyl-2-furyl)benzimidazole (Example 42a) and 1-benzyl-5-chloro-2-(5-methyl-2-furyl)benzimidazole (Example 42b) was prepared as in Example 25 at a yield of Example 42a (0.27 g, 35.0%) and Example 42b (0.11 g, 14.0%)

### Example 42a

¹H-NMR (CDCl₃-d₁, 200 MHz) δ(ppm): (Chart 46-1)

2.35 (3H, s, 5′-CH₃), 5.59 (2H, s, —CH₂), 6.10 (1H, d, J=2.5, H-4′), 6.87 (1H, d, J=3.2, H-3′), 7.06˜7.28 (6H, m, H-4, 5, 2″, 3″, 5″, 6″), 7.75 (1H, s, H-7)

¹³C-NMR (CDCl₃-d₁, 50 MHz) δ(ppm): (Chart 46-2)

13.82, 48.43, 108.35, 110.53, 114.38, 119.41, 123.36, 126.15, 127.87, 128.40, 128.99, 134.46, 135.98, 142.78, 143.94, 145.87, 155.00

### Example 42b

¹H-NMR (CDCl₃-d₁, 200 MHz) δ(ppm): (Chart 47-1)

2.34 (3H, s, 5′-CH₃), 5.57 (2H, s, —CH₂), 6.10 (1H, d, J=2.3, H-4′), 6.86 (1H, d, J=3.1, H-3′), 7.07˜7.29 (6H, m, H-6, 7, 2″, 3″, 5″, 6″), 7.69 (1H, dd, J=2.1, 9.1, H-4)

¹³C-NMR (CDCl₃-d₁, 50 MHz) δ(ppm): (Chart 47-2)

113.80, 48.40, 108.33, 109.84, 114.21, 120.52, 123.53, 126.13, 127.90, 128.65, 129.03, 135.88, 136.48, 141.74, 142.78, 145.57, 154.92

### Example 43: Synthesis of 6-chloro-1-(4-chlorobenzyl)-2-(5-methyl-2-furyl) benzimidazole (Example 43a) and 5-chloro-1-(4-chlorobenzyl)-2-(5-methyl-2-furyl)benzimidazole (Example 43b)

6-Chloro-1-(4-chlorobenzyl)-2-(5-methyl-2-furyl) benzimidazole (Example 43a) and 5-chloro-1-(4-chlorobenzyl)-2-(5-methyl-2-furyl)benzimidazole (Example 43b) was prepared as in Example 25 at a yield of Example 43a (0.20 g, 23.0%) and Example 43b (0.13 g, 15.0%)

### Example 43a

¹H-NMR (CDCl₃-d₁, 200 MHz) δ(ppm):

2.34 (3H, s, 5′-CH₃), 5.57 (2H, s, —CH₂), 6.12 (1H, d, J=3.1, H-4′), 6.90 (1H, d, J=3.2, H-3′), 7.01 (2H, d, J=8.2, H-2″, 6″), 7.12˜7.15 (2H, m, H-4, 5), 7.25 (2H, d, J=8.3, H-3″, 5″), 7.74 (1H, s, H-7)

¹³C-NMR (CDCl₃-d₁, 50 MHz) δ(ppm):

13.82, 47.88, 64.40, 108.49, 110.33, 114.69, 119.42, 123.52, 127.56, 128.23, 128.61, 129.18, 133.79, 134.21, 134.55, 139.45, 142.69, 143.83, 155.05

### Example 43b

¹H-NMR (CDCl₃-d₁, 200 MHz) δ(ppm):

2.33 (3H, s, 5′-CH₃), 5.54 (2H, s, —CH₂), 6.11 (1H, d, J=2.8, H-4′), 6.89 (1H, d, J=3.3, H-3′), 7.02 (2H, d, J=8.3, H-2″, 6″), 7.19˜7.28 (4H, m, H-6, 7, 3″, 5″), 7.67 (1H, d, J=8.8, H-4)

¹³C-NMR (CDCl₃-d₁, 50 MHz) δ(ppm): (Chart 49-2)

13.82, 47.88, 64.40, 108.49, 110.33, 114.69, 119.42, 123.52, 127.56, 128.23, 128.61, 129.18, 133.79, 134.21, 134.55, 139.45, 142.69, 143.83, 155.05

13.80, 47.83, 108.43, 109.64, 114.35, 120.61, 123.69, 127.53, 128.80, 129.22, 133.81, 134.46, 136.28, 141.75, 142.76, 145.41, 154.95

### Example 44: Synthesis of 5-chloro-1-(3-chlorobenzyl)-2-(5-methyl-2-furyl)benzimidazole (Example 44a) and 6-chloro-1-(3-chlorobenzyl)-2-(5-methyl-2-furyl)benzimidazole (Example 44b)

5-Chloro-1-(3-chlorobenzyl)-2-(5-methyl-2-furyl)benzimidazole (Example 44a) and 6-chloro-1-(3-chlorobenzyl)-2-(5-methyl-2-furyl)benzimidazole (Example 44b) was prepared as in Example 25 at a yield of Example 44a (0.26 g, 30.0%) and Example 44b (0.19 g, 18.0%).

### Example 44a

¹H-NMR (CDCl₃-d₁, 200 MHz) δ(ppm):

2.35 (3H, s, 5′-CH₃), 5.57 (2H, s, —CH₂), 6.11 (1H, d, J=3.24. Hz, H-4′), 6.89˜6.93 (3H, m, H-3′, 2″), 7.23˜7.10 (4H, m, H-6, 7, 4″, 5″, 6″), 7.74 (1H, d, J=1.62 Hz, H-4)

¹³C-NMR (CDCl₃-d₁, 50 MHz) δ(ppm):

13.83, 47.92, 108.47, 110.27, 114.51, 119.54, 123.53, 124.27, 126.44, 128.16, 128.58, 130.32, 134.30, 134.96, 138.16, 142.74, 143.99, 145.74, 155.06

### Example 44b

¹H-NMR (DMSO-d₆, 200 MHz) δ(ppm):

2.34 (3H, s, 5′-CH₃), 5.79 (2H, s, —CH₂), 6.32 (1H, s, H-4′), 6.97˜7.32 (6H, m, H-7,3′, 2″, 4″, 5″, 6″), 7.66 (1H, d, H-5), 7.82 (1H, s, H-4)

¹³C-NMR (DMSO-d₆, 50 MHz) δ(ppm):

13.79, 47.92, 108.49, 109.61, 114.56, 120.56, 123.80, 124.25, 126.42, 128.21, 128.90, 130.35, 134.99, 136.25, 138.02, 141.57, 142.62, 145.36, 155.04

### Example 45: Synthesis of 5-chloro-1-(2-chlorobenzyl)-2-(5-methyl-2-furyl)benzimidazole (Example 45a) and 6-chloro-1-(2-chlorobenzyl)-2-(5-methyl-2-furyl)benzimidazole (Example 45b)

5-Chloro-1-(2-chlorobenzyl)-2-(5-methyl-2-furyl)benzimidazole (Example 45a) and 6-chloro-1-(2-chlorobenzyl)-2-(5-methyl-2-furyl)benzimidazole (Example 45b) was prepared as in Example 25 at a yield of Example 45a (0.25 g, 29.0%) and Example 45b (0.27 g, 31.0%).

### Example 45a

¹H-NMR (CDCl₃-d₁, 200 MHz) δ(ppm):

2.30 (3H, s, 5′-CH₃), 5.65 (2H, s, —CH₂), 6.07 (1H, dd, J=0.88, 3.36 Hz, H-4′), 6.51 (1H, d, H-6″), 6.78 (1H, d, J=3.34 Hz, H-3′), 7.03˜7.23 (4H, m, H-6, 7, 4″, 5″), 7.45 (1H, dd, J=1.08, 6.82 Hz, H-3″), 7.77 (1H, dd, J=0.36, 1.32 Hz, H-4)

¹³C-NMR (CDCl₃-d₁, 50 MHz) δ(ppm):

13.73, 46.15, 108.36, 110.19, 114.23, 119.55, 123.53, 126.61, 127.47, 128.59, 128.99, 129.64, 131.98, 133.58, 134.40, 142.55, 143.56, 145.94, 155.20

### Example 45b

¹H-NMR (CDCl₃-d₁, 200 MHz) δ(ppm):

2.30 (3H, s, 5′-CH₃), 5.64 (2H, s, —CH₂), 6.07 (1H, dd, J=0.92, 3.38. Hz, H-4′), 6.53 (1H, dd, J=1.06, 7.66, H-6″), 6.78 (1H, d, J=3.38 Hz, H-3′), 7.06 (1H, ddd, J=1.24, 7.7, 44, H-5″), 7.17˜7.27 (3H, m, H-5, 7, 4″), 7.46 (1H, dd, J=1.08, 7.94 Hz, H-3″), 7.73 (1H, dd, J=0.4, 8.34 Hz, H-4)

¹³C-NMR (CDCl₃-d₁, 50 MHz) δ(ppm):

13.71, 46.17, 108.34, 109.53, 114.05, 120.67, 123.74, 126.56, 127.51, 128.86, 129.00, 129.66, 131.98, 133.48, 136.43, 141.76, 142.55, 145.64, 155.12

### Example 46: Synthesis of 6-chloro-1-(4-fluorobenzyl)-2-(5-methyl-2-furyl)benzimidazole (Example 46a) and 5chloro-1-(4-fluorobenzyl)-2-(5-methyl-2-furyl)benzimidazole (Example 46b)

5-Chloro-1-(4-fluorobenzyl)-2-(5-methyl-2-furyl)benzimidazole (Example 46a) and 6-chloro-1-(4-fluorobenzyl)-2-(5-methyl-2-furyl)benzimidazole (Example 46b) was prepared as in Example 25 at a yield of Example 46a (0.29 g, 35.0%) and Example 46b (0.16 g, 20.0%).

### Example 46a

¹H-NMR (CDCl₃-d₁, 200 MHz) δ(ppm): (Chart 50-1)

2.35 (3H, s, 5′-CH₃), 5.56 (2H, s, —CH₂), 6.12 (1H, d, J=2.5, H-4′), 6.90 (1H, d, J=3.3, H-3′), 6.96˜7.23 (6H, m, H-4, 5, 2″, 3″, 5″, 6″), 7.73 (1H, s, H-7)

¹³C-NMR (CDCl₃-d₁, 50 MHz) δ(ppm): (Chart 50-2)

13.82, 47.88, 64.40, 108.49, 110.33, 114.69, 119.42, 123.52, 127.56, 128.23, 128.61, 129.18, 133.79, 134.21, 134.55, 139.45, 142.69, 143.83, 155.05

13.80, 47.81, 108.43, 110.37, 114.51, 115.73, 116.16, 119.46, 123.43, 127.83, 127.99, 128.51, 131.81, 134.27, 142.80, 143.95, 145.71, 154.99

### Example 46b

¹H-NMR (CDCl₃-d₁, 200 MHz) δ(ppm): (Chart 51-1)

2.34 (3H, s, 5′-CH₃), 5.54 (2H, s, —CH₂), 6.11 (1H, d, J=2.6, H-4′), 6.89 (1H, d, J=3.3, H-3′), 6.92˜7.23 (6H, m, H-6, 7, 2″, 3″, 5″, 6″), 7.67 (1H, dd, J=3.5, 5.5, H-4)

¹³C-NMR (CDCl₃-d₁, 50 MHz) δ(ppm): (Chart 51-2)

13.79, 47.78, 108.40, 109.69, 114.30, 115.77, 116.20, 120.58, 123.62, 127.82, 127.98, 128.74, 131.71, 136.30, 141.77, 142.81, 145.41, 154.91

### Example 47: Synthesis of 5-chloro-1-(3-fluorobenzyl)-2-(5-methyl-2-furyl)benzimidazole (Example 47a) and 6-chloro-1-(3-fluorobenzyl)-2-(5-methyl-2-furyl)benzimidazole (Example 47b)

5-Chloro-1-(3-fluorobenzyl)-2-(5-methyl-2-furyl)benzimidazole (Example 47a) and 6-chloro-1-(3-fluorobenzyl)-2-(5-methyl-2-furyl)benzimidazole (Example 47b) was prepared as in Example 25 at a yield of Example 47a (0.26 g, 32.0%) and Example 47b (0.19 g, 23.0%).

### Example 47a

¹H-NMR (DMSO-d₆, 400 MHz) δ(ppm):

2.34 (3H, s, 5′-CH₃), 5.80 (2H, s, —CH₂), 6.34 (1H, s, H-4′), 6.87 (1H, d, J=3.77 Hz, H-3′), 6.98 (1H, d, J=4.90 Hz, H-7), 7.08 (2H, m, H-2″, 6″), 7.27˜7.37 (2H, m, H-4″, 5″), 7.67 (1H, d, J=4.30 Hz, H-6), 7.72 (1H, s, H-4)

¹³C-NMR (DMSO-d₆, 100 MHz) δ(ppm):

13.75, 47.51, 109.01, 112.32, 113.64, 113.86, 114.65, 114.86, 114.98, 118.74, 122.61, 123.27, 127.41, 131.23, 131.31, 135.02, 142.83, 143.90, 145.58, 155.03

### Example 47b

¹H-NMR (CDCl₃-d₁, 200 MHz) δ(ppm):

2.34 (3H, s, 5′-CH₃), 5.57 (2H, s, —CH₂), 6.12 (1H, d, J=3.16 Hz, H-4′), 6.77˜7.00 (4H, m, H-7, 3′, 2″, 6″), 7.21 (3H, m, H-6, 4″, 5″), 7.70 (1H, dd, J=1.84, 7.36 Hz, H-4)

¹³C-NMR (CDCl₃-d₁, 50 MHz) δ(ppm):

13.82, 47.94, 108.42, 109.62, 113.06, 113.51, 114.30, 114.76, 115.18, 120.65, 121.73, 123.71, 128.80, 130.63, 130.79, 136.33, 141.78, 142.73, 154.99

### Example 48: Synthesis of 5-chloro-1-(2-fluorobenzyl)-2-(5-methyl-2-furyl)benzimidazole (Example 48a) and 6-chloro-1-(2-fluorobenzyl)-2-(5-methyl-2-furyl)benzimidazole (Example 48b)

5-Chloro-1-(2-fluorobenzyl)-2-(5-methyl-2-furyl)benzimidazole (Example 48a) and 6-chloro-1-(2-fluorobenzyl)-2-(5-methyl-2-furyl)benzimidazole (Example 48b) was prepared as in Example 25 at a yield of Example 48a (0.29 g, 35.0%) and Example 48b (0.27 g, 32.0%).

### Example 48a

¹H-NMR (CDCl₃-d₁, 200 MHz) δ(ppm):

2.33 (3H, s, 5′-CH₃), 5.66 (2H, s, —CH₂), 6.11 (1H, dd, J=0.78, 3.31, H-4′), 6.68 (1H, m, H-5″), 6.89˜7.23 (6H, m, H-6, 7, 3′, 3″, 4″, 6″), 7.74 (1H, s, H-4)

¹³C-NMR (CDCl₃-d₁, 50 MHz) δ(ppm):

13.75, 42.16, 108.40, 110.25, 114.35, 115.26, 115.68, 119.46, 123.31, 124.69, 127.54, 128.50, 129.56, 134.40, 142.78, 143.95, 145.84, 155.07, 157.45, 162.34

### Example 48b

¹H-NMR (CDCl₃-d₁, 200 MHz) δ(ppm):

2.32 (3H, s, 5′-CH₃), 5.64 (2H, s, —CH₂), 6.11 (1H, dd, J=0.68, 3.80, H-4′), 6.69˜6.87 (1H, m, H-5″), 6.89˜7.25 (6H, m, H-5, 7, 3′, 3″, 4″, 6″), 7.70 (1H, d, J=8.43 Hz, H-4)

¹³C-NMR (CDCl₃-d₁, 50 MHz) δ(ppm):

13.74, 42.18, 108.37, 109.58, 114.16, 115.29, 115.71, 120.59, 123.09, 123.37, 123.66, 124.69, 127.44, 128.77, 129.57, 136.43, 141.75, 142.78, 154.99

### Example 49: Synthesis of 5-chloro-1-(4-methoxybenzyl)-2-(5-methyl-2-furyl)benzimidazole (Example 49a) and 6-chloro-1-(4-methoxybenzyl)-2-(5-methyl-2-furyl)benzimidazole (Example 49b)

5-Chloro-1-(4-methoxybenzyl)-2-(5-methyl-2-furyl)benzimidazole (Example 49a) and 6-chloro-1-(4-methoxybenzyl)-2-(5-methyl-2-furyl)benzimidazole (Example 49b) was prepared as in Example 25 at a yield of Example 49a (0.17 g, 13.0%) and Example 49b (0.10 g, 11.0%).

### Example 49a

¹H-NMR (CDCl₃-d₁, 400 MHz) δ(ppm):

2.41 (3H, s, 5′-CH₃), 3.80 (3H, s, 4″-OCH₃), 5.56 (2H, s, —CH₂), 6.15 (1H, d, J=2.80 Hz, H-4′), 6.83 (2H, d, J=8.40, H-3″, 5″), 6.88 (1H, d, J=8.40 Hz, H-6), 6.93 (1H, d, J 3.2, H-3′), 7.05 (2H, d, J=8.40 Hz, H-2″, 6″), 7.28˜7.31 (1H, m, H-7), 7.63 (1H, s, H-4)

¹³C-NMR (CDCl₃-d₁, 100 MHz) δ(ppm):

13.77, 47.90, 55.21, 64.86, 108.31, 110.55, 113.87, 114.30, 119.31, 123.23, 127.48, 127.92, 128.27, 128.54, 134.37, 142.83, 143.92, 145.76, 154.86, 159.16

### Example 49b

¹H-NMR (CDCl₃-d₁, 200 MHz) δ(ppm):

2.41 (3H, s, 5′-CH₃), 3.79 (3H, s, 4″-OCH₃), 5.56 (2H, s, —CH₂), 6.16 (1H, s, H-4′), 6.85 (2H, d, J=8.40, H-3″, 5″), 6.93 (1H, d, J 1.6, H-3′), 7.07 (2H, d, J=8.40 Hz, H-2″, 6″), 7.24˜7.36 (2H, m, H-5, 7), 7.71 (1H, d, J 8.4, H-4)

¹³C-NMR (CDCl₃-d₁, 50 MHz) δ(ppm):

13.76, 47.87, 55.21, 108.27, 109.85, 114.13, 114.35, 120.45, 123.40, 127.46, 127.80, 128.53, 136.38, 141.73, 142.83, 145.46, 154.79, 159.19

### Example 50: Synthesis of 5-chloro-1-(3-methoxybenzyl)-2-(5-methyl-2-furyl)benzimidazole (Example 50a) and 6-chloro-1-(3-methoxybenzyl)-2-(5-methyl-2-furyl)benzimidazole (Example 50b)

5-Chloro-1-(3-methoxybenzyl)-2-(5-methyl-2-furyl)benzimidazole (Example 50a) and 6-chloro-1-(3-methoxybenzyl)-2-(5-methyl-2-furyl)benzimidazole (Example 50b) was prepared as in Example 25 at a yield of Example 50a (0.28 g, 32.0%) and Example 50b (0.19 g, 21.0%).

### Example 50a

¹H-NMR (CDCl₃-d₁, 200 MHz) δ(ppm):

2.35 (3H, s, 5′-CH₃), 3.79 (3H, s, 3″-OCH₃), 5.55 (2H, s, —CH₂), 6.10 (1H, d, J=2.30 Hz, H-4′), 6.61˜6.86 (4H, m, H-3′, 2″, 4″, 6″), 7.14˜7.23 (3H, m, H-6, 7, 5″), 7.74 (1H, s, H-4)

¹³C-NMR (CDCl₃-d₁, 50 MHz) δ(ppm):

13.84, 48.34, 55.19, 108.36, 110.53, 112.15, 112.85, 114.36, 118.36, 119.42, 123.36, 128.38, 130.11, 134.48, 137.59, 142.74, 143.94, 145.88, 155.01, 160.10

### Example 50a

¹H-NMR (CDCl₃-d₁, 200 MHz) δ(ppm):

2.35 (3H, s, 5′-CH₃), 3.70 (3H, s, 3″-OCH₃), 5.54 (2H, s, —CH₂), 6.10 (1H, d, J=2.74 Hz, H-4′), 6.62˜6.86 (4H, m, H-3′, 2″, 4″, 6″), 7.17˜7.23 (3H, m, H-5, 7, 5″), 7.74 (1H, d, J=9.12 Hz, H-4)

¹³C-NMR (CDCl₃-d₁, 50 MHz) δ(ppm):

13.83, 48.30, 55.20, 108.32, 109.83, 112.14, 112.86, 114.14, 118.34, 120.56, 123.53, 128.64, 130.15, 136.49, 137.48, 142.75, 154.93, 160.13

### Example 51: Synthesis of 6-chloro-1-(4-methylbenzyl)-2-(5-methyl-2-furyl)benzimidazole (Example 51a) and 5-chloro-1-(4-methylbenzyl)-2-(5-methyl-2-furyl)benzimidazole (Example 51b)

6-Chloro-1-(4-methylbenzyl)-2-(5-methyl-2-furyl)benzimidazole (Example 51a) and 5-chloro-1-(4-methylbenzyl)-2-(5-methyl-2-furyl)benzimidazole (Example 5 b) was prepared as in Example 25 at a yield of Example 51a (0.39 g, 48.0%) and Example 51b (0.23 g, 28.0%).

### Example 51a

¹H-NMR (CDCl₃-d₁, 200 MHz) δ(ppm): (Chart 52-1)

2.27 (3H, s, 4″-CH₃), 2.36 (3H, s, 5′-CH₃), 5.54 (2H, s, —CH₂), 6.10 (1H, d, J=3.2, H-4′), 6.86 (1H, d, J=3.3, H-3′), 6.96 (2H, d, J=8.0, H-3″, 5″), 7.07 (2H, d, J=8.0, H-2″, 6″), 7.13 (2H, s, H-4, 5), 7.74 (1H, s, H-7)

¹³C-NMR (CDCl₃-d₁, 50 MHz) δ(ppm): (Chart 52-2)

13.82, 21.04, 48.23, 108.33, 110.59, 114.34, 119.36, 123.29, 126.13, 128.32, 129.64, 132.92, 134.47, 137.64, 142.83, 143.95, 145.86, 154.95

### Example 51b

¹H-NMR (CDCl₃-d₁, 200 MHz) δ(ppm): (Chart 53-1)

2.28 (3H, s, 4″-CH₃), 2.35 (3H, s, 5′-CH₃), 5.52 (2H, s, —CH₂), 6.10 (1H, d, J=3.1, H-4′), 6.85 (1H, d, J=3.1, H-3′), 6.97 (2H, d, J=7.8, H-3″, 5″), 7.09 (2H, d, J=7.8, H-2″, 6″), 7.20 (2H, d, J=7.8, H-6, 7), 7.67 (1H, d, J=9.0, H-4)

¹³C-NMR (CDCl₃-d₁, 50 MHz) δ(ppm): (Chart 53-2)

13.81, 21.05, 48.19, 108.30, 109.90, 114.16, 120.48, 123.46, 126.11, 128.59, 129.68, 132.81, 136.48, 137.66, 141.76, 142.82, 145.57, 154.88

### Example 52: Synthesis of 5-chloro-1-(3-methylbenzyl)-2-(5-methyl-2-furyl)benzimidazole (Example 52a) and 6-chloro-1-(3-methylbenzyl)-2-(5-methyl-2-furyl)benzimidazole (Example 52b)

5-Chloro-1-(3-methylbenzyl)-2-(5-methyl-2-furyl)benzimidazole (Example 52a) and 6-chloro-1-(3-methylbenzyl)-2-(5-methyl-2-furyl)benzimidazole (Example 52b) was prepared as in Example 25 at a yield of Example 52a (0.22 g, 27.0%) and Example 52b (0.21 g, 25.0%).

### Example 52a

¹H-NMR (CDCl₃-d₁, 200 MHz) δ(ppm):

2.25 (3H, s, 3″-CH₃), 2.36 (3H, s, 5′-CH₃), 5.56 (2H, s, —CH₂), 6.09˜6.12 (1H, m, H-4′), 6.84˜7.23 (7H, m, H-6, 7, 3′, 2″, 4″, 5″, 6″), 7.75 (1H, d, J=0.86 Hz, H-4)

¹³C-NMR (CDCl₃-d₁, 50 MHz) δ(ppm):

13.83, 21.44, 48.35, 108.32, 109.88, 114.10, 120.52, 123.18, 123.49, 126.69, 128.67, 128.92, 135.78, 136.54, 138.83, 141.78, 142.75, 145.65, 154.92

### Example 52b

¹H-NMR (CDCl₃-d₁, 200 MHz) δ(ppm):

2.25 (3H, s, 3″-CH₃), 2.35 (3H, s, 5′-CH₃), 5.52 (2H, s, —CH₂), 6.08˜6.10 (1H, m, H-4′), 6.82˜7.22 (7H, m, H-5, 7, 3′, 2″, 4″, 5″, 6″), 7.66 (1H, d, J=8.98 Hz, H-4)

¹³C-NMR (CDCl₃-d₁, 50 MHz) δ(ppm):

13.83, 21.44, 48.35, 108.32, 109.88, 114.10, 120.52, 123.18, 123.49, 126.69, 128.67, 128.92, 135.78, 136.54, 138.83, 141.78, 142.75, 145.65, 154.92

### Example 53: Synthesis of 5-chloro-1-(2-methylbenzyl)-2-(5-methyl-2-furyl)benzimidazole (Example 53a) and 6-chloro-1-(2-methylbenzyl)-2-(5-methyl-2-furyl)benzimidazole (Example 53b)

5-Chloro-1-(2-methylbenzyl)-2-(5-methyl-2-furyl)benzimidazole (Example 53a) and 6-chloro-1-(2-methylbenzyl)-2-(5-methyl-2-furyl)benzimidazole (Example 53b) was prepared as in Example 25 at a yield of Example 53a (0.11 g, 13.0%) and Example 53b (0.15 g, 18.0%).

### Example 53a

¹H-NMR (CDCl₃-d₁, 200 MHz) δ(ppm):

2.36 (3H, s, 2″-CH₃), 2.45 (3H, s, 5′-CH₃), 5.54 (2H, s, —CH₂), 6.11 (1H, s, H-4′), 6.52 (1H, d, J=7.20 Hz, H-3″), 6.75 (1H, s, H-3″), 7.06˜7.26 (5H, m, H-5, 7, 4″, 5″, 6″), 7.82 (1H, s, H-4)

¹³C-NMR (CDCl₃-d₁, 50 MHz) δ(ppm):

13.76, 19.16, 29.70, 46.49, 108.31, 110.46, 114.13, 119.48, 123.38, 124.85, 126.68, 127.63, 128.41, 130.48, 133.76, 134.43, 134.55, 142.59, 143.93, 146.11, 155.05

### Example 53b

¹H-NMR (CDCl₃-d₁, 200 MHz) δ(ppm):

2.30 (3H, s, 2″-CH₃), 2.42 (3H, s, 5′-CH₃), 5.51 (2H, s, —CH₂), 6.06 (1H, d, J=2.42 Hz, H-4′), 6.46 (1H, d, J=7.70 Hz, H-3″), 6.73 (1H, d, J=7.70 Hz, H-3″), 6.97˜7.26 (5H, m, H-5, 7, 4″, 5″, 6″), 7.70 (1H, d, J=8.58 Hz, H-4)

¹³C-NMR (CDCl₃-d₁, 50 MHz) δ(ppm):

13.72, 19.14, 46.51, 108.37, 109.81, 114.36, 120.48, 123.73, 124.77, 126.74, 127.69, 128.83, 130.52, 133.58, 134.40, 136.46, 142.39, 145.65, 155.15

### Example 54: Synthesis of 1-benzyl-5,6-dichloro-2-(5-methyl-2-furyl)benzimidazole

1-Benzyl-5,6-dichloro-2-(5-methyl-2-furyl)benzimidazole (Example 54a) was prepared from 5,6-dichloro-2-(5-methyl-2-furyl)benzimidazole (Example 54) as in Example 25 at a yield of 42.0% (0.42 g).

¹H-NMR (CDCl₃-d₁, 200 MHz) δ(ppm): (Chart 21-1)

2.35 (3H, s, 5′-CH₃), 5.57 (2H, s, —CH₂), 6.11 (1H, d, J=2.5, H-4′), 6.88 (1H, d, J=3.4H-3′), 7.05 (2H, d, J=1.6, H-2″, 6″), 7.23˜7.32 (4H, m, H-7, 3″, 4″, 5″), 7.84 (1H, s, H-4)

¹³C-NMR (CDCl₃-d₁, 50 MHz) δ(ppm): (Chart 21-2)

13.83, 48.58, 108.48, 111.09, 114.83, 120.76, 126.09, 126.84, 126.91, 128.05, 129.11, 135.15, 135.55, 142.54, 146.55, 155.30

### Example 55: Synthesis of 5,6-dichloro-1-(4-nitrobenzyl)-2-(5-methyl-2-furyl)benzimidazole

5,6-Dichloro-1-(4-nitrobenzyl)-2-(5-methyl-2-furyl)benzimidazole was prepared as in Example 25 at a yield of 59.0% (0.57 g).

¹H-NMR (CDCl₃-d₁, 200 MHz) δ(ppm): (Chart 22-1)

2.29 (3H, s, 5′-CH₃), 5.68 (2H, s, —CH₂), 6.13 (1H, d, J=2.3, H-4′), 6.96 (1H, d, J=3.4, H-3′), 7.21˜7.27 (3H, m, H-7, 2″, 6″), 7.82 (1H, s, H-4), 8.16 (2H, d, J=8.6, H-3″, 5″)

¹³C-NMR (CDCl₃-d₁, 50 MHz) δ(ppm): (Chart 22-2)

13.81, 48.08, 108.76, 110.58, 115.32, 120.94, 124.36, 126.93, 127.29, 127.41, 134.74, 142.38, 142.50, 143.02, 146.22, 147.71, 155.42

### Example 56: Synthesis of 5,6-dichloro-1-(3-nitrobenzyl)-2-(5-methyl-2-furyl)benzimidazole

5,6-Dichloro-1-(3-nitrobenzyl)-2-(5-methyl-2-furyl)benzimidazole was prepared as in Example 25 at a yield of 59.0% (0.57 g).

¹H-NMR (CDCl₃-d₁, 200 MHz) δ(ppm): (Chart 23-1)

2.33 (3H, s, 5′-CH₃), 5.68 (2H, s, —CH₂), 6.14 (1H, d, J=3.3, H-4′), 7.00 (1H, d, J=3.4, H-3′), 7.23˜7.34 (2H, m, H-7, 6″), 7.47 (1H, dd, J=7.8, 7.8, H-5″), 7.83 (1H, s, H-4), 8.13 (2H, m, H-2″, 4″)

¹³C-NMR (CDCl₃-d₁, 50 MHz) δ(ppm): (Chart 23-2)

13.83, 47.96, 108.78, 110.56, 115.34, 120.97, 121.55, 123.19, 127.25, 127.37, 130.29, 132.05, 134.79, 138.06, 142.48, 142.57, 148.61, 155.42

### Example 57: Synthesis of 5,6-dichloro-1-(2-nitrobenzyl)-2-(5-methyl-2-furyl)benzimidazole

5,6-Dichloro-1-(2-nitrobenzyl)-2-(5-methyl-2-furyl)benzimidazole was prepared as in Example 25 at a yield of 48.0% (0.45 g).

¹H-NMR (CDCl₃-d₁, 200 MHz) δ(ppm): (Chart 24-1)

2.20 (3H, s, 5′-CH₃), 5.97 (2H, s, —CH₂), 6.07 (1H, d, J=2.2, H-4′), 6.61 (1H, d, J=4.3, H-6″), 6.88 (1H, d, J=3.0, H-3′), 7.32 (1H, s, H-7), 7.45 (2H, dd, J=4.3, 4.4, H-4″, 5″), 7.47 (1H, s, H-4), 8.22 (1H, dd, J=5.1, 4.1, H-3″)

¹³C-NMR (CDCl₃-d₁, 50 MHz) δ(ppm): (Chart 24-2)

13.60, 46.43, 108.60, 110.57, 115.10, 120.98, 125.54, 127.03, 127.35, 127.42, 128.83, 132.29, 134.57, 135.02, 142.28, 142.50, 146.46, 147.27, 155.58

### Example 58: Synthesis of 5,6-Dichloro-1-(4-chlorobenzyl)-2-(5-methyl-2-furyl)benzimidazole

5,6-Dichloro-1-(4-chlorobenzyl)-2-(5-methyl-2-furyl)benzimidazole was prepared as in Example 25 at a yield of 427.0% (0.44 g).

¹H-NMR (CDCl₃-d₁, 200 MHz) δ(ppm): (Chart 25-1)

2.34 (3H, s, 5′-CH₃), 5.53 (2H, s, —CH₂), 6.12 (1H, d, J=1.3, H-4′), 6.91 (1H, d, J=3.0, H-3′), 6.98˜7.02 (2H, m, H-2″, 6″), 7.24˜7.28 (3H, m, H-7, 3″, 5″), 7.82 (1H, s, H-4)

¹³C-NMR (CDCl₃-d₁, 50 MHz) δ(ppm): (Chart 25-2)

13.82, 47.99, 108.59, 110.88, 115.01, 120.80, 127.08, 127.48, 129.29, 133.96, 134.13, 134.93, 142.50, 146.36, 155.33

### Example 59: Synthesis of 5,6-dichloro-1-(3-chlorobenzyl)-2-(5-methyl-2-furyl)benzimidazole

5,6-Dichloro-1-(3-chlorobenzyl)-2-(5-methyl-2-furyl)benzimidazole was prepared as in Example 25 at a yield of 74.0% (0.69 g).

¹H-NMR (DMSO-d₆, 200 MHz) δ(ppm): (Chart 26-1)

2.29 (3H, s, 5′-CH₃), 5.75 (2H, s, —CH₂), 6.28 (1H, d, J=2.7, H-4′), 6.91 (1H, d, J=3.4, H-3′), 7.03 (1H, d, J=3.2, H-2″), 7.19˜7.28 (3H, m, H-4″, 5″, 6″), 7.87 (1H, s, H-7), 8.03 (1H, s, H-4)

¹³C-NMR (DMSO-d₆, 50 MHz) δ(ppm): (Chart 26-2)

13.79, 109.18, 112.64, 115.53, 120.44, 125.26, 125.65, 126.87, 127.96, 131.15, 133.74, 135.93, 139.87, 142.53, 146.38, 155.38

### Example 60: Synthesis of 5,6-dichloro-1-(2-chlorobenzyl)-2-(5-methyl-2-furyl)benzimidazole

5,6-Dichloro-1-(2-chlorobenzyl)-2-(5-methyl-2-furyl)benzimidazole was prepared as in Example 25 at a yield of 46.0% (0.45 g).

¹H-NMR (DMSO-d₆, 200 MHz) δ(ppm): (Chart 27-1)

2.29 (3H, s, 5′-CH₃), 5.62 (2H, s, —CH₂), 6.09 (1H, d, J=2.7, H-4′), 6.49 (1H, d, J=7.5, H-6″), 6.81 (1H, d, J=2.7, H-3′), 7.06 (1H, dd, J=7.4, 7.4, H-5″), 7.20 (1H, dd, J=4.7, 4.7, H-4″), 7.28 (1H, s, H-7), 7.44 (1H, d, J=7.8, H-3″), 7.85 (1H, s, H-4)

¹³C-NMR (DMSO-d₆, 50 MHz) δ(ppm): (Chart 27-2)

13.77, 55.93, 60.94, 103.89, 105.98, 107.35, 107.42, 115.09, 118.54, 125.41, 125.84, 127.69, 128.92, 129.27, 140.06, 143.98, 144.71, 146.71, 152.14, 153.04

13.70, 46.33, 108.50, 110.78, 114.78, 120.83, 126.46, 127.13, 127.54, 129.14, 129.75, 132.02, 133.19, 135.07, 142.24, 142.47, 146.56, 155.51

### Example 61: Synthesis of 5,6-dichloro-1-(4-methoxybenzyl)-2-(5-methyl-2-furyl)benzimidazole

5,6-Dichloro-1-(4-methoxybenzyl)-2-(5-methyl-2-furyl)benzimidazole was prepared as in Example 25 at a yield of 58.0% (0.36 g).

¹H-NMR (CDCl₃-d₁, 200 MHz) δ(ppm): (Chart 28-1)

2.37 (3H, s, 5′-CH₃), 3.74 (3H, s, 4″-OCH₃), 5.50 (2H, s, —CH₂), 6.12 (1H, d, J=2.6, H-4′), 6.81 (2H, dd, J=8.7, H-3″, 5″), 6.90 (1H, d, J=3.3, H-3′), 7.01 (2H, dd, J=8.6, H-2″, 6″), 7.32 (1H, s, H-7), 7.82 (1H, s, H-4)

¹³C-NMR (CDCl₃-d₁, 50 MHz) δ(ppm): (Chart 28-2)

13.85, 48.11, 55.28, 108.49, 111.17, 114.46, 114.84, 120.69, 126.76, 127.49, 135.08, 142.55, 146.46, 155.25, 159.33

### Example 62: Synthesis of 5,6-Dichloro-1-(3-methoxybenzyl)-2-(5-methyl-2-furyl)benzimidazole

5,6-Dichloro-1-(3-methoxybenzyl)-2-(5-methyl-2-furyl)benzimidazole was prepared as in Example 25 at a yield of 53.0% (0.32 g).

¹H-NMR (CDCl₃-d₁, 200 MHz) δ(ppm): (Chart 29-1)

2.35 (3H, s, 5′-CH₃), 3.70 (3H, s, 3″-OCH₃), 5.53 (2H, s, —CH₂), 6.11 (1H, d, J=3.3, H-4′), 6.60˜6.66 (2H, m, H-2″, 4″), 6.78 (1H, d, J=8.2, H-6″), 6.88 (1H, d, J=3.3, H-3′), 7.23 (1H, dd, J=3.7, 3.9, H-5″), 7.31 (1H, s, H-7), 7.83 (1H, s, H-4)

¹³C-NMR (CDCl₃-d₁, 50 MHz) δ(ppm): (Chart 29-2)

13.83, 48.48, 55.21, 108.49, 111.10, 112.17, 112.95, 114.86, 118.28, 120.74, 126.92, 130.23, 135.13, 137.14, 142.50, 146.54, 155.32, 160.19

### Example 63: Synthesis of 5,6-dichloro-1-(4-fluorobenzyl)-2-(5-methyl-2-furyl)benzimidazole

5,6-Dichloro-1-(4-fluorobenzyl)-2-(5-methyl-2-furyl)benzimidazole was prepared as in Example 25 at a yield of 48.0% (0.30 g).

¹H-NMR (CDCl₃-d₁, 200 MHz) δ(ppm): (Chart 30-1)

2.35 (3H, s, 5′-CH₃), 5.54 (2H, s, —CH₂), 6.13 (1H, d, J=2.7, H-4′), 6.91 (1H, d, J=3.3, H-3′), 6.98˜7.23 (4H, m, H-2″, 3″, 5″, 6″), 7.30 (1H, s, H-7), 7.82 (1H, s, H-4)

¹³C-NMR (CDCl₃-d₁, 50 MHz) δ(ppm): (Chart 30-2)

13.84, 47.96, 108.57, 110.94, 114.95, 115.87, 116.30, 120.79, 126.93, 127.01, 127.79, 127.95, 131.33, 134.95, 142.51, 146.37, 155.30

### Example 64: Synthesis of 5,6-dichloro-1-(3-fluorobenzyl)-2-(5-methyl-2-furyl)benzimidazole

5,6-Dichloro-1-(3-fluorobenzyl)-2-(5-methyl-2-furyl)benzimidazole was prepared as in Example 25 at a yield of 52.0% (0.32 g).

¹H-NMR (CDCl₃-d₁, 200 MHz) δ(ppm): (Chart 31-1)

2.34 (3H, s, 5′-CH₃), 5.56 (2H, s, —CH₂), 6.12 (1H, d, J=2.7, H-4′), 6.75 (4H, m, H-3′, 2″, 5″, 6″), 7.22˜7.32 (2H, m, H-7, 4″), 7.83 (1H, s, H-4),

¹³C-NMR (CDCl₃-d₁, 50 MHz) δ(ppm): (Chart 31-2)

13.82, 48.10, 108.58, 110.87, 113.03, 113.48, 114.98, 115.31, 120.83, 121.68, 127.09, 130.70, 134.98, 142.43, 142.53, 146.40, 155.36

### Example 65: Synthesis of 5,6-dichloro-1-(2-fluorobenzyl)-2-(5-methyl-2-furyl) benzimidazole

5,6-Dichloro-1-(2-fluorobenzyl)-2-(5-methyl-2-furyl) benzimidazole was prepared as in Example 25 at a yield of 66.0% (0.40 g).

¹H-NMR (CDCl₃-d₁, 200 MHz) δ(ppm): (Chart 32-1)

2.33 (3H, s, 5′-CH₃), 5.63 (2H, s, —CH₂), 6.12 (1H, d, J=2.5, H-4′), 6.69˜7.27 (5H, m, H-3′, 3″, 4″, 5″, 6″), 7.35 (1H, s, H-7), 7.83 (1H, s, H-4)

¹³C-NMR (CDCl₃-d₁, 50 MHz) δ(ppm): (Chart 32-2)

13.74, 42.43, 108.53, 110.85, 114.89, 115.39, 115.80, 120.78, 122.79, 123.07, 124.80, 127.04, 127.47, 129.67, 135.07, 142.49, 146.47, 155.37

### Example 66: Synthesis of 5,6-dichloro-1-(4-methylbenzyl)-2-(5-methyl-2-furyl)benzimidazole

5,6-Dichloro-1-(4-methylbenzyl)-2-(5-methyl-2-furyl)benzimidazole was prepared as in Example 25 at a yield of 34.0% (0.20 g).

¹H-NMR (CDCl₃-d₁, 200 MHz) δ(ppm): (Chart 33-1)

2.29 (3H, s, 4″-CH₃), 2.36 (3H, s, 5′-CH₃), 5.52 (2H, s, —CH₂), 6.11 (1H, d, J=3.3, H-4′), 6.88 (1H, d, J=3.3, H-3′), 6.96 (2H, d, J=8.0, H-3″, 5″), 7.10 (2H, d, J=7.9, H-2″, 6″), 7.31 (1H, s, H-7), 7.82 (1H, s, H-4)

¹³C-NMR (CDCl₃-d₁, 50 MHz) δ(ppm): (Chart 33-2)

13.84, 21.06, 48.38, 108.46, 111.15, 114.78, 120.70, 126.07, 126.82, 129.76, 132.48, 135.14, 137.85, 142.54, 146.53, 155.27

### Example 67: Synthesis of 5,6-dichloro-1-(3-methylbenzyl)-2-(5-methyl-2-furyl)benzimidazole

5,6-Dichloro-1-(3-methylbenzyl)-2-(5-methyl-2-furyl)benzimidazole was prepared as in Example 25 at a yield of 66.0% (0.40 g).

¹H-NMR (CDCl₃-d₁, 200 MHz) δ(ppm): (Chart 34-1)

2.26 (3H, s, 3″-CH₃), 2.35 (3H, s, 5′-CH₃), 5.52 (2H, s, —CH₂), 6.11 (1H, d, J=2.6, H-4′), 6.85˜7.23 (1H, m, H-3′, 2″, 4″, 5″, 6″), 7.30 (1H, s, H-7), 7.83 (1H, s, H-4)

¹³C-NMR (CDCl₃-d₁, 50 MHz) δ(ppm): (Chart 34-2)

13.83, 21.42, 48.53, 108.73, 111.13, 114.78, 120.71, 123.14, 126.64, 126.85, 128.81, 128.98, 135.18, 135.46, 138.93, 142.46, 142.53, 146.58, 155.30

### Example 68: Synthesis of 5,6-dichloro-1-(2-methylbenzyl)-2-(5-methyl-2-furyl)benzimidazole

5,6-Dichloro-1-(2-methylbenzyl)-2-(5-methyl-2-furyl)benzimidazole was prepared as in Example 25 at a yield of 41.0% (0.24 g).

¹H-NMR (CDCl₃-d₁, 200 MHz) δ(ppm): (Chart 35-1)

2.26 (3H, s, 2″-CH₃), 2.41 (3H, s, 5′-CH₃), 5.48 (2H, s, —CH₂), 6.07 (1H, d, J=3.2, H-4′), 6.07 (1H, d, J=3.2, H-4′), 6.44 (1H, d, J=7.6, H-3″), 6.70 (1H, d, J=3.1, H-3″), 7.01 (1H, dd, J=7.5, 7.5, H-4″), 7.13˜7.26 (3H, m, H-7, 5″, 6″), 7.83 (1H, s, H-4)

¹³C-NMR (CDCl₃-d₁, 50 MHz) δ(ppm): (Chart 35-2)

13.75, 19.14, 46.66, 108.44, 111.07, 114.61, 120.80, 124.68, 126.76, 126.88, 127.80, 130.60, 133.34, 134.45, 135.23, 142.30, 142.51, 146.77, 155.36

### Example 69: Synthesis of 1-benzyl-6-benzoyl-2-(5-methyl-2-furyl)benzimidazole

1-Benzyl-6-benzoyl-2-(5-methyl-2-furyl)benzimidazole (Example 69a) was prepared from 6-benzoyl-2-(5-methyl-2-furyl)benzimidazole (Example 69) as in Example 25 at a yield of 38.0% (0.36 g).

¹H-NMR (CDCl₃-d₁, 200 MHz) δ(ppm): (Chart 4-1)

2.36 (3H, s, 5′-CH₃), 5.67 (2H, s, —CH₂), 6.13 (1H, d, J=3.3, H-4′), 6.91˜7.82 (14H, m, H-4, 5, 7, 3′, 2″, 3″, 4″, 5″, 6″, 2″′, 3″′, 4″′, 5″′, 6″′)

¹³C-NMR (CDCl₃-d₁, 50 MHz) δ(ppm): (Chart 4-2)

13.85, 48.49, 108.45, 108.60, 109.83, 112.59, 114.66, 115.27, 118.96, 123.25, 125.12, 125.78, 126.22, 126.33, 127.92, 128.15, 129.01, 129.95, 130.03, 131.96, 132.13, 135.92, 138.29, 142.87, 146.57, 155.41, 196.27

### Example 70: Synthesis of 6-benzoyl-1-(4-nitrobenzyl)-2-(5-methyl-2-furyl)benzimidazole

6-Benzoyl-1-(4-nitrobenzyl)-2-(5-methyl-2-furyl)benzimidazole was prepared as in Example 25 at a yield of 22.0% (0.23 g).

¹H-NMR (CDCl₃-d₁, 200 MHz) δ(ppm): (Chart 5-1)

2.31 (3H, s, 5′-CH₃), 5.80 (3H, s, —CH₂), 6.16 (1H, d, J=3.3, H-4′), 7.03˜8.19 (10H, m, H-4, 5, 3′, 2″, 6″, 2″′, 3″′, 4″′, 5″′, 6″′), 8.12˜8.19 (3H, m, H-7, 3″, 5″)

¹³C-NMR (CDCl₃-d₁, 50 MHz) δ(ppm): (Chart 5-2)

13.82, 47.75, 62.45, 109.27, 113.34, 115.89, 119.14, 123.71, 124.42, 125.42, 127.47, 127.99, 128.86, 130.01, 132.01, 132.71, 135.88, 138.23, 142.69, 142.81, 145.32, 146.46, 147.11, 147.31, 155.67, 195.76

### Example 71: Synthesis of 6-benzoyl-1-(3-nitrobenzyl)-2-(5-methyl-2-furyl)benzimidazole

6-Benzoyl-1-(3-nitrobenzyl)-2-(5-methyl-2-furyl)benzimidazole was prepared as in Example 25 at a yield of 14.0% (0.15 g).

¹H-NMR (DMSO-d₆, 200 MHz) δ(ppm): (Chart 6-1)

2.31 (3H, s, 5′-CH₃), 5.98 (1H, s, —CH₂), 6.32 (1H, d, J=3.3, H-4′), 7.14˜8.11 (13H, m, H-4, 5, 7, 3′, 2″, 3″, 5″, 6″, 2″′3″′4″′, 5″′, 6′)

¹³C-NMR (CDCl₃-d₁, 50 MHz) δ(ppm): (Chart 6-2)

13.74, 105.05, 107.49, 107.85, 115.58, 116.02, 123.37, 125.57, 126.02,

126.08, 127.69, 127.96, 129.76, 130.49, 130.66, 130.95, 134.70, 140.72,

143.18, 145.17, 146.28, 152.43, 160.33, 165.29

### Example 72: Synthesis of 6-benzoyl-1-(2-nitrobenzyl)-2-(5-methyl-2-furyl)benzimidazole

Synthesis of 6-Benzoyl-1-(2-nitrobenzyl)-2-(5-methyl-2-furyl)benzimidazole was prepared as in Example 25 at a yield of 26.0% (0.28 g).

¹H-NMR (CDCl₃-d₁, 200 MHz) δ(ppm): (Chart 7-1)

2.20 (3H, s, 5′-CH₃), 6.08 (3H, s, —CH₂, H-4′), 6.64 (1H, d, J=4.9, H-6″), 6.98 (1H, d, J=3.2, H-3′), 7.40˜7.86 (10H, m, H-4, 5, 7, 4″, 5″, 2″′, 3″′, 4″′, 5″′, 6″′), 8.20 (1H, t, J=5.7, 3.2, H-3″)

¹³C-NMR (CDCl₃-d₁, 50 MHz) δ(ppm): (Chart 7-2)

13.60, 46.36, 108.69, 111.63, 115.52, 119.10, 125.47, 126.35, 127.14, 128.24, 128.67, 129.93, 132.14, 132.69, 134.47, 135.74, 138.16, 142.60, 146.48, 147.33, 155.76, 196.26

### Example 73: Synthesis of 6-Benzoyl-1-(4-chlorobenzyl)-2-(5-methyl-2-furyl)benzimidazole

6-Benzoyl-1-(4-chlorobenzyl)-2-(5-methyl-2-furyl)benzimidazole was prepared as in Example 25 at a yield of 29.0% (0.30 g).

¹H-NMR (DMSO-d₆, 200 MHz) δ(ppm): (Chart 8-1)

2.32 (3H, s, 5′-CH₃), 5.82 (2H, s, —CH₂), 6.33 (1H, d, J=2.7, H-4′), 7.06˜7.76 (12H, m, H-4, 5, 3′, 2″, 3″, 5″, 6″, 2″′, 3″′, 4′″, 5″′, 6′″), 7.96 (1H, s, H-7)

¹³C-NMR (DMSO-d₆, 50 MHz) δ(ppm): (Chart 8-2)

13.86, 31.11, 47.48, 109.26, 113.56, 115.87, 119.09, 125.25, 128.78, 128.86, 129.23, 130.00, 131.82, 132.59, 132.71, 135.77, 136.45, 138.23, 142.89, 146.47, 155.59, 195.74, 207.01

### Example 74: Synthesis of 6-benzoyl-1-(3-chlorobenzyl)-2-(5-methyl-2-furyl)benzimidazole

6-Benzoyl-1-(3-chlorobenzyl)-2-(5-methyl-2-furyl)benzimidazole was prepared as in Example 25 at a yield of 21.0% (0.22 g).

¹H-NMR (DMSO-d₆, 200 MHz) δ(ppm): (Chart 9-1)

2.32 (3H, s, 5′-CH₃), 5.82 (2H, s, —CH₂), 6.32 (1H, d, J=4.0, H-4′), 7.04˜7.75 (12H, m, H-4, 5, 3′, 2″, 4″, 5″, 6″, 2″′, 3″′, 4′″, 5″′, 6′″), 7.98 (1H, s, H-7)

¹³C-NMR (DMSO-d₆, 50 MHz) δ(ppm): (Chart 9-2)

13.82, 47.52, 109.18, 109.29, 111.05, 113.56, 115.35, 115.88, 119.13, 121.98, 125.38, 126.97, 128.01, 128.88, 129.99, 131.19, 131.87, 132.24, 132.70, 133.79, 135.78, 138.24, 139.98, 142.86, 146.43, 155.28, 155.58

### Example 75: Synthesis of 6-benzoyl-1-(2-chlorobenzyl)-2-(5-methyl-2-furyl)benzimidazole

6-Benzoyl-1-(2-chlorobenzyl)-2-(5-methyl-2-furyl)benzimidazole was prepared as in Example 25 at a yield of 26.0% (0.27 g).

¹H-NMR (CDCl₃-d₁, 200 MHz) δ(ppm): (Chart 10-1)

2.31 (1H, s, 5′-CH₃), 5.75 (2H, s, —CH₂), 6.11 (1H, d, J=3.2, H-4′), 6.53 (1H, d, J=7.6, H-6″), 6.90 (1H, d, J=3.3, H-3′), 7.00˜7.85 (11H, m, H-4, 5, 7, 3″, 4″, 5″, 2″′, 3″′, 4″′, 5″′, 6″′)

¹³C-NMR (CDCl₃-d₁, 50 MHz) δ(ppm): (Chart 10-2)

13.73, 46.25, 108.64, 112.03, 115.19, 119.03, 126.07, 126.59, 127.45, 128.19, 129.00, 129.68, 129.93, 132.05, 132.40, 133.56, 135.73, 138.26, 142.58, 146.49, 147.46, 155.66, 196.29

### Example 76: Synthesis of 6-benzoyl-1-(4-methoxybenzyl)-2-(5-methyl-2-furyl)benzimidazole

6-Benzoyl-1-(4-methoxybenzyl)-2-(5-methyl-2-furyl)benzimidazole was prepared as in Example 25 at a yield of 20.0% (0.20 g).

¹H-NMR (DMSO-d₆, 400 MHz) δ(ppm): (Chart 11-1)

2.40 (3H, s, 5′-CH₃), 3.70 (3H, s, 4″-OCH₃), 5.77 (2H, s, —CH₂), 6.39 (1H, d, J=1.1, H-4′), 6.88 (2H, d, J=7.6, H-3″, 5″), 7.08 (2H, d, J=8.3, H-2″, 6″), 7.17 (1H, d, J=2.8, H-3′), 7.54 (2H, d, J=7.1, H-3″′, 5″′), 7.65 (1H, s, H-5), 7.67˜7.70 (3H, m, H-2″′, 4″′, 6″′), 7.76 (1H, d, J=8.2, H-4), 7.99 (1H, s, H-7)

¹³C-NMR (DMSO-d₆, 50 MHz) δ(ppm): (Chart 11-2)

13.91, 47.56, 55.51, 109.25, 113.83, 114.63, 115.82, 119.03, 125.09, 128.39, 128.87, 129.12, 130.01, 131.60, 132.69, 135.72, 138.27, 143.08, 146.55, 147.09, 155.47, 159.11, 195.72

### Example 77: Synthesis of 6-benzoyl-1-(3-methoxybenzyl)-2-(5-methyl-2-furyl)benzimidazole

6-Benzoyl-1-(3-methoxybenzyl)-2-(5-methyl-2-furyl)benzimidazole was prepared as in Example 25 at a yield of 8.0% (0.10 g).

¹H-NMR (DMSO-d₆, 200 MHz) δ(ppm): (Chart 12-1)

2.31 (3H, s, 5′-CH₃), 3.62 (3H, s, 4″-OCH₃), 5.78 (1H, s, —CH₂), 6.33 (1H, d, J=3.2, H-4′), 6.58˜7.18 (5H, m, H-3′, 2″, 3″, 5″, 6″), 7.50 (2H, dd, J=7.1, H-3″′, 5″′), 7.58˜7.65 (4H, m, H-5, 2″′, 4″′, 6″′), 7.73 (1H, d, J=8.4, H-4), 7.96 (1H, s, H-7)

¹³C-NMR (DMSO-d₆, 50 MHz) δ(ppm): (Chart 12-2)

13.87, 47.99, 55.43, 109.26, 113.06, 113.73, 115.84, 118.82, 119.07, 125.17, 128.86, 129.98, 130.47, 131.68, 132.67, 135.81, 138.24, 138.86, 142.96, 146.47, 155.51, 159.92

### Example 78: Synthesis of 6-benzoyl-1-(4-fluorobenzyl)-2-(5-methyl-2-furyl)benzimidazole

6-Benzoyl-1-(4-fluorobenzyl)-2-(5-methyl-2-furyl)benzimidazole was prepared as in Example 25 at a yield of 21.0% (0.21 g).

¹H-NMR (CDCl₃-d₁, 200 MHz) δ(ppm): (Chart 13-1)

2.33 (3H, s, 5′-CH₃), 5.80 (2H, s, —CH₂), 6.33 (1H, d, J=3.2, H-4′), 7.10˜7.75 (12H, m, H-4, 5, 3′, 2″, 3″, 5″, 6″, 2″′, 3″′, 4′″, 5″′, 6′″), 7.96 (1H, s, H-7)

¹³C-NMR (CDCl₃-d₁, 50 MHz) δ(ppm): (Chart 13-2)

13.87, 47.42, 109.28, 113.64, 115.88, 116.30, 119.08, 125.22, 128.89, 129.10, 130.00, 131.78, 132.74, 133.57, 135.72, 138.22, 142.91, 146.46, 155.61, 195.78

### Example 79: Synthesis of 6-Benzoyl-1-(3-fluorobenzyl)-2-(5-methyl-2-furyl)benzimidazole

6-Benzoyl-1-(3-fluorobenzyl)-2-(5-methyl-2-furyl)benzimidazole was prepared as in Example 25 at a yield of 52.0% (0.51 g).

¹H-NMR (DMSO-d₆, 200 MHz) δ(ppm): (Chart 14-1)

2.32 (3H, s, 5′-CH₃), 5.84 (2H, s, —CH₂), 6.32 (1H, d, J=3.4, H-4′), 6.85˜7.76 (12H, m, H-4, 5, 3′, 2″, 4″, 5″, 6″, 2″′, 3″′, 4′″, 5″′, 6′″), 7.97 (1H, s, H-7)

¹³C-NMR (DMSO-d₆, 50 MHz) δ(ppm): (Chart 14-2)

13.83, 47.62, 109.27, 113.59, 114.65, 115.07, 115.86, 119.11, 122.73, 125.26, 128.85, 130.00, 131.28, 131.45, 131.83, 132.69, 135.79, 138.25, 140.24, 140.39, 142.90, 146.47, 155.56, 195.73

### Example 80: Synthesis of 6-benzoyl-1-(2-fluorobenzyl)-2-(5-methyl-2-furyl)benzimidazole

6-Benzoyl-1-(2-fluorobenzyl)-2-(5-methyl-2-furyl)benzimidazole was prepared as in Example 25 at a yield of 47.0% (0.46 g).

¹H-NMR (CDCl₃-d₁, 200 MHz) δ(ppm): (Chart 15-1)

2.35 (3H, s, furyl C-5′-CH₃), 3.78 (3H, s, phenyl C-4″-OCH₃), 6.04 (1H, dd, J=0.8, 3.1, furyl C-4′-H), 6.61 (1H, d, J=1.4, furyl C-3′-H), 6.62 (1H, s, pyrazole C-4-H), 6.79˜7.32 (8H, m, phenyl-H)

¹³C-NMR (CDCl₃-d₁, 50 MHz) δ(ppm): (Chart 15-2)

13.77, 55.28, 103.97, 107.41, 113.98, 115.55, 116.01, 122.38, 127.22, 127.39, 130.00, 144.03, 144.73, 146.64, 152.17, 159.68

### Example 81: Synthesis of 6-benzoyl-1-(4-methylbenzyl)-2-(5-methyl-2-furyl)benzimidazole

6-Benzoyl-1-(4-methylbenzyl)-2-(5-methyl-2-furyl)benzimidazole was prepared as in Example 25 at a yield of 26.0% (0.25 g).

¹H-NMR (CDCl₃-d₁, 200 MHz) δ(ppm): (Chart 16-1)

2.28 (3H, s, 5′-CH₃), 2.37 (3H, s, 4″-CH₃), 5.64 (1H, s, —CH₂), 6.14 (1H, d, J=3.1, H-4′), 6.98˜7.77 (12H, m, H-4, 5, 3′, 2″, 3″, 5″, 6″, 2″′, 3″′, 4″′, 5″′, 6″′), 7.83 (1H, s, H-7)

¹³C-NMR (CDCl₃-d₁, 50 MHz) δ(ppm): (Chart 16-2)

13.87, 21.06, 48.29, 108.58, 112.67, 115.21, 118.93, 125.74, 126.33, 128.14, 129.65, 129.96, 131.94, 132.08, 132.86, 135.66, 137.66, 138.34, 142.91, 146.60, 147.36, 155.37, 196.30

### Example 82: Synthesis of 6-benzoyl-1-(3-methylbenzyl)-2-(5-methyl-2-furyl) benzimidazole

6-Benzoyl-1-(3-methylbenzyl)-2-(5-methyl-2-furyl) benzimidazole was prepared as in Example 25 at a yield of 49.0% (0.48 g).

¹H-NMR (CDCl₃-d₁, 200 MHz) δ(ppm): (Chart 17-1)

2.24 (3H, s, 5′-CH₃), 2.36 (3H, s, 3″-CH₃), 5.63 (1H, s, —CH₂), 6.13 (1H, d, J=3.4, H-4′), 6.91˜7.84 (13H, m, H-4, 5, 7, 3′, 2″, 3″, 5″, 6″, 2″′, 3″′, 4″′, 5″′, 6″′)

¹³C-NMR (CDCl₃-d₁, 50 MHz) δ(ppm): (Chart 17-2)

13.85, 21.41, 48.50, 108.60, 112.68, 114.63, 115.23, 118.95, 123.29, 123.39, 125.11, 125.74, 126.81, 126.93, 128.14, 128.70, 128.90, 129.95, 130.03, 132.00, 132.11, 135.83, 138.82, 142.84, 146.56, 155.11, 155.40, 196.31

### Example 83: Synthesis of 6-benzoyl-1-(2-methylbenzyl)-2-(5-methyl-2-furyl)benzimidazole

6-Benzoyl-1-(2-methylbenzyl)-2-(5-methyl-2-furyl)benzimidazole was prepared as in Example 25 at a yield of 25.0% (0.24 g).

¹H-NMR (DMSO-d₆, 200 MHz) δ(ppm): (Chart 18-1)

2.25 (3H, s, 5′-CH₃), 2.34 (3H, s, 2″-CH₃), 5.76 (1H, s, —CH₂), 6.25 (1H, d, J=5.0, H-4′), 6.94˜7.74 (12H, m, H-4, 5, 3′, 2″, 3″, 5″, 6″, 2″′, 3″′, 4″′, 5″′, 6″′), 7.86 (1H, s, H-7)

¹³C-NMR (CDCl₃-d₁, 50 MHz) δ(ppm): (Chart 18-2)

13.75, 19.18, 46.42, 109.47, 113.50, 115.70, 119.01, 124.48, 125.31, 126.70, 127.62, 128.88, 129.95, 130.75, 131.80, 132.65, 135.29, 135.42, 136.05, 138.23, 142.83, 155.46, 195.74

### Example 84: Synthesis of 6-benzoyl-1-(2,5-dichlorobenzyl)-2-(5-methyl-2-furyl)benzimidazole

6-Benzoyl-1-(2,5-dichlorobenzyl)-2-(5-methyl-2-furyl)benzimidazole was prepared as in Example 25 at a yield of 66.0% (0.77 g).

¹H-NMR (CDCl₃-d₁, 200 MHz) δ(ppm): (Chart 19-1)

2.41 (3H, s, furyl C-5-CH₃), 6.19 (1H, d, J=3.3, furyl C-4-H), 7.25 (1H, d, J=11.6, α-H), 7.34 (2H, d, J=8.4, phenyl C-3′,5′-H), 7.36 (1H, s, furyl C-3-H), 7.53 (2H, d, J=8.5, phenyl C-2′,6′-H), 7.75 (1H, d, J=15.8, α-H)

¹³C-NMR (CDCl₃-d₁, 50 MHz) δ(ppm): (Chart 19-2)

14.20, 109.45, 119.76, 121.75, 129.18, 129.58, 133.36, 136.26, 141.79, 152.41, 158.33, 176.97

### Example 85: Synthesis of 6-acetyl-1-(3-chlorobenzyl)-2-(5-methyl-2-furyl)benzimidazole (Example 85a) and 5-acetyl-1-(3-chlorobenzyl)-2-(5-methyl-2-furyl)benzimidazole (Example 85b)

6-Acetyl-1-(3-chlorobenzyl)-2-(5-methyl-2-furyl)benzimidazole (Example 85a) and 5-acetyl-1-(3-chlorobenzyl)-2-(5-methyl-2-furyl)benzimidazole (Example 85b) was prepared as in Example 25 at a yield of Example 85a (0.15 g, 20.0%) and Example 85b (0.10 g, 12.0%)

### Example 85a

¹H-NMR (DMSO-d₆, 200 MHz) δ(ppm):

2.30 (3H, s, 5′-CH₃), 2.57 (3H, s, 6-COCH₃), 5.86 (2H, s, —CH₂), 6.28 (1H, d, J=2.54 Hz, H-4′), 6.94˜6.95 (1H, m, H-5″), 7.06 (1H, d, J=3.20 Hz, H-3′), 7.19 (1H, s, H-2″), 7.25˜7.28 (2H, m, H-4″, 6″), 7.67 (1H, d, J=8.46 Hz, H-4), 7.83 (1H, d, J=8.48 Hz, H-5), 7.67 (1H, s, H-7)

¹³C-NMR (DMSO-d₆, 50 MHz) δ(ppm):

13.79, 27.27, 47.42, 109.21, 111.86, 115.65, 119.09, 123.51, 125.25, 126.80, 127.89, 131.13, 132.31, 133.76, 136.13, 140.22, 142.90, 146.56, 147.02, 155.42, 197.60

### Example 85b

¹H-NMR (DMSO-d₆, 200 MHz) δ(ppm):

2.31 (3H, s, 5′-CH₃), 2.60 (3H, s, 5-COCH₃), 5.79 (2H, s, —CH₂), 6.29 (1H, d, J=2.6 Hz, H-4′), 6.91˜6.95 (1H, m, H-5″), 7.04 (1H, d, J=3.34 Hz, H-3′), 7.18 (1H, s, 2″-CH₃), 7.73 (1H, d, J=8.58 Hz, H-7), 7.83˜7.88 (1H, m, H-6), 8.27 (1H, d, J=0.90 Hz, H-4)

¹³C-NMR (DMSO-d₆, 50 MHz) δ(ppm):

13.78, 27.23, 47.57, 109.09, 110.84, 115.17, 120.56 123.48, 125.32, 126.84, 127.96, 131.13, 132.62, 133.78, 139.50, 139.98, 142.74, 142.83, 146.11, 155.15, 197.77

### Example 86: Synthesis of 6-acetyl-1-(2-chlorobenzyl)-2-(5-methyl-2-furyl)benzimidazole (Example 86a) and 5-acetyl-1-(2-chlorobenzyl)-2-(5-methyl-2-furyl)benzimidazole (Example 86b)

6-Acetyl-1-(2-chlorobenzyl)-2-(5-methyl-2-furyl)benzimidazole (Example 86a) and 5-acetyl-1-(2-chlorobenzyl)-2-(5-methyl-2-furyl)benzimidazole (Example 86b) was prepared as in Example 25 at a yield of Example 86a (0.23 g, 29.0%) and Example 86b (0.11 g, 14.0%)

### Example 86a

¹H-NMR (DMSO-d₆, 200 MHz) δ(ppm):

2.22 (3H, s, 5′-CH₃), 2.55 (3H, s, 6-COCH₃), 5.88 (2H, s, —CH₂), 6.24 (1H, dd, J=0.92, 3.34 Hz, H-4′), 6.33 (1H, dd, J=1.32, 7.60 Hz, H-3″), 6.95 (1H, d, J=3.38 Hz, H-3′), 7.10˜7.25 (2H, m, H-4″, 5″), 7.50 (1H, dd, J=1.18, 6.70 Hz, H-4), 7.68 (1H, d, J=8.50 Hz, H-5), 7.83 (1H, dd, J=1.50, 8.52 Hz, H-6″), 8.24 (1H, d, J=0.94 Hz, H-4)

¹³C-NMR (DMSO-d₆, 50 MHz) δ(ppm):

13.66, 27.25, 46.15, 109.16, 111.97, 115.37, 119.12, 123.38, 126.76, 128.18, 129.50, 129.97, 131.78, 132.31, 135.14, 136.27, 143.04, 146.52, 147.07

### Example 86b

¹H-NMR (DMSO-d₆, 200 MHz) δ(ppm):

2.23 (3H, s, 5′-CH₃), 2.60 (3H, s, 5-COCH₃), 5.82 (2H, s, —CH₂), 6.25 (1H, dd, J=0.95, 3.36 Hz, H-4′), 6.25 (1H, dd, J=1.30, 7.40 Hz, H-3″), 6.29 (1H, d, J=3.20 Hz, H-3′), 7.07˜7.29 (2H, m, H-4″, 5″), 7.50 (1H, dd, J=1.20, 8.00 Hz, H-7), 7.61 (1H, d, J=8.60 Hz, H-6), 7.81 (1H, dd, J=1.60, 8.60 Hz, H-6″), 8.29 (1H, d, J=1.20 Hz, H-4)

¹³C-NMR (DMSO-d₆, 50 MHz) δ(ppm):

13.69, 46.27, 109.07, 110.78, 114.92, 120.57, 123.53, 126.81, 128.19, 129.60, 130.03, 131.77, 132.64, 134.84, 139.70, 142.70, 142.91, 146.22, 155.04, 197.82,

### Example 87: Synthesis of 6-acetyl-1-(4-chlorobenzyl)-2-(5-methyl-2-furyl)benzimidazole (Example 87a) and 5-acetyl-1-(4-chlorobenzyl)-2-(5-methyl-2-furyl)benzimidazole (Example 87b)

6-Acetyl-1-(4-chlorobenzyl)-2-(5-methyl-2-furyl)benzimidazole (Example 87a) and 5-acetyl-1-(4-chlorobenzyl)-2-(5-methyl-2-furyl)benzimidazole (Example 87b) was prepared as in Example 25 at a yield of Example 87a (0.15 g, 19.0%) and Example 87b (0.16 g, 20.0%)

### Example 87a

¹H-NMR (DMSO-d₆, 400 MHz) δ(ppm):

2.36 (3H, s, 5′-CH₃), 2.63 (3H, s, 6-COCH₃), 5.90 (2H, s, —CH₂), 6.34 (1H, d, J=2.80 Hz, H-4′), 7.11˜7.14 (3H, m, H-3′, 2″, 6″), 7.12 (2H, d, J=8.40 Hz, H-3″, 5″), 7.73 (1H, d, J=8.40 Hz, H-4), 7.88 (1H, m, H-5), 8.29 (1H, s, H-7)

¹³C-NMR (DMSO-d₆, 100 MHz) δ(ppm):

13.84, 27.28, 47.35, 109.20, 111.90, 115.64, 119.07, 123.47, 128.63, 129.19, 132.25, 132.51, 136.11, 136.66, 142.94, 146.59, 147.02, 155.44, 197.61

### Example 87b

¹H-NMR (DMSO-d₆, 400 MHz) δ(ppm):

2.36 (3H, s, 5′-CH₃), 2.64 (3H, s, 5-COCH₃), 5.82 (2H, s, —CH₂), 6.34 (1H, d, J=2.80 Hz, H-4′), 7.08˜7.13 (3H, m, H-3′, 2″, 6″), 7.12 (2H, d, J=8.40 Hz, H-3″, 5″), 7.37 (1H, d, J=8.40 Hz, H-7), 7.88 (1H, d, J=8.40 Hz, H-6), 8.31 (1H, s, H-4)

¹³C-NMR (DMSO-d₆, 100 MHz) δ(ppm):

13.75, 27.17, 47.48, 109.01, 110.81, 115.10, 120.49, 123.36, 128.62, 129.13, 132.53, 136.37, 139.44, 142.72, 142.83, 146.07, 155.10, 197.13

### Example 88: Synthesis of 6-acetyl-1-(4-fluorobenzyl)-2-(5-methyl-2-furyl)benzimidazole (Example 88a) and 5-acetyl-1-(4-fluorobenzyl)-2-(5-methyl-2-furyl)benzimidazole (Example 88b)

6-Acetyl-1-(4-fluorobenzyl)-2-(5-methyl-2-furyl)benzimidazole (Example 88a) and 5-acetyl-1-(4-fluorobenzyl)-2-(5-methyl-2-furyl)benzimidazole (Example 88b) was prepared as in Example 25 at a yield of Example 88a (0.16 g, 22.0%) and Example 88b (0.13 g, 18.0%)

### Example 88a

¹H-NMR (DMSO-d₆, 200 MHz) δ(ppm):

2.37 (3H, s, 5′-CH₃), 2.63 (3H, s, 6-COCH₃), 5.89 (2H, s, —CH₂), 6.35 (1H, s, H-4′), 7.13˜7.17 (5H, m, H-3′, 2″, 3″, 5″, 6″), 7.74 (1H, d, J=8.40 Hz, H-4), 7.88 (1H, d, J=8.40 Hz, H-5), 8.30 (1H, s, H-7)

¹³C-NMR (DMSO-d₆, 50 MHz) δ(ppm):

13.79, 27.24, 47.24, 109.15, 111.91, 115.60, 115.88, 116.10, 119.01, 123.38, 128.79, 128.88, 133.72, 133.75, 136.04, 142.94, 146.56, 146.96, 155.38, 197.59

### Example 88b

¹H-NMR (DMSO-d₆, 200 MHz) δ(ppm):

2.37 (3H, s, 5′-CH₃), 2.64 (3H, s, 5-COCH₃), 5.81 (2H, s, —CH₂), 6.35 (1H, s, H-4′), 7.10˜7.16 (5H, m, H-3′, 2″, 3″, 5″, 6″), 7.88 (1H, d, J=3.60 Hz, H-7), 7.88 (1H, d, J=3.40 Hz, H-6), 8.31 (1H, s, H-4)

¹³C-NMR (DMSO-d₆, 50 MHz) δ(ppm):

13.79, 27.22, 47.40, 109.03, 110.92, 115.12, 115.88, 116.10, 120.48, 123.34, 128.84, 128.92, 132.50, 139.44, 142.72, 142.86, 155.11, 197.79

### Example 89: Synthesis of 6-acetyl-1-(4-methylbenzyl)-2-(5-methyl-2-furyl)benzimidazole (Example 89a) and 5-acetyl-1-(4-methylbenzyl)-2-(5-methyl-2-furyl)benzimidazole (Example 89b)

6-Acetyl-1-(4-methylbenzyl)-2-(5-methyl-2-furyl)benzimidazole (Example 89a) and 5-acetyl-1-(4-methylbenzyl)-2-(5-methyl-2-furyl)benzimidazole (Example 89b) was prepared as in Example 25 at a yield of Example 89a (0.19 g, 26.0%) and Example 89b (0.14 g, 18.0%)

### Example 89a

¹H-NMR (DMSO-d₆, 400 MHz) δ(ppm):

2.22 (3H, s, 4″-CH₃), 2.37 (3H, s, 5′-CH₃), 2.62 (3H, s, 6-COCH₃), 5.84 (2H, s, —CH₂), 6.35 (1H, d, J=2.80 Hz, H-4′), 7.01 (2H, d, J=8.00 Hz, H-2″, 6″), 7.09˜7.12 (3H, m, H-3′, 3″, 5″), 7.74 (1H, d, J=8.40 Hz, H-4), 7.89 (1H, m, H-5), 8.26 (1H, s, H-7)

¹³C-NMR (DMSO-d₆, 100 MHz) δ(ppm):

13.82, 20.99, 27.24, 47.67, 109.13, 111.94, 115.54, 118.97, 123.33, 126.67, 129.70, 132.10, 134.42, 136.12, 137.10, 142.99, 146.57, 147.05, 155.30, 197.58

### Example 89b

¹H-NMR (DMSO-d₆, 400 MHz) δ(ppm):

2.20 (3H, s, 4″-CH₃), 2.36 (3H, s, 5′-CH₃), 2.63 (3H, s, 5-COCH₃), 5.75 (2H, s, —CH₂), 6.33 (1H, s, H-4′), 6.97˜7.29 (5H, m, H-3′, 2″, 3″, 5″, 6″), 7.68 (1H, d, J=8.0 Hz, H-7), 7.86 (1H, d, J=8.40 Hz, H-6), 8.30 (1H, s, H-4)

¹³C-NMR (DMSO-d₆, 100 MHz) δ(ppm):

13.77, 20.95, 27.16, 47.84, 108.98, 110.92, 115.03, 120.46, 123.24, 126.71, 129.68, 132.41, 134.23, 137.16, 139.51, 142.73, 142.95, 146.16, 155.00, 197.73.

### Example 90-113: Synthesis of 1-substituted benzyl-2-(5-methyl-2-thienyl)benzoimidazoles

o-Phenylenediamine (10.8 g, 0.1 mol) and 5-methyl-2-thiophenecarboxaldehyde (12.6 g, 0.1 mol) were mixed in DMF (10 ml), then sodium metabisulfite (19 g, 0.1 mol) was added, and the solution was heated and stirred at 100° C. for 3 h. When the reaction was completed, the reaction mixture was cooled and added dropwise with vigorous stirring onto a mixture of anhydrous sodium carbonate (10.6 g, 0.1 mol) and distilled water (20 ml). The product 2-(5-Methyl-2thienyl)benzimidazole was collected by filtration, washed with H2O and dried.

2-(5-Methyl-2-furyl)benzimidazole (0.2 g, 1.0 mmol) was mixed with potassium carbonate (0.7 g, 5 mmol) in 95% ethanol (50 ml) and heated to boiling. Alkyl chloride or substituted benzyl chloride (5 mmol) was added drop wise with vigorous stirring of the mixture. After complete addition of the Alkyl chloride or substituted benzyl chloride and reflux for about 4 hours (h). The reaction mixture was cooled and poured into water. The aqueous solution was extracted with dichloromethane, and the dichloromethane extracts were washed with water and dried over sodium sulfate. Evaporation of the solvent gave an oil that was purified by chromatography on a silica gel column using dichloromethane as eluent affording the expected products.

### Example 90: 1-benzyl-2-(5-methyl-2-thienyl)benzoimidazole

¹H-NMR (CDCl₃, 200 MHz) δ(ppm)

2.18 (3H, s, 5′-CH₃), 5.54 (2H, s-CH₂), 6.68 (1H, d, J=2.8, H-4′), 7.07˜7.30 (9H, m, H-2″, 3″, 4″, 5″, 6″, 3′, 5, 6, 7), 7.81 (1H, d, J=7.5, H-4)

¹³C-NMR (CDCl₃, 500 MHz) δ(ppm)

15.35, 48.16, 109.88, 11966, 122.83, 123.04, 125.86 (d), 126.28, 127.80, 128.13, 129.11 (d), 129.44, 136.11, 136.25, 142.94, 143.86, 148.27

### Example 91: 1-(4-chlorobenzyl)-2-(5-methyl-2-thienyl)benzoimidazole

¹H-NMR (CDCl₃, 400 MHz) δ(ppm)

2.55 (3H, s, 5′-CH₃), 5.57 (2H, s-CH₂), 6.76 (1H, d, J=2.8, H-4′), 7.07 (2H, d, J=8, H-2″, 6″), 7.11 (1H, d, J=3.6, H-7), 7.25 (1H, s, H-6), 7.28˜7.34 (3H, m, H-3′, 3″, 5″), 7.85 (1H, d, J=8, H-4)

¹³C-NMR (CDCl₃, 100 MHz) δ(ppm)

15.30, 47.56, 109.62, 119.77, 123.02 (d), 126.22, 127.22, 128.06, 128.55, 129.27, 133.67, 134.58, 135.99, 142.97, 143.96, 148.15

### Example 92: 1-(3-chlorobenzyl)-2-(5-methyl-2-thienyl)benzoimidazole

¹H-NMR (CDCl₃, 400 MHz) δ(ppm)

2.50 (3H, s, 5′-CH₃), 5.52 (2H, s-CH₂), 6.72 (1H, d, J=3.6, H-4′), 6.93 (1H, d, J=6.8, H-6), 7.06 (1H, d, J=3.6, H-7), 7.13 (1H, s, H-5), 7.15˜7.21 (2H, m, H-2″, 6″), 7.24 (2H, d, J=5.2, H-3″4″), 7.30 (1H, s, H-3′), 7.81 (1H, d, J=8, H-4)

¹³C-NMR (CDCl₃, 100 MHz) δ(ppm)

14.80, 47.12, 109.10, 119.30, 122.44, 122.65, 123.44, 125.52, 125.75, 127.51, 127.59, 128.73, 129.91, 134.62, 135.52, 137.71, 142.52, 143.46, 147.68

### Example 93: 1-(2-chlorobenzyl)-2-(5-methyl-2-thienyl)benzoimidazole

¹H-NMR (CDCl₃, 400 MHz) δ(ppm)

2.49 (3H, s, 5′-CH₃), 5.60 (2H, s-CH₂), 6.62 (1H, d, J=8, H-4′), 6.63 (1H, s, H-5), 6.69 (1H, d, J=1.2, H-7), 7.09˜7.20 (2H, m, H-2″, 6″), 7.21 (2H, d, J=1.2, H-3″5″), 7.28 (1H, t, J=4, 8, H-3′), 7.47 (1H, d, J=7.2, 8, H-6), 7.83 (1H, d, J=8, H-4)

¹³C-NMR (CDCl₃, 100 MHz) δ(ppm)

15.29, 46.18, 109.52, 119.77, 122.94, 123.11, 126.32, 126.77, 127.50, 127.77, 129.00, 129.30, 129.72, 131.97, 133.52, 136.08, 143.03, 143.91, 148.29

### Example 94: 1-(4-methylbenzyl)-2-(5-methyl-2-thienyl)benzoimidazole

¹H-NMR (CDCl₃, 400 MHz) δ(ppm)

2.34 (3H, s, 4″-CH₃), 2.54 (3H, s, 5′-CH₃), 5.56 (2H, s-CH₂), 6.74 (1H, d, J=3.2, H-4′), 7.01 (1H, s, H-7), 7.03 (1H, s, H-5), 7.15 (2H, d, J=8.4, H-2″, 6″), 7.23 (2H, d, J=4, H-3″5″), 7.30˜7.32 (1H, m, H-3′), 7.85 (1H, d, J=8, H-4)

¹³C-NMR (CDCl₃, 100 MHz) δ(ppm)

15.30, 21.02, 47.94, 109.86, 119.59, 122.70, 122.92, 125.74, 126.19, 128.05, 129.49, 129.71, 133.03, 136.23, 137.47, 142.94, 143.72, 148.24

### Example 95: 1-(3-methylbenzyl)-2-(5-methyl-2-thienyl)benzoimidazole

¹H-NMR (CDCl₃, 400 MHz) δ(ppm)

2.30 (3H, s, 4″-CH₃), 2.53 (3H, s, 5′-CH₃), 5.56 (2H, s-CH₂), 6.74 (1H, d, J=2, H-4′), 6.91 (1H, s, H-7), 6.94 (1H, s, H-5), 7.10˜7.14 (2H, m, H-2″, 6″),

7.21˜7.25 (2H, m, H-3″4″), 7.27˜7.29 (1H, m, H-3′), 7.85 (1H, d, J=8, H-4′)

¹³C-NMR (CDCl₃, 100 MHz) δ(ppm)

15.30, 21.40, 48.13, 109.87, 119.57, 122.75, 122.88, 122.96, 126.23, 126.35, 128.09, 128.52, 128.94, 129.43, 136.02, 136.25, 138.88, 142.88, 143.79, 148.27

### Example 96: 1-(4-methoxylbenzyl)-2-(5-methyl-2-thienyl)benzoimidazole

¹H-NMR (CDCl₃, 400 MHz) δ(ppm)

2.54 (3H, s, 4″-CH₃), 3.78 (3H, s, 5′-CH₃), 5.53 (2H, s-CH₂), 6.74 (1H, d, J=2.8, H-4′), 6.86 (2H, d, J=8.8, H-5, 7), 7.05 (2H, d, J=8.8, H-2″, 6″), 7.16 (1H, d, J=3.6, H-6), 7.23 (2H, d, J=4, H-3″, 5″), 7.27˜7.29 (1H, m, H-3′), 7.83 (1H, d, J=8, H-4)

¹³C-NMR (CDCl₃, 100 MHz) δ(ppm)

15.30, 47.64, 55.23, 109.87, 114.42, 119.59, 122.71, 122.93, 126.19, 127.08, 128.00, 128.10, 128.10, 129.44, 136.17, 142.90, 143.75, 148.17, 159.10

### Example 97: 1-(3-methoxylbenzyl)-2-(5-methyl-2-thienyl)benzoimidazole

¹H-NMR (CDCl₃, 400 MHz) δ(ppm)

2.53 (3H, s, 4″-OCH₃), 3.73 (3H, s, 5′-CH₃), 5.54 (2H, s-CH₂), 6.74 (1H, d, J=2, H-4′), 6.68˜6.71 (2H, m, H-2″, 6″), 6.74 (1H, d, J=1.2, 4, H-5), 6.84 (1H, d, J=2, 8, H-7), 7.23 (2H, d, J=3.6, H- 3″, 5″), 7.28˜7.31 (2H, m, H-3′, 6), 7.83 (1H, d, J=4, H-4)

¹³C-NMR (CDCl₃, 100 MHz) δ(ppm)

15.10, 47.86, 54.97, 109.62, 111.55, 112.67, 117.85, 119.47, 122.55, 122.78, 126.03, 127.83, 129.34, 129.99, 136.09, 137.61, 142.83, 143.54, 148.08, 160.00

### Example 98: 1-(2-methoxylbenzyl)-2-(5-methyl-2-thienyl)benzoimidazole

¹H-NMR (CDCl₃, 400 MHz) δ(ppm)

2.53 (3H, s, 4″-OCH₃), 3.73 (3H, s, 5′-CH₃), 5.54 (2H, s-CH₂), 6.74 (1H, d, J=2, H-4′), 6.68˜6.71 (2H, m, H-2″, 6″), 6.74 (1H, d, J=1, 2, 4, H-5), 6.84 (1H, d, J=2, 8, H-7), 7.23 (2H, d, J=3.6, H- 3″, 4″), 7.28˜7.31 (2H, m, H-3′, 6), 7.83 (1H, d, J=4, H-4)

¹³C-NMR (CDCl₃, 100 MHz) δ(ppm)

15.10, 47.86, 54.97, 109.62, 111.55, 112.67, 117.85, 119.47, 122.55, 122.78, 126.03, 127.83, 129.34, 129.99, 136.09, 137.61, 142.83, 143.54, 148.08, 160.00

### Example 99: 1-(4-fluorobenzyl)-2-(5-methyl-2-thienyl)benzoimidazole

¹H-NMR (CDCl₃, 400 MHz) δ(ppm) 2.49 (3H, s, 4″-OCH₃), 3.91 (3H, s, 5′-CH₃), 5.53 (2H, s-CH₂), 6.60 (1H, d, J=6.8, H-4′), 6.61 (1H, s, H-5), 6.93˜7.02 (2H, m, H-2″, 6″), 7.18 (2H, d, J=4, H-3″, 5″), 7.25 (1H, s, H-6), 7.26˜7271 (1H, m, H-3′), 7.81 (1H, d, J=8, H-4)

¹³C-NMR (CDCl₃, 100 MHz) δ(ppm)

15.56, 44.06, 55.61, 110.11, 110.34, 119.83, 121.14, 122.88, 123.07, 124.48, 126.46, 126.54, 128.10, 128.96, 129.96, 136.63, 143.27, 143.81, 148.73, 156.53

### Example 100: 1-(3-fluorobenzyl)-2-(5-methyl-2-thienyl)benzoimidazole

¹H-NMR (CDCl₃, 400 MHz) δ(ppm)

2.55 (3H, s, 5′-CH₃), 5.60 (2H, s-CH₂), 6.77 (1H, d, J=2.4, H-4′), 6.86 (1H, d, J=9.6, H-7), 6.92 (1H, d, J=7.6, H-5), 7.02 (1H, t, J=8, H-3′), 7.22˜7.24 (2H, m, H-2″, 6″), 7.25˜7.36 (2H, m, H-3″, 4″), 7.86 (1H, d, J=8, H-4)

¹³C-NMR (CDCl₃, 100 MHz) δ(ppm)

15.30, 47.68, 109.61, 112.90, 113.12, 114.83 (d), 119.77, 121.39, 122.93, 123.14, 126.23, 128.02, 129.23, 130.77 (d), 136.08, 138.78, 142.98, 143.96, 148.15

### Example 101: 1-(2-fluorobenzyl)-2-(5-methyl-2-thienyl)benzoimidazole

2.55 (3H, s, 5′-CH₃), 5.60 (2H, s-CH₂), 6.77 (1H, d, J=2.4, H-4′), 6.86 (1H, d, J=9.6, H-7), 6.92 (1H, d, J=7.6, H-5), 7.02 (1H, t, J=8, H-3′), 7.22˜7.24 (2H, m, H-2″, 6″), 7.25˜7.36 (2H, m, H-3″, 5″), 7.86 (1H, d, J=8, H-4)

¹³C-NMR (CDCl₃, 100 MHz) δ(ppm)

15.30, 47.68, 109.61, 112.90, 113.12, 114.83 (d), 119.77, 121.39, 122.93, 123.14, 126.23, 128.02, 129.23, 130.77 (d), 136.08, 138.78, 142.98, 143.96, 148.15

### Example 102a: 1-(4-chlorobenzyl)-6-fluoro-2-(5-methyl-2-thienyl)benzimidazole

¹H-NMR (CDCl₃, 400 MHz) δ(ppm)

2.50 (3H, s, 5′-CH₃), 5.50 (2H, s-CH₂), 6.72 (1H, s, H-4′), 6.92˜6.94 (1H, m, H-3′), 6.97 (2H, d, J=2, H- 2″, 6″), 7.03 (2H, d, J=5.2, H-5, 7), 7.29 (2H, d, J=8.4, H-3″, 5″), 7.46 (1H, dd, J=2, 9.2, H-4)

¹³C-NMR (CDCl₃, 100 MHz) δ(ppm)

15.29, 47.74, 96.49, 111.23, 120.52, 126.25, 127.18, 128.64, 128.90, 133.97, 134.07, 136.09, 144.01, 148.87, 158.61, 161.01

### Example 102b: 1-(4-chlorobenzyl)-5-fluoro-2-(5-methyl-2-thienyl)benzimidazole

¹H-NMR (CDCl₃, 400 MHz) δ(ppm)

2.50 (3H, s, 5′-CH₃), 5.50 (2H, s-CH₂), 6.72 (1H, s, H-4′), 6.92˜6.94 (1H, m, H-3′), 6.97 (2H, d, J=2, H- 2″, 6″), 7.03 (2H, d, J=5.2, H-5, 7), 7.29 (2H, d, J=8.4, H-3″, 5″), 7.46 (1H, dd, J=2, 9.2, H-4)

¹³C-NMR (CDCl₃, 100 MHz) δ(ppm)

15.30, 47.74, 105.54, 109.94, 111.32, 126.30, 127.16, 128.20, 128.89, 129.35, 132.51, 133.83, 134.25, 143.47, 144.31, 149.56, 160.93

### Example 103a: 1-(3-chlorobenzyl)-6-fluoro-2-(5-methyl-2-thienyl)benzimidazole

¹H-NMR (CDCl₃, 400 MHz) δ(ppm)

2.51 (3H, s, 5′-CH₃), 5.51 (2H, s, —CH₂), 6.72 (1H, d, J=2, H-4′), 6.85 (1H, d, J=2, H-7), 6.93 (1H, d, J=5.6, H-3′), 7.05 (2H, d, J=4, H-2″, 6″), 7.17 (2H, d, J=7.6, H-3″, 4″), 7.72 (1H, dd, J=4.8, 8.8, H-4)

¹³C-NMR (CDCl₃, 100 MHz) δ(ppm)

15.29, 47.79, 96.50 (d), 111.24, 120.52 (d), 123.88, 124.75, 125.97, 126.29, 126.86, 127.47, 128.17 (d), 128.79, 129.67, 130.50, 135.23, 139.67, 139.32, 144.08

### Example 103b: 1-(3-chlorobenzyl)-5-fluoro-2-(5-methyl-2-thienyl)benzimidazole

¹H-NMR (CDCl₃, 400 MHz) δ(ppm)

2.54 (3H, s, 5′-CH₃), 5.54 (2H, s-CH₂), 6.76 (1H, d, J=3.2, H-4′), 6.96 (1H, d, J=6.8, H-5), 6.99 (1H, d, J=2, H-7), 7.09 (2H, dd, J=8.4, 4.8, H-2″, 6″), 7.14 (1H, s, H-3′), 7.27 (2H, d, J=7.6, H-3″, 4″) 7.50 (1H, dd, J=2.4, 9.6, H-4)

¹³C-NMR (CDCl₃, 100 MHz) δ(ppm)

15.30, 47.78, 105.54 (d), 109.9 (d), 111.36 (d), 123.87, 125.97, 126.35, 128.21, 128.87, 130.48, 132.52, 135.20, 137.86, 143.43, 144.34, 149.85, 158.58, 160.95

### Example 104b: 1-(3-chlorobenzyl)-5-fluoro-2-(5-methyl-2-thienyl)benzimidazole

¹H-NMR (CDCl₃, 400 MHz) δ(ppm)

2.49 (3H, s, 5′-CH₃), 5.58 (2H, s, —CH₂), 6.60 (1H, d, J=1.2, H-4′), 6.61 (1H, dd, J=8, 1.2, H-4) 6.62 (1H, d, J=0.8, H-3′), 6.95˜6.98 (2H, m, J=5.6, H-2″, 6″), 7.04˜7.08 (1H, m, J=4, H-5), 7.11 (1H, t, H-7), 7.13˜7.25 (2H, m, H-3″, 5″)

¹³C-NMR (CDCl₃, 100 MHz) δ(ppm)

15.29, 47.79, 96.50 (d), 111.24, 120.52 (d), 123.88, 124.75, 125.97, 126.29, 126.86, 127.47, 128.17 (d), 128.79, 129.67, 130.50, 135.23, 139.67, 139.32, 144.08

### Example 105a: 1-(4-methylbenzyl)-6-fluoro-2-(5-methyl-2-thienyl)benzimidazole

¹H-NMR (CDCl₃, 400 MHz) δ(ppm)

2.31 (3H, s, 4″-CH₃), 2.49 (3H, s, 5′-CH₃), 5.46 (2H, s, —CH₂), 6.69˜6.70 (1H, m, H-4′), 6.85 (1H, d, J=8.8, H-7) 6.96 (1H, s, H-5), 6.97˜6.98 (2H, m, H-2″, 6″), 7.08 (1H, d, J=3.6, H-3′), 7.12 (2H, d, J=8, H-3″, 5″), 7.70 (1H, dd, J=4.8, 8.8, H-4)

¹³C-NMR (CDCl₃, 100 MHz) δ(ppm)

15.28, 21.02, 48.14, 96.69 (d), 110.99 (d), 120.26, 120.31 (d), 126.21, 128.03, 129.20, 129.80, 132.52, 136.38 (d), 137.68, 139.37, 143.7, 148.99, 160.92

### Example 105b: 1-(4-methylbenzyl)-5-fluoro-2-(5-methyl-2-thienyl)benzimidazole

¹H-NMR (CDCl₃, 400 MHz) δ(ppm)

2.30 (3H, s, 4″-CH₃), 2.50 (3H, s, 5′-CH₃), 5.50 (2H, s, —CH₂), 6.71 (1H, d, J=2.8, H-4′), 6.91 (1H, dd, J=2.4, H-7) 6.93 (1H, d, J=2.4, H-5), 6.96 (2H, d, J=8.4, H-2″, 6″), 7.06˜7.07 (1H, m, J=4, H-3′), 7.11˜7.15 (2H, m, H-3″, 5″), 7.46 (1H, dd, J=2.4, 9.6, H-4)

¹³C-NMR (CDCl₃, 100 MHz) δ(ppm)

15.31, 21.02, 48.16, 105.24 (d), 110.25 (d), 111.08, 111.34, 125.70, 126.34, 127.83, 128.46, 128.93 (d), 129.79, 132.60, 137.68, 144.25, 149.51, 158.52, 160.89

### Example 106a: 1-(2-methylbenzyl)-6-fluoro-2-(5-methyl-2-thienyl)benzimidazole

¹H-NMR (CDCl₃, 400 MHz) δ(ppm)

2.33 (3H, s, 2″-CH₃), 2.48 (3H, s, 5′-CH₃), 5.41 (2H, s, —CH₂), 6.67 (1H, d, J=2.8, H-4′), 6.79 (1H, d, J=2, H-5) 7.01 (2H, dd, J=7.2, 8.8, H-2″, 6″), 7.07 (1H, s, H-3′), 7.21 (2H, d, J=7.2, H-3″, 5″), 7.73 (1H, dd, J=4.8, 8.8, H-4)

¹³C-NMR (CDCl₃, 100 MHz) δ(ppm)

15.28, 19.08, 46.59, 96.55 (d), 109.72, 111.08 (d), 119.67, 120.39, (d), 124.78, 126.27, 126.77, 127.80 (d), 129.16, 130.61, 133.41, 134.44, 139.38, 143.83, 149.13, 158.58

### Example 106b: 1-(2-methylbenzyl)-5-fluoro-2-(5-methyl-2-thienyl)benzimidazole

¹H-NMR (CDCl₃, 400 MHz) δ(ppm)

2.44 (3H, s, 2″-CH₃), 2.52 (3H, s, 5′-CH₃), 5.47 (2H, s, —CH₂), 6.61 (1H, d, J=7.6, H-4′), 6.71 (1H, d, J=3.2, H-7) 6.94˜6.97 (3H, m, H-2″, 5, 6″), 7.05˜7.08 (3H, m, H-3′, 3″, 5′), 7.52 (1H, dd, J=2, 9.2, H-4)

¹³C-NMR (CDCl₃, 100 MHz) δ(ppm)

15.29, 19.08, 46.58, 105.38 (d), 114.14 (d), 124.79, 126.34, 126.75, 127.71, 128, 129.14, 130.59, 132.80, 133.60, 134.42, 143.47 (d), 144.13, 149.81, 158.32, 160.89

### Example 107a: 1-(4-methoxylbenzyl)-6-fluoro-2-(5-methyl-2-thienyl)benzimidazole

¹H-NMR (CDCl₃, 400 MHz) δ(ppm)

2.52 (3H, s, 4″-CH₃), 3.78 (3H, s, 5′-CH₃), 5.47 (2H, s, —CH₂), 6.73 (1H, s, H-4′), 6.85˜6.90 (3H, m, H- 2″, 5, 6″), 6.98˜7.04 (2H, m, H-3″, 5″), 7.26 (1H, s, H-3′) 7.71 (1H, dd, J=4.8, 8.8, H-4)

¹³C-NMR (CDCl₃, 100 MHz) δ(ppm)

15.28, 47.86, 55.24, 96.71 (d), 111.01 (d), 114.32, 120.31 (d), 126.21, 127.27 (d), 128.07, 136.27, 139.36, 143.79, 148.95, 159.23

### Example 107b: 1-(4-methoxylbenzyl)-5-fluoro-2-(5-methyl-2-thienyl)benzimidazole

¹H-NMR (CDCl₃, 400 MHz) δ(ppm)

2.52 (3H, s, 4″-CH₃), 3.77 (3H, s, 5′-CH₃), 5.50 (2H, s, —CH₂), 6.74 (1H, d, J=2.4, H-4′), 6.86 (2H, d, J=8.4, H-2″, 6″), 6.93˜6.96 (1H, m, H-5), 7.02 (2H, d, J=8.4, H-3″, 5″), 7.26 (1H, s, H-3′) 7.47 (1H, dd, J=2, 9.2, H-4)

¹³C-NMR (CDCl₃, 100 MHz) δ(ppm)

15.30, 47.84, 55.24, 105.34 (d), 110.23, 111.01 (d), 114.49, 126.26, 127.04, 127.67, 128.19, 129.20, 132.70, 144.06, 159.16

### Example 108a: 1-(3-methoxylbenzyl)-6-fluoro-2-(5-methyl-2-thienyl)benzimidazole

¹H-NMR (CDCl₃, 400 MHz) δ(ppm)

2.50 (3H, s, 3″-OCH₃), 3.71 (3H, s, 5′-CH₃), 5.47 (2H, s, —CH₂), 6.65 (1H, s, H-4′), 6.67 (1H, s, H-7), 6.71 (1H, d, J=4, H-5), 6.08˜6.88 (2H, m, H-2″, 6″), 6.99 (1H, ddd, J=8.4, 2, H-3′) 7.09˜7.26 (1H, dd, J=8.8, 8, H-4)

¹³C-NMR (CDCl₃, 100 MHz) δ(ppm)

15.29, 48.26, 55.18, 96.67 (d), 109.91, 111.23 (d), 112.35 (d), 117.97, 119.35, 120.32 (d), 126.33 (d), 128.21, 128.96, 130.29, 136.28, 137.19, 139.17, 143.94, 148.95, 160.24

### Example 108b: 1-(3-methoxylbenzyl)-5-fluoro-2-(5-methyl-2-thienyl)benzimidazole

¹H-NMR (CDCl₃, 400 MHz) δ(ppm)

2.50 (3H, s, 3″-OCH₃), 3.71 (3H, s, 5′-CH₃), 5.50 (2H, s, —CH₂), 6.618 (1H, s, H-4′), 6.61 (1H, s, H-4′), 6.65 (1H, d, J=7.6, H-3′), 7.08˜7.14 (2H, m, H-2″, 6″), 7.24 (1H, t, H-3′) 7.46 (1H, d, J=9.2, H-4)

¹³C-NMR (CDCl₃, 100 MHz) δ(ppm)

15.31, 48.27, 55.17, 105.18, 105.43, 110.19, 111.15, 111.41, 111.72, 112.94, 117.94, 126.36, 128.48, 128.81, 130.27, 132.65, 137.33, 144.31, 149.54, 160.23

### Example 109a: 1-(4-fluorolbenzyl)-6-fluoro-2-(5-methyl-2-thienyl)benzimidazole

¹H-NMR (CDCl₃, 400 MHz) δ(ppm)

2.50 (3H, s, 5′-CH₃), 5.48 (2H, s, —CH₂), 6.71 (1H, s, H-4′), 6.84 (1H, d, J=2, H-3′), 6.98˜6.99 (2H, m, H-3″, 5″) 7.01˜7.45 (2H, m, H-2″, 6″), 7.09˜7.11 (2H, m, H-5, 7), 7.71 (1H, dd, J=4.8, 8.8, H-4)

¹³C-NMR (CDCl₃, 100 MHz) δ(ppm)

15.29, 47.73, 96.45, 96.73, 111.17, 111.41, 116.07, 116.28, 120.37, 120.47, 126.27, 127.49, 128.22, 131.21, 136.05, 144.11

### Example 109b: 1-(4-fluorolbenzyl)-5-fluoro-2-(5-methyl-2-thienyl)benzimidazole

¹H-NMR (CDCl₃, 400 MHz) δ(ppm)

2.51 (3H, s, 5′-CH₃), 5.52 (2H, s, —CH₂), 6.73 (1H, s, H-4′), 6.93˜6.95 (2H, m, H-3″, 5″), 7.03˜7.05 (2H, m, H-2″, 6″), 7.06˜7.09 (2H, m, H-5, 7), 7.15 (1H, s, H-3′), 7.48 (1H, dd, J=1.6, 9.2, H-4)

¹³C-NMR (CDCl₃, 100 MHz) δ(ppm)

15.31, 47.75, 105.26, 105.51, 110.10 (d), 111.32, 111.58, 116.05, 116.27, 126.39, 127.46, 127.54, 128.59, 131.29, 132.37, 144.54, 149.37

### Example 110a: 1-(3-fluorolbenzyl)-6-fluoro-2-(5-methyl-2-thienyl)benzimidazole

¹H-NMR (CDCl₃, 400 MHz) δ(ppm)

2.50 (3H, s, 5′-CH₃), 5.49 (2H, s, —CH₂), 6.71 (1H, s, H-4′), 6.84 (1H, d, J=6.8, H-3″), 6.85˜6.87 (2H, m, H-3″, 4″), 6.97˜7.05 (2H, m, H-2″, 6″) 7.24˜7.29 (2H, m, H-5, 7), 7.72 (1H, dd, J=4.8, 8.8, H-4)

¹³C-NMR (CDCl₃, 100 MHz) δ(ppm)

15.28, 47.85, 39.35, 96.48 (d), 111.12, 111.37, 112.86, 113.09, 114.92, 115.13, 120.54 (d), 121.35, 126.25, 128.02, 128.91, 130.87, 138.25, 139.37, 144.01, 162.04

### Example 110b: 1-(3-fluorolbenzyl)-5-fluoro-2-(5-methyl-2-thienyl)benzimidazole

¹H-NMR (CDCl₃, 400 MHz) δ(ppm)

2.50 (3H, s, 5′-CH₃), 5.52 (2H, s, —CH₂), 6.72 (1H, d, J=2.8, H-4′), 6.79 (1H, d, J=9.2, H-3″), 6.85 (1H, d, J=7.6, H-4″), 6.93˜6.98 (2H, m, H-2″, 6″) 7.00˜7.05 (2H, m, H-5, 7), 7.24˜7.28 (1H, m, H-3″), 7.46 (1H, dd, J=2, 8.4, H-4)

¹³C-NMR (CDCl₃, 100 MHz) δ(ppm)

15.30, 47.86, 105.55 (d), 109.94 (d), 111.34 (d), 112.97 (d), 114.98 (d), 121.36, 126.32, 128.18, 128.91, 130.85 (d), 130.90, 132.56, 138.37, 143.54, 144.32, 149.58, 162.03, 164.49

### Example 111a: 1-(2-fluorolbenzyl)-6-fluoro-2-(5-methyl-2-thienyl)benzimidazole

¹H-NMR (CDCl₃, 400 MHz) δ(ppm)

2.49 (3H, s, 5′-CH₃), 5.55 (2H, s, —CH₂), 6.69˜6.72 (2H, m, H-5, 7), 6.87 (1H, d, J=8.4, H-4′), 6.98˜7.02 (2H, m, H-2″, 6″), 7.27˜7.28 (1H, m, H-5′), 7.72 (1H, dd, J=4, 8, H-4)

¹³C-NMR (CDCl₃, 100 MHz) δ(ppm) 1

15.18, 42.37, 96.35 (d), 111.10 (d), 115.53 (d), 116.06 (d), 120.40 (d), 122.78, 124.71, 126.19, 127.10, 127.86, 128.86, 129.85 (d), 136.20, 139.28, 143.85, 148.86, 158.51, 160.96

### Example 111b: 1-(2-fluorolbenzyl)-5-fluoro-2-(5-methyl-2-thienyl)benzimidazole

¹H-NMR (CDCl₃, 400 MHz) δ(ppm)

2.50 (3H, s, 5′-CH₃), 5.58 (2H, s, —CH₂), 6.69˜6.73 (2H, m, H-4′, 7), 6.94˜7.00 (2H, m, H-2″, 6″), 7.07˜7.16 (3H, m, H-3′, 3″, 5″), 7.24˜7.26 (1H, m, H-5), 7.45 (1H, dd, J=2.4, 9.2, H-4)

¹³C-NMR (CDCl₃, 100 MHz) δ(ppm)

14.96, 42.186, 105.17 (d), 109.57 (d), 110.96 (d), 115.25 (d), 122.65 (d), 124.44, 126.02, 126.83, 127.78, 128.62, 129.29, 132.23, 143.92, 149.34, 158.25, 160.71

### Example 112a: 1-(4-chlorolbenzyl)-6-chloro-2-(5-methyl-2-thienyl)benzimidazole

¹H-NMR (CDCl₃, 400 MHz) δ(ppm)

2.50 (3H, s, 5′-CH₃), 5.48 (2H, s-CH₂), 6.71 (1H, d, J=2.8H-4′), 7.01 (2H, d, J=8.8, H-2″, 6″), 7.06 (1H, d, J=4, H-5), 7.14 (1H, d, J=8, H-7), 7.29 (1H, s, H-3′), 7.31˜7.33 (2H, m, H-3″, 5″) 7.69 (1H, d, J=8.4, H-4)

¹³C-NMR (CDCl₃, 100 MHz) δ(ppm)

15.46, 47.84, 109.85, 120.74, 126.50, 127.28, 128.42, 128.92, 129.33, 129.57, 134.07, 134.17, 136.82, 141.81, 144.51, 149.16

### Example 112b: 1-(4-chlorolbenzyl)-5-chloro-2-(5-methyl-2-thienyl)benzimidazole

¹H-NMR (CDCl₃, 400 MHz) δ(ppm)

2.50 (3H, s, 5′-CH₃), 5.50 (2H, s-CH₂), 6.72 (1H, d, J=2.4, H-4′), 6.99 (2H, d, J=8, H-2″, 6″), 7.04˜7.08 (2H, m, H-5, 7), 7.15˜7.17 (1H, m, H-3′), 7.29 (2H, d, J=8, H-3″, 5″), 7.76 (1H, s, H-4)

¹³C-NMR (CDCl₃, 100 MHz) δ(ppm)

15.31, 47.73, 110.40, 119.46, 123.49, 126.37, 127.15, 128.50, 128.56, 129.37, 133.88, 134.09, 134.59, 143.16, 143.72, 144.53, 149.31

### Example 113a: 1-(4-fluorolbenzyl)-6-chloro-2-(5-methyl-2-thienyl)benzimidazole

¹H-NMR (CDCl₃, 400 MHz) δ(ppm)

2.53 (3H, s, 5′-CH₃), 5.52 (2H, s-CH₂), 6.75 (1H, s, H-4′), 7.06 (2H, d, J=8, H-2″, 6″), 7.15 (2H, d, J=3.2, H-3″, 5″), 7.19 (1H, s, H-5), 7.25 (1H, s, H-7), 7.27 (1H, s, H-3′) 7.23 (1H, d, J=8, H-4)

¹³C-NMR (CDCl₃, 100 MHz) δ(ppm)

15.21, 47.57, 109.71, 116.00, 116.22, 120.24, 123.62, 127.33, 127.40, 128.27, 128.50, 128.74, 130.94, 136.42, 141.05, 144.47, 148.73, 160.95, 163.42

### Example 113b: 1-(4-fluorolbenzyl)-6-chloro-2-(5-methyl-2-thienyl)benzimidazole

¹H-NMR (CDCl₃, 400 MHz) δ(ppm) 2.55 (3H, s, 5′-CH₃), 5.59 (2H, s-CH₂), 6.80 (1H, s, H-4′), 7.05˜7.07 (2H, m, H-2″, 6″), 7.08˜7.13 (2H, m, H-2″, 6″), 7.08˜7.13 (2H, m, H-3″, 5″), 7.14 (1H, d, J=8, H-5), 7.33 (1H, s, H-3′) 7.85 (1H, s, H-4)

¹³C-NMR (CDCl₃, 100 MHz) δ(ppm)

15.55, 48.10, 110.93, 116.32, 116.54, 119.04, 124.14, 126.86, 127.72 (d), 129.36, 130.10, 131.02, 134.26, 148.89, 161.31, 163.77

To practice the method of this invention, the above-described pharmaceutical composition can be administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation. An aryl substituted sulfonamide compound-containing composition can also be administered in the form of suppositories for rectal administration.

The carrier in the pharmaceutical composition must be “acceptable” in the sense of being compatible with the active ingredient of the formulation (and preferably, capable of stabilizing it) and not deleterious to the subject to be treated. One or more solubilizing agents (e.g., cyclodextrins) which form more soluble complexes with the active aryl substituted sulfonamide compounds can be utilized as pharmaceutical carriers for delivery of the active compounds. Examples of other carriers include colloidal silicon dioxide, magnesium stearate, sodium lauryl sulfate, and D&C Yellow #10.

Suitable in vitro assays can be used to preliminarily evaluate the efficacy of the aryl substituted sulfonamide compounds in anticancer activities such as inhibiting growth of tumor cells. The compounds can further be examined for their efficacy in treating cancer. For example, a compound can be administered to an animal (e.g., a mouse model) having cancer and its therapeutic effects are then assessed. Based on the results, an appropriate dosage range and administration route can also be determined.

Without further elaboration, it is believed that the above description has adequately enabled the present invention. The following examples are, therefore, to be construed as merely illustrative, and not limitative of the remainder of the disclosure in any way whatsoever. All of the publications cited herein are hereby incorporated by reference in their entirety.

### Benzimidazole Compounds in the Inhibition of Platelet Aggregation and Thrombus Formation

### Reagents and Animals

Indomethacin, fluorescein sodium, PGE1, Fura 2-AM, Bovine serum albumin (BSA), prostaglandin E1 (PGE1), EDTA, dimethylsulfoxide (DMSO), aspirin, heparin, and thrombin were purchased from Sigma Chem. (St. Louis, Mo.). Anti-phosphotyrosine mAb (4G10) was from Upstate Biotechnology. (Lake Placid, N.Y., USA). Horseradish peroxidase (HRP)-conjugated anti-mouse Ab and enhanced chemiluminescence (ECL) detection system were purchased from Santa Cruz Biotechnology (Autogen Bioclear UK Ltd, Calne, Wilts, UK). Arachidonic acid and U46619 were purchased from Cayman Chemical (Ann Arbor, Mich., USA). Fluorescein-5-isothiocyanate (FITC) was from Molecular Probe (Eugene, Oreg.). Thromboxane B2 kit was purchased from Amersham (Buckinghamshire, HP, UK). Male ICR mice were obtained from the Lab Animal Center, College of Medicine, National Taiwan University.

### Preparation of Human Platelet Suspension and Platelet-Rich Plasma

Human platelet suspension (PS) was prepared according to the method described previously. Briefly, blood sample, freshly obtained from healthy volunteers who had taken no medications during the preceding 2 weeks, was treated with acid citrate/dextrose (ACD) in a volume ratio of 9:1. After centrifugation at 120 g, 25° C. for 9 min, platelet-rich plasma was transferred into another plastic tube and incubated with heparin (6.4 U/ml) as well as prostaglandin E1 (1 μM). Platelets were spun down by centrifugation at 500 g, 25° C. for 8 min and subsequently washed two times with Tyrode solution (NaH2PO4 0.4 mM; NaCl 136.9 mM; KCl 11.9 mM; NaHCO3 11.9 mM; CaCl2 2 mM; MgCl2 2.1 mM; glucose 11.1 mM; BSA 3.5 mg/ml; pH 7.35-7.4). The washed platelets were finally suspended in Tyrode's solution and the platelet count was adjusted to 3.75×10⁸ platelets/ml. For platelet-rich plasma (PRP) preparation, whole blood was anticoagulated with 1/10 vol of sodium citrate (3.8%, w/v) and centrifuged at 150 g, 25° C. for 9 min.

### Measurement of Platelet Aggregation

Platelet aggregation was measured turbidimetrically with a Lumi-aggregometer (Payton Scientific) at 37° C. under stirring condition (900 rpm). Platelet suspensions were prewarmed to 37° C. for 2 min in a silicone-treated glass cuvette. Nstpbp5185 was added 3 min before the addition of platelet-aggregation inducers. The extent of platelet aggregation was determined by recording the increase of light transmission relative to that of control (maximal aggregation, as 100%) for 6 min after the addition of inducer. The final volume and platelet count for measuring platelet aggregation were 500 μl and 3×10⁸/ml, respectively.

### Measurement of Platelet [Ca²⁺]i Mobilization by Fura 2-AM Fluorescence

After centrifugating platelet-rich plasma at 500 g, 25° C. for 8 min, isolated platelets were resuspended in Ca²⁺-free Tyrode solution. The platelet suspensions were protected from light and incubated with Fura 2-AM (5 μM) at 37° C. for 30 min. Human platelets were then prepared as described previously. The washed platelet count was adjusted to 3-4.5×10⁸ platelets/ml. Finally, the external Ca²⁺ concentration of the platelet suspensions was adjusted to 1 mM. The rise in [Ca²⁺]i was measured using a fluorescence spectrophotometer (CAF 110, Jasco, Japan) at excitation wavelengths of 340 and 380 nm, and an emission wavelength of 500 nm. [Ca²⁺]i was calculated from the fluorescence, using 224 nM as the Ca²⁺-Fura 2 dissociation constant.

### Flow Cytometric Analysis of P-Selectin Expression

The washed platelet suspension (3.75×108/ml) containing tirofiban (100 ng/ml) was preincubated with nstpbp5185 for 3 min at 37° C. and activated with collagen or thrombin for 10 min at 37° C. Then the sample was further incubated with FITC-labeled anti-CD62P in the dark at room temperature. Prior to analysis, the volume was adjusted to 1 mL/tube with Tyrode's solution. The suspensions were immediately assayed by fluorescence activated cell sorter (Becton-Dickinson, FACScan System, San Joe, Calif.) using excitation and emission wavelength of 488 and 525 nm, respectively. Data were collected from 10000 platelets per experimental group. The level of P-selectin expression was expressed as mean fluorescence intensity.

### Measurement of Thromboxane B2 Formation

Human platelet suspension (3.75×108/ml) was preincubated in the presence or absence of nstpbp5185 or aspirin for 3 min before the addition of collagen or AA. Six minutes after the addition of agonist, 50 g M indomethacin and 2 mM EDTA were added to the reaction suspension. After centrifugation in an Eppendorf centrifuge at 14,000 g for 2 min, the thromboxane B2 (TXB2), the stable metabolite of TXA2, levels of the supernatant were measured using a competitive enzyme-immuno assay (EIA) kit according to the instructions of the manufacturer.

### Thromboxane A2 Synthase, COX and Other Kinases or Enzymatic Activity Assay

These kinases or enzymatic assays were performed by MDS according to the standardized methods

### Effect of Nstbp05185 on 3H-SQ-29548 Binding to TP Receptor of Recombinant HEK-293 Cells

Freshly prepared samples of this suspension were incubated with [³H]SQ-29548 (5 nM final concentration), for 60 min at 25° C. The displacement was initiated by addition of nstpbp5185 dissolved in the same buffer. After incubation (30 min, 25° C.), ice-cold Tris-HCl buffer (10 mM, pH 7.4) was added, the sample was rapidly filtered through a glass fiber filter, and the tube rinsed twice with ice-cold buffer. The filters were then placed in plastic scintillation vials containing an emulsion-type scintillation mixture, and the radioactivity was counted.

### Fluorescein Sodium-Induced Platelet Thrombus Formation in Mesenteric Venules of Mice

A modification of the method was used. Male ICR mice (12-14 g) were anesthetized with sodium pentobarbital (50 mg/kg, i.p.). The fluorescein sodium (12.5 μg/g) were injected i.v. through a lateral tail vein of the mouse. Five min later, various doses of nstpbp5185 (2 and 4 μg/g), aspirin (150 and 250 μg/g), or DMSO (control) were administered by i.v. through other lateral tail vein. The injected volume of the nstpbp5185, aspirin, or DMSO (control) was smaller than 10 μl. A segment of the small intestine with its mesentery attached was exteriorized through a midline incision on the abdominal wall and placed onto a transparent plastic plate for microscopic observation. Frequent rinsing of the mesentery with warm isotonic saline kept at 37° C. was performed to prevent the mesentery from drying out. Microvessels in the mesentery were observed under transillumination from a halogen lamp. Venules with diameters of 30-40 μm were selected to be irradiated to produce a microthrombus. In the epi-illumination system, light from a 100-W mercury lamp was filtered through a filter (B-2A, Nikon, Tokyo, Japan) with a dichromic mirror (DM 510, Nikon). The filtered light eliminated wavelengths below 520 nm irradiated a microvessel through an objective lens (20×). The area of irradiation was about 100 μm in diameter on the focal plane. Ten minutes after administration of the dye, the irradiation by filtered light and a timer were simultaneously started, and thrombus formation was observed on a TV-monitor. Occlusion time was recorded as the time interval for inducing thrombus formation leading to cessation of blood flow.

### Ex Vivo Antithrombotic Effect of Nstpbp5185 in Mice

Mice were anesthetized with sodium pentobarbital (50 mg/kg, i.p.). Mice were intravenously injected with saline or nstpbp5185 (at 4, and 10 μg/g), and then blood was collected at 15, 30 and 60 min after injection. PRP was prepared immediately by centrifugation at 200×g for 5 min and platelet aggregation assays were performed using collagen (10 μg/ml) as the aggregation inducer.

### Effect of Nstpbp5185 on Thromboembolism Caused by Collagen/Epinephrine

Male mice (ICR, 25±3 g) received collagen (0.6 μg/g) plus epinephrine (0.2 μg/g) by tail vein injection. While the respiration is very weak and the heart was still beating, 0.5 mL of Evans blue solution (1% in saline; Sigma) was injected into the heart. Lungs were excised, photographed and fixed with paraldehyde, embedded into paraffin, and sectioned. Histological analysis was performed on hematoxylin and eosin-stained (H&E) sections of lung from each mouse. Nstpbp5185 (10 μg/g) or DMSO vehicle was intravenously given prior to collagen/epinephrine. Mortality after challenge was calculated as percentage of death after injection of collagen/epinephrine, the end time will be 25 hr.

### Comparative Study on the Gastric Damage Between Aspirin and Nstpbp5185

Rats (Wistar) were fasted overnight then given aspirin at dose of 150 mg/kg or nstpbp5185 at 40 mg/kg. Both drugs were suspended in 1% carboxymethylcellulose. Three hours later, the rats were sacrificed, the stomach excised, and the extent of hemorrhagic erosions scored by an observer unaware of treatment the rats received. Each lesion larger than 2 mm was counted for comparison. The stomach was fixed in formalin (pH, 7.4) and photographed.

### Tail Bleeding Time

Male ICR mice were injected i.v. through a lateral tail vein of the mouse with DMSO, nstpbp5185 (2, 4 and 40 μg/g), or aspirin (150 and 250 μg/g). After 5 min, the mouse was placed in a tube holder with its tail protruding and 2 mm segment from the distal tail was severed. The amputated tail was immediately immersed in isotonic saline at 37° C. Bleeding time was recorded for a maximum of 1800 s and the end point was the arrest of bleeding.

### Atheroscelrosis Experimental Animal Model

Apo E-deficient mice (backcrossed for at least 10 generations to the C57BL/6J background) were obtained from Jackson Laboratories (Bar Harbor, Me.) at 8 weeks of age. All protocols conform to the Guide for the Care and Use of Laboratory Animals published by the US National Institutes of Health (NIH Publication No. 85-23, revised 1996) and was approved by the National Taiwan University Animal Care and Use Committee. Only male mice were used in the studies. Starting at 9 weeks of age, mice were fed a high-cholesterol diet (normal chow supplemented with 0.15% cholesterol and 4.6% fat), divided into four groups, and randomized to receive placebo, aspirin (30 mg/kg/day) or nstpbp05185 (20, 40 mg/kg/day) for 12 weeks. The doses used in our studies were based on previous in vivo experiments as an effective antithrombotic agent (data not shown). Urine was collected overnight in metabolic cages. The dosage of drug was calculated on the basis of the average consumption of water (5 mL/day) and the body weight, adjusted weekly. Mice were treated with vehicle, aspirin or nstpbp5185 from 9 weeks of age to 21 weeks of age. After treatment, mice were sacrificed using sodium pentobarbital and tissues were stored at −80° C. or in 24% formalin until assessment.

### Balloon-Injury Rat Carotid Artery Model

We used the established rat carotid artery (CA) model of balloon angioplasty to examine the in vivo arterial response to injury²⁰. Briefly, male Wistar rats weighing 350-400 g were anesthetized with intraperitoneal pentobarbital (50 mg/kg), and the left CA was exposed. A Fogarty 2F embolectomy balloon catheter was inserted into the left external carotid artery through arteriotomy incision and advanced to the aortic arch. The balloon was inflated and withdrawn three times with rotation at the same pressure. Followed by removal of the catheter, the external carotid artery was ligated and the wound closed. Animals were given standard rat chow and tap water, and i.p. administered 1 and 2 mg/kg or vehicle daily for 2 weeks, at which time they were sacrificed, and the sections from both the right and left (collateral control group) CA were harvested to fix with 4% paraformaldehyde in PBS overnight for study. All procedures involving animal experiments were approved by the Institutional Animal Care and Use Committee of Collagen of Medicine, National Taiwan University.

### Sensitization and Airway Challenge

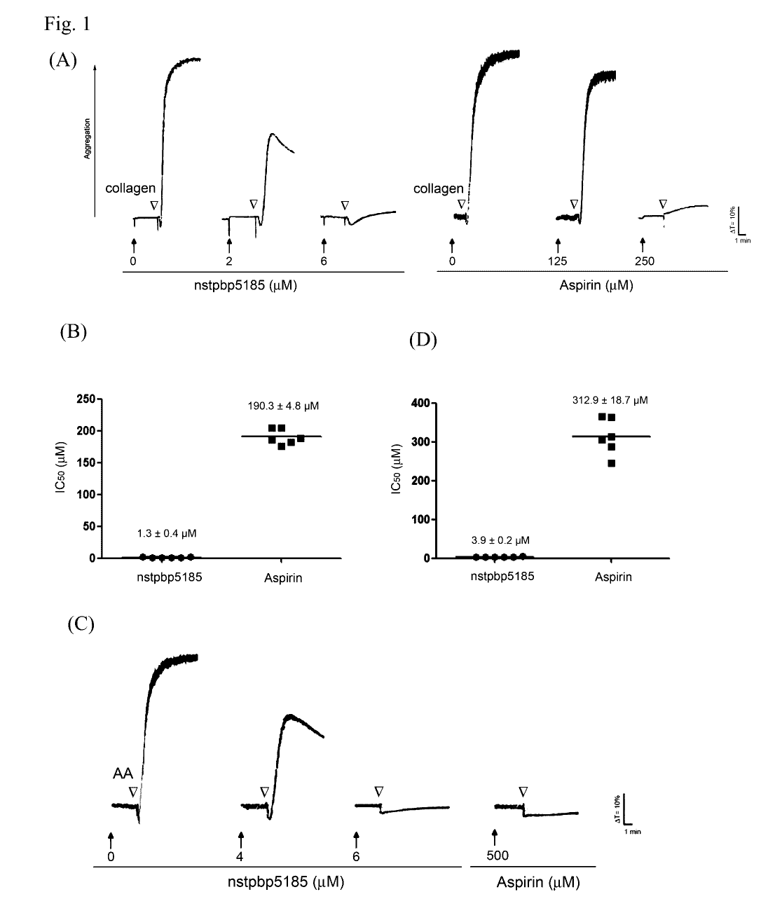

Mice were divided into groups receiving the following treatments: (1) sham sensitization plus challenge with phosphate buffered saline (PBS) (n=5), (2) sensitization plus challenge with OVA (Sigma A5503; Sigma, St. Louis, Mo.) (n=10), and (3 and 4) sensitization with OVA (i.p.) plus challenge with and nstpbp5185 (10 or 20 mg/kg) (n=6-7). Briefly, as shown in FIG. 1, mice were sensitized with an intraperitoneal injection of 50 μg OVA emulsified in 2 mg aluminum hydroxide in 200 μL PBS buffer (pH 7.4) on day 0, and 50 μg OVA emulsified in 2 mg aluminum hydroxide in 200 μL PBS buffer on day 14 and 28. Mice were challenged through the airway with 100 μg OVA in 40 μL PBS buffer via intranasal administration (i.n.) days 36, 37 and 38 and AHR induced by methacholine (MCh) were measured at day 39. Animals were sacrificed 24 h after the AHR measurement. The mice were placed in the main chamber of a whole body plethysmograph (Buxco Electronics, Inc., Sharon, Conn., USA) and challenged with aerosolized 0.9% normal saline accompanied by increasing doses of methacholine (6.25-50 mg/ml). Each nebulization lasted for 3 min, and after each nebulization, recordings were taken and averaged for the 3 min. The Penh values were determined, and the data were expressed as Penh values.

### Measurement of Total and OVA-Specific IgE in Serum

Serum were collected and stored at −70° C. after centrifugation (3000 rpm, 10 min). The amount of OVA-specific IgE was determined by ELISA. Briefly, 96-well plates were coated with OVA at 10 μg/well. After the plates were blocked, 100 μl/well of diluted sera was added, and the plates were incubated at room temperature for 2 h or at 4° C. overnight. After incubation, the plates were washed five times with PBS with Tween 20 buffer, the secondary antibody (biotinylated rat anti-mouse IgE; AbD Serotec, Kidlington, UK) was added and plates were incubated for 1 h at room temperature. After plates were washed, avidin-horseradish peroxidase (Pierce Chemical, Rockford, Ill., USA) was added, and samples were incubated at room temperature for 30 min. The avidin-horseradish peroxidase was removed by washing with PBS with Tween 20 buffer, and the bound enzyme substrate was detected by adding tetramethylbenzidine reagent (KPL, Gaithersburg, Md., USA). After incubation at room temperature for a short time, the reaction was stopped by adding 50 μl/well of 2 N H₂SO₄. Optical density was measured at 450 nm (550 nm was used as a reference filter) in a microplate autoreader (Anthos Reader 2010; Anthos Labtec Instruments GmbH, Salzburg, Austria).

### Statistical Analysis

The experimental results are expressed as the means±S.E.M. Data were analyzed by the unpaired or paired Students' t-test between two groups, and P value less than 0.05 was considered significant.

### Results

Table 1 shows the preliminary inhibitory effect of synthetic compounds (3-10) on platelet aggregation in human platelet suspension induced by collagen (10 μg/ml) and ADP.

| | | % inhibition | % inhibition |
|---|---|---|---|
| | structure | collagen | ADF |
| 3 | 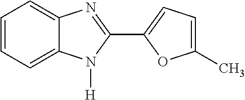 | 57.6 (n = 3) | 6.8 |
| 4 | 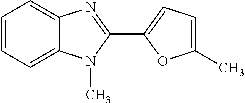 [SMILES: Cc1ccc(-c2nc3ccccc3n2C)o1] | 20.2 (n = 2) | 28.0 (n = 2) |
| 5 (nstpbp 5185) | 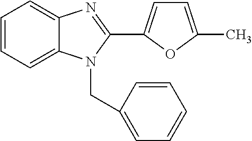 [SMILES: Cc1ccc(-c2nc3ccccc3n2Cc2ccccc2)o1] | 71.2 (n = 2) | 10.9 |
| 6 | 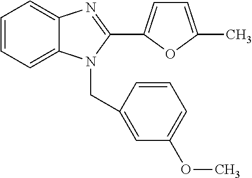 [SMILES: COc1cccc(Cn2c(-c3ccc(C)o3)nc3ccccc32)c1] | 19.1 | 41.4 (n = 2) |
| 7 | 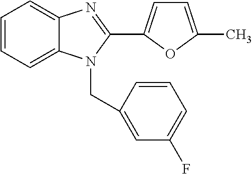 [SMILES: Cc1ccc(-c2nc3ccccc3n2Cc2cccc(F)c2)o1] | 49.9 (n = 3) | 26.1 (n = 2) |
| 8 | 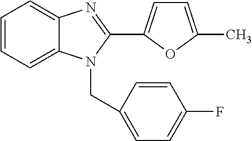 [SMILES: Cc1ccc(-c2nc3ccccc3n2Cc2ccc(F)cc2)o1] | 45.4 (n = 3) | 37.3 (n = 3) |
| 9 | 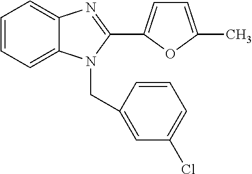 [SMILES: Cc1ccc(-c2nc3ccccc3n2Cc2cccc(Cl)c2)o1] | 1.1 | 17.4 (n = 3) |
| 10 | 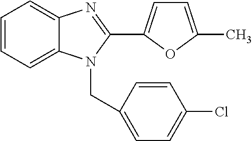 [SMILES: Cc1ccc(-c2nc3ccccc3n2Cc2ccc(Cl)cc2)o1] | 3.0 (n = 3) | 46.1 (n = 3) |

### Effect of Nstpbp5185 on Platelet Aggregation in Human Platelet Suspension and Platelet-Rich Plasma

To investigate whether exhibits inhibitory actions on platelet aggregation, human platelet suspension (PS) was employed. As shown in FIG. 1A, nstpbp5185 caused a concentration-dependent inhibition on platelet aggregation induced by collagen (10 μg ml⁻¹) in human PS. The IC₅₀ values of nstpbp5185 or aspirin in suppressing collagen-induced platelet aggregation in human PS were approximately 1.3±0.4 μM and 190.3±4.8 μM, respectively (n=6) (FIG. 1B). However, nstpbp5185 only had little effects on platelet aggregation caused by thrombin or ADP (data not shown). Furthermore, the IC₅₀ values of nstpbp5185 and aspirin in suppressing collagen-induced platelet aggregation in human PRP were 64.2±2.5 μM and 5.1±0.7 mM, respectively (data not shown). Hence, nstpbp5185 exhibits a greater potency in inhibiting platelet aggregation both in human PS or PRP than aspirin does. On the other hand, nstpbp5185 also concentration-dependently blocked the platelet aggregation caused by arachidonic acid (AA) (FIG. 1C). As shown in FIG. 1D, the IC₅₀ values of nstpbp5185 and aspirin in suppressing AA-induced platelet aggregation were 3.9±0.2 μM and 312.9±18.7 μM, respectively (n=6).

### Effects of Nstpbp5185 on Collagen-Stimulated [Ca²⁺]i Mobilization and P-Selectin Expression

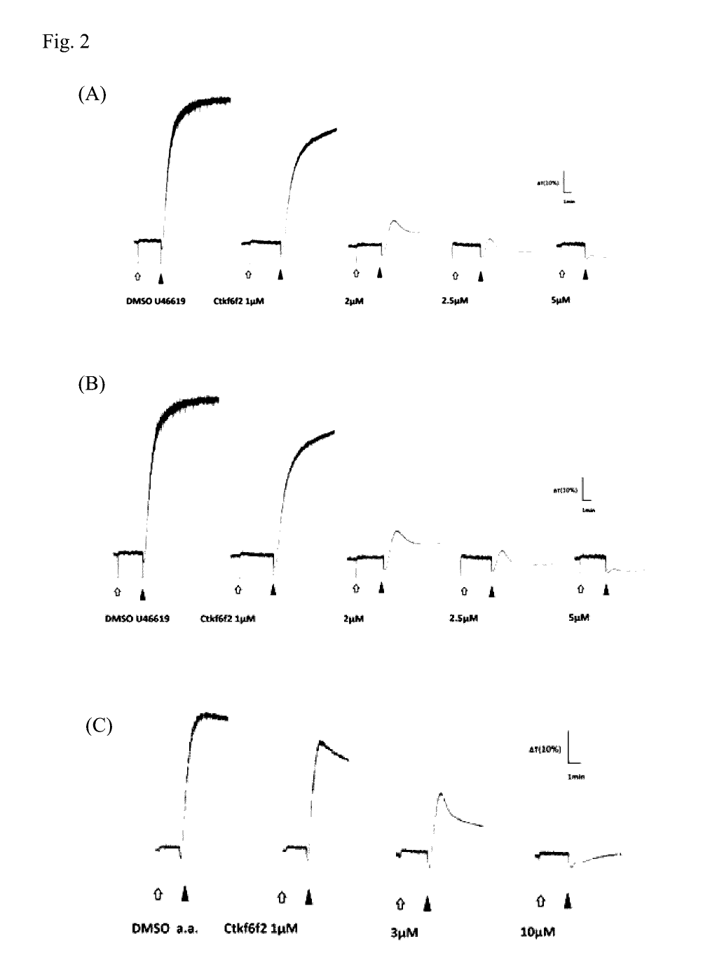

Based on the fact that the increase in [Ca²⁺]i plays important role in amplifying pathways involved in the stimulation of platelets, the effects of nstpbp5185 on aggregation and [Ca²⁺]i elevation in platelets stimulated with agonists were examined. The representative traces of two different agonists induced [Ca²⁺]i mobilization were shown in FIG. 2A. Collagen-evoked increase of [Ca²⁺]i was markedly inhibited by nstpbp5185 in a concentration-dependent manner. In contrast, nstpbp5185 has no effect on thrombin-evoked increase of [Ca²⁺]i even at high concentration of 60 μM (FIG. 2B). In addition to [Ca²⁺]i mobilization, platelet activation after stimulation is accompanied by degranulation. Measurement of the α-granule marker, P-selectin, was carried out. Following activation, P-selectin is rapidly translocated to the cell surface, its expression on platelets has been shown to be elevated in disorders associated with arterial thrombosis (29). In accord with its inhibitory effects on [Ca²⁺]i mobilization of collagen-stimulated platelet, the level of P-selectin expression was concentration-dependently reduced to baseline by nstpbp5185 (FIG. 2C). Furthermore, nstpbp5185 also significantly reduced the U46619-induced P-selectin expression (FIG. 2D).

### Effect of Ctkf6f2 on Platelet Aggregation

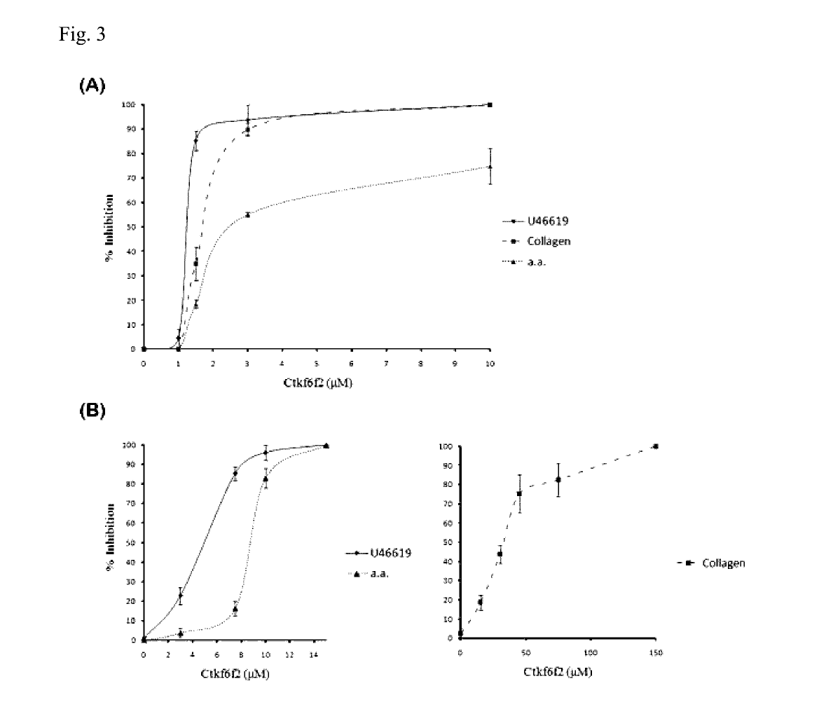

To begin with, potency of aggregation inhibition and estimated the IC50 of Ctkf6f2 on platelet aggregation caused by different agonists was investigated. As shown in FIG. 3, Ctkf6f2 inhibited U46619 (1 μM), collagen (10 μg/ml) and arachidonic acid (200 μM)—induced aggregation in a concentration-dependent manner in washed human platelets. The IC50 of Ctkf6f2 on U46619, collagen and arachidonic acid-induced aggregation are 1.17±0.23 μM, 3.4±0.62 μM and 3.2±0.99 μM, respectively. The IC50 value on U46619-induced aggregation is much lower than those on collagen and arachidonic acid-induced ones, suggesting Ctkf6f2 may target to thromboxane receptor, in coincidence with the property of compound 5185. In addition, the effect of Ctkf6f2 in platelet-rich plasma was also investigated. Ctkf6f2 increasingly inhibited platelet aggregation with the increase of concentration under various agonists. The values of IC50 induced by U46619 (1 μM), collagen (5 μg/ml) and arachidonic acid (200 μM) were 3.27±1.01 μM, 31.33±7.68 μM and 8.87±2.52 μM, respectively. The inhibition on thromboxane receptor shows distinctly in platelet-rich plasma which is closer to the normal blood circumstances. In the meanwhile, the value of IC50 by arachidonic acid hints that Ctkf6f2 may also inhibit the production of thromboxane A₂. Furthermore, concentration-dependent inhibition curve in platelet suspension and platelet-rich plasma could be observed as well from FIG. 3. Moreover, LDH assay in washed human platelets with various Ctkf6f2 concentration used in the following experiments was performed. Ctkf6f2 at 10 μM shows little cytotoxicity to platelets. This suggests that Ctkf6f2 does not affect platelet viability at the concentration used in this experiment.

TABLE 2
IC50 value of U46619-induced aggregation tested on
other synthetic compounds

| | P.S(U46619) |
|---|---|
| No. | IC50 (μM) |
| 6 | 2.45 |
| 9 | 4.63 |
| 13 | 13.09 |
| 22 | 4.29 |
| 23 | 3.01 |
| 24 | 14.6 |
| 25 | 44.48 |
| 25a | 1.45 |
| 25b | 3.3 |
| 27a | 19.25 |
| 27b | 26.9 |
| 29 | 33.67 |
| 30 | 52.075 |
| 30a | 6.79 |
| 31a | 22.725 |
| 31b | 12.655 |
| 32a | 4.26 |
| 32b | 3.09 |
| 34a | 34.46 |
| 34b | 32.23 |
| 37a | 1.7 |
| 37b | 1.17 |
| 38a | 4.14 |
| 38b | 2.7 |
| 39a | 41.34 |
| 39b | 12.56 |
| 40a | 14.59 |
| 40b | 5.39 |
| 41a | 2.61 |
| 41b | 2.38 |
| 42 | 37.265 |
| 42a | 3.55 |
| 42b | 4.43 |
| 43a | 36.54 |
| 43b | 126.74 |
| 44a | 9.74 |
| 44b | 3.1 |
| 45a | 46.3 |
| 45b | 7.52 |
| 46a | 3.13 |
| 46b | 9.59 |
| 47a | 5.55 |
| 47b | 4.42 |
| 48a | 6.7 |
| 48b | 3.11 |
| 49b | 29.63 |
| 50a | 37.96 |
| 50b | 27.45 |
| 51a | 53.39 |
| 51b | 62.82 |
| 52a | 29.88 |
| 52b | 5.22 |
| 53a | 6.01 |
| 53b | 3.2 |
| 54 | 5.13 |
| 54a | 6.98 |
| 55 | >50 |
| 56 | 27 |
| 57 | >50 |
| 58 | 9.56 |
| 59 | 3.74 |
| 60 | 42.91 |
| 61 | 26.9 |
| 62 | 8.88 |
| 63 | 2.68 |
| 64 | 1.74 |
| 65 | 8.32 |
| 66 | 40.27 |
| 67 | 6.52 |
| 68 | 5.69 |
| 69 | >50 |
| 69a | 17.22 |
| 73 | >50 |
| 74 | 19.22 |
| 76 | >50 |
| 77 | >50 |
| 78 | 25.78 |
| 79 | >50 |
| 80 | >50 |
| 81 | >50 |
| 83 | >50 |

### Effect of Ctkf6f2 on Collagen and U46619-Induced Intracellular Ca²⁺ Mobilization

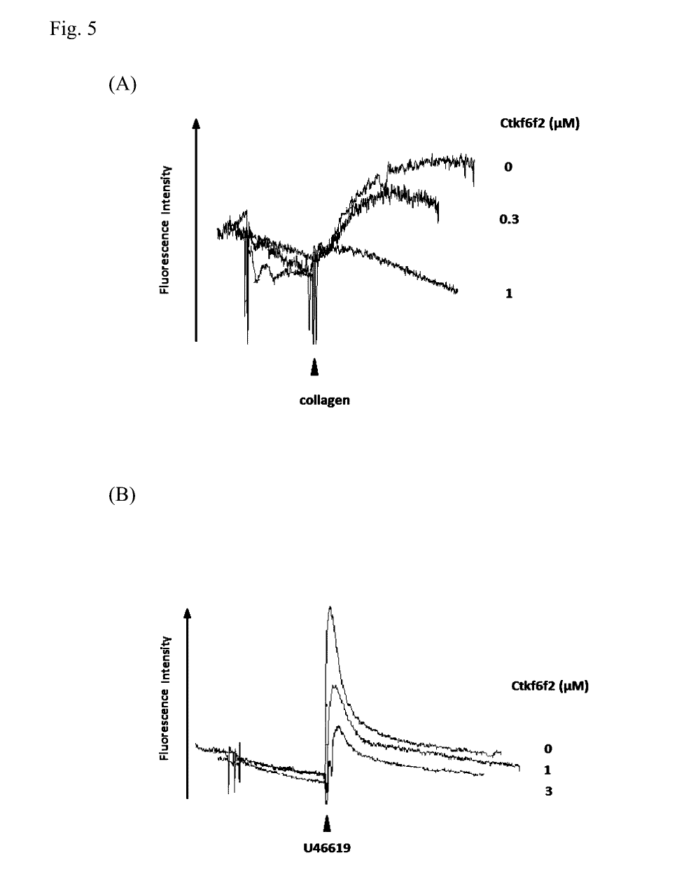

The increase of [Ca²⁺]i amplifies the signal pathway of platelet aggregation. The effect of Ctkf6f2 on [Ca²⁺]i after adding collagen and U46619 as agonist was examined. By measuring the fluorescence intensity, the calcium mobilization was estimated. It was found that Ctkf6f2 0.3 μM-1 μM concentration-dependently inhibited collagen-induced [Ca²⁺]i increase. Likewise, the calcium mobilization caused by U46619 was markedly inhibited by Ctkf6f2 in a concentration-dependent manner (1 μM-3 μM) (FIG. 5).

### Effect of Ctkf6f2 on Collagen and U46619-Induced Granule Secretion

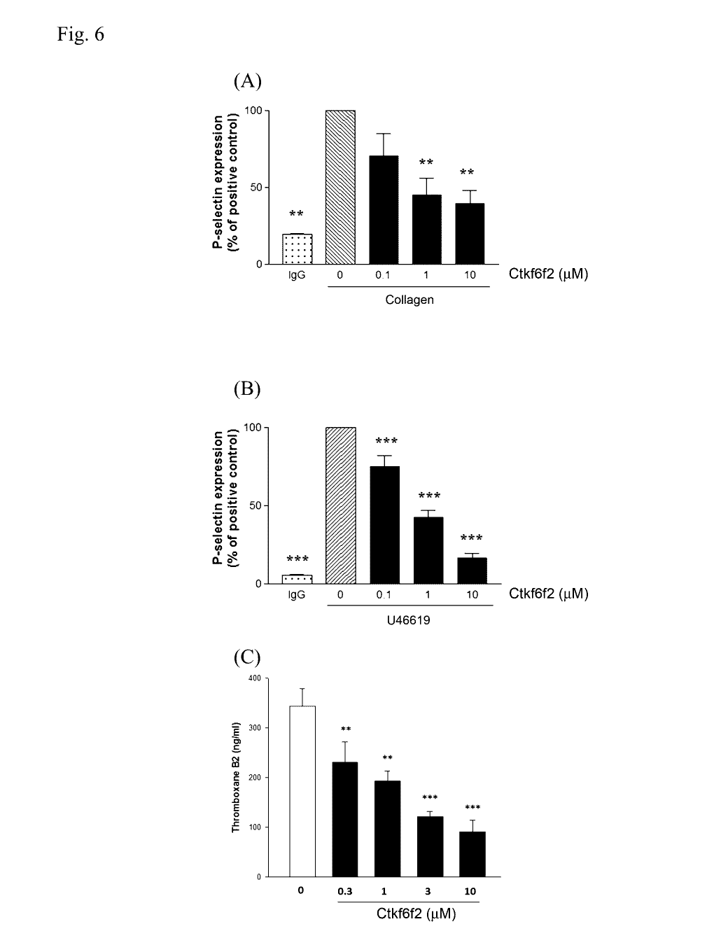

After the stimulation, platelet activation is accompanied by granule secretion. P-selectin is a critical adhesion molecule released from α-granule which rapidly translocates to the cell surface following the activation. The expression of P-selectin as α-granule marker was measured. As shown in FIG. 6, the level of P-selectin expression caused by collagen or U46619 was decreased by Ctkf6f2 in a concentration-dependent manner.

### Inhibitory Effects of Nstpbp5185 on Thromboxane A2 Formation of Human Platelets Caused by Collagen and Arachidonic Acid

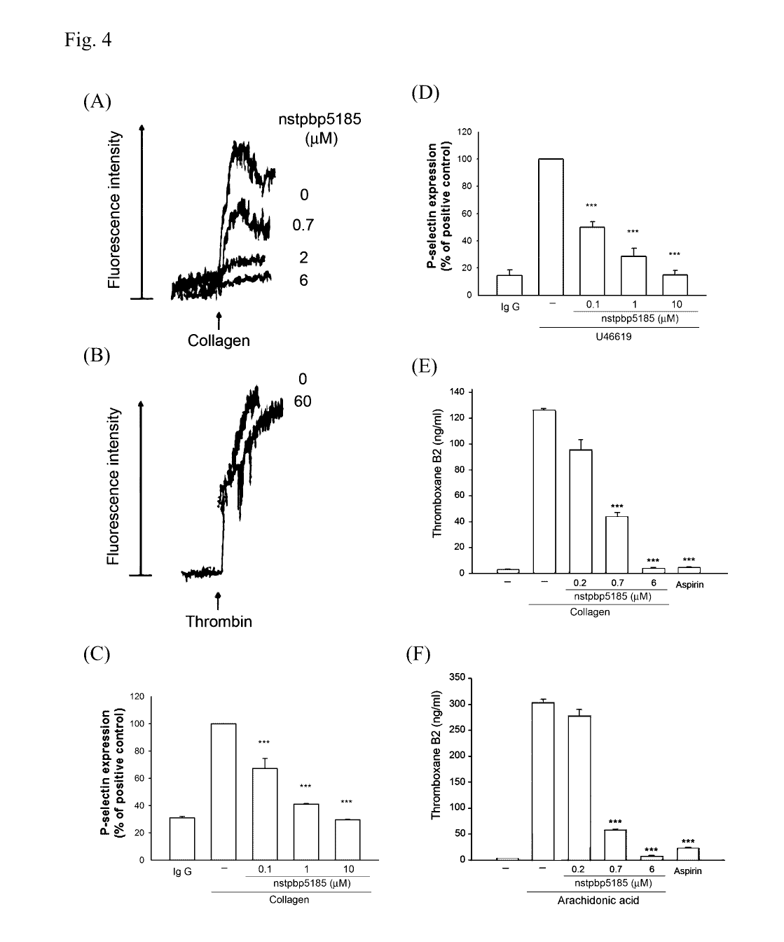

It is known that collagen depends on the release of secondary mediators in triggering aggregation (30, 31). Thus, the effect of nstpbp5185 on the production of the stable TXA₂ metabolite, thromboxane B₂ (TXB₂), was examined in washed platelet suspension. As shown in FIG. 4E, resting platelets produced relatively little TXB₂ compared with those of collagen-activated platelets. Aspirin (500 μM) inhibited TXB₂ formation by 96%. Nstpbp5185 concentration-dependently inhibited TXB₂ generation of collagen-stimulated platelets, in parallel with their inhibitory activities on platelet aggregation. In addition, it also concentration-dependently inhibited TXB₂ generation of AA-stimulated platelets (FIG. 4F). Thus, the results implied that antiplatelet effect of nstpbp5185 may be due, at least partly, to the inhibition of TXA₂ formation.

### Effect of Ctkf6f2 on TXB₂ Formation of Human Platelets by Collagen

Collagen depends on the release of secondary mediators such as TXB₂ in triggering aggregation. Therefore, the effect of Ctkf6f2 on the production of the stable TXA₂ metabolite TXB₂, was measured in human washed platelets. Ctkf6f2 in 0.3, 1, 3, 10 μM inhibited 33.0%, 43.9%, 64.7%, 73.5% of TXB₂ compared to the control, respectively. Compound 5185 at 1 μM inhibited 52.6% of TXB₂ formation (FIG. 6C).

### Nstpbp5185 Competitively Inhibits U46619-Induced Platelet Aggregation Through TXA₂ Receptor Blockade

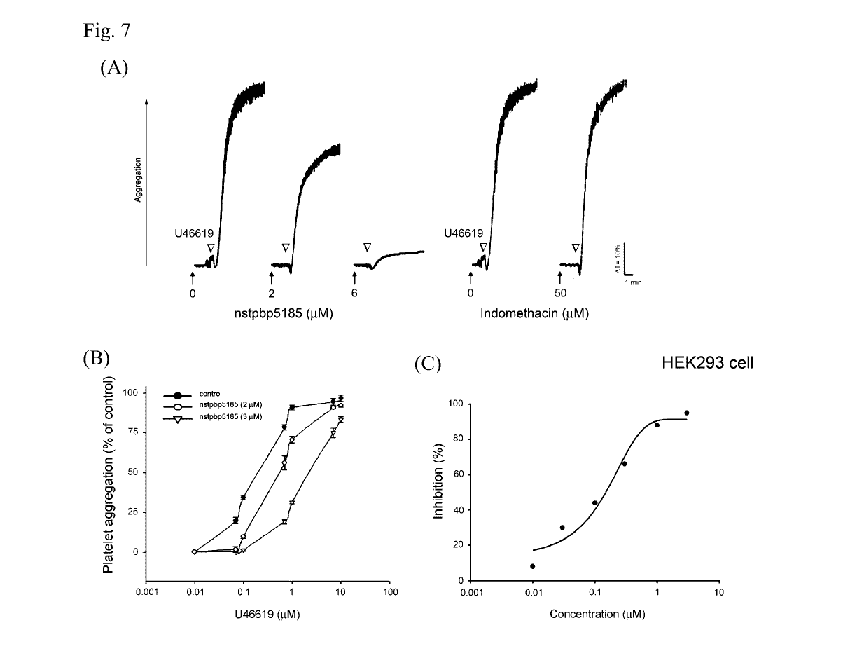

We next determined whether nstpbp5185 affects platelet aggregation induced by U46619, a stable TXA₂ mimic (a TP receptor agonist) in human PS. As shown in FIG. 7A, nstpbp5185 markedly suppressed U46619-induced platelet aggregation while indomethacin (a cyclooxygenase inhibitor) was without effects. The IC₅₀ value of nstpbp5185 in suppressing U46619-induced aggregation is approximately 2.5±0.1 μM (n=4). Moreover, aspirin at 500 μM had no effect on U46619-induced platelet aggregation (data not shown). To exclude any possible contribution of endogenous AA metabolites to platelet aggregation, the inhibitory effects of nstpbp5185 on U46619-induced platelet aggregation were further confirmed in the presence of indomethacin, a cyclooxygenase (COX) inhibitor. As shown in FIG. 7B, nstpbp5185 concentration-dependently blocked platelet aggregation elicited by U46619 under COX blockade with indomethacin. At concentrations of 2 and 3 μM, nstpbp5185 produced a right-shift of the concentration-response curve of U46619, suggesting a possible competitive antagonism on TXA₂ receptor (FIG. 7B). In parallel, a competitive binding assay was performed. As shown in FIG. 7C, the IC₅₀ value of nstpbp5185 in displacing ³H-SQ-29548 from TP receptor expressed on HEK-293 cells was estimated to be about 0.1 μM.

### Effect of Competitive Inhibition of Compound 5185 and Ctkf6f2 on U46619-Induced Platelet Aggregation

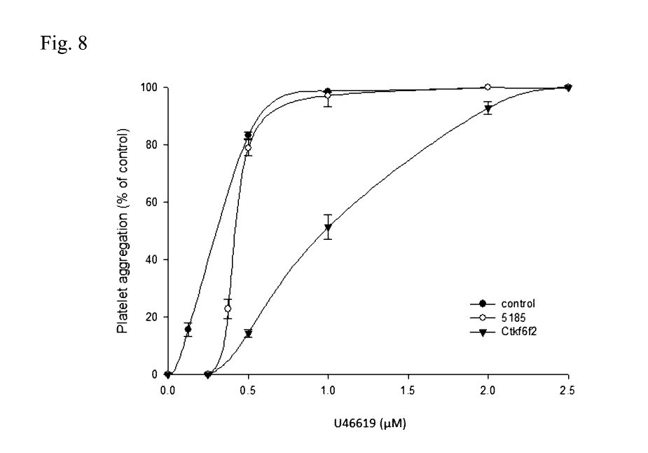

In order to prove that 5185 and Ctkf6f2 binds to the TP receptor, U46619, a mimetic compound of TXA₂ was used to challenge TP receptor. As shown in FIG. 8, under the same pre-treated concentration of Ctkf6f2, the aggregation level was rised with the increase of given U46619, suggesting that Ctkf6f2 and U46619 may compete the same receptor, in a similar way to compound 5185. Comparing the two compounds, when pretreated with 5185 at 1 μM, it needs 0.5 μM of U46619 to induce the full aggregation; however, when pretreated with Ctkf6f2, it needs around 2 μM of U46619 to achieve the aggregation. In brief, Ctkf6f2 shows a better affinity in competing TP receptor, consisting with a lower IC50 (1.17 μM) as compared to compound 5185 (2.45 μM). Ctkf6f2 produced a right-shift of the concentration-response curve of U46619 alone, suggesting a competitive antagonism of platelet TP receptors.

### Effect of Ctkf6f2 and Compound 5185 on In Vivo Thrombosis Mouse Model

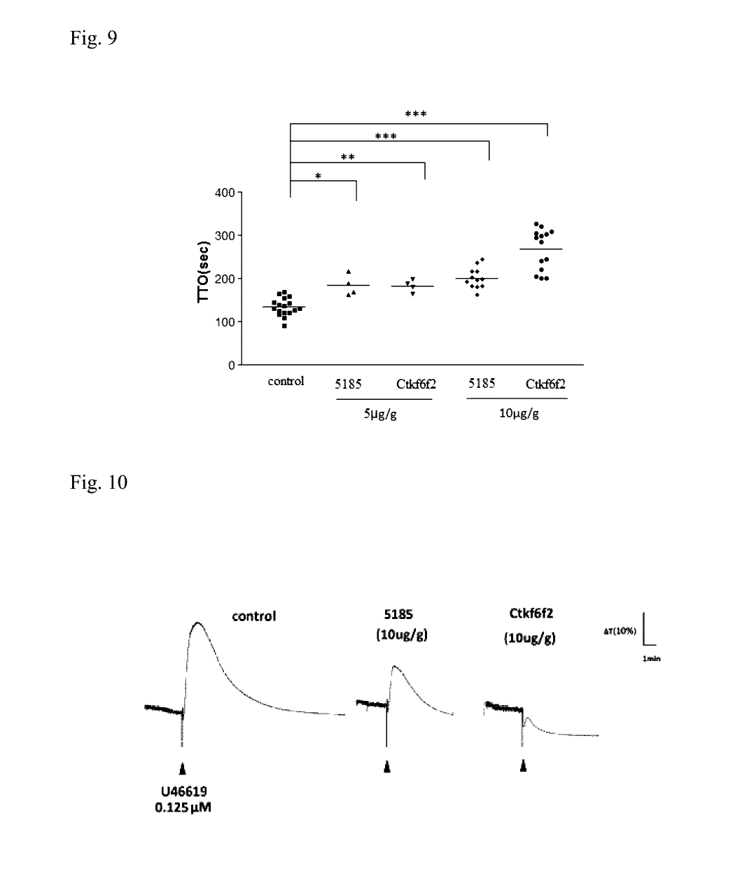

A mesenteric venous thrombosis model was used for estimating whether Ctkf6f2 exhibits antithrombotic activity in vivo. After giving 50 μg/g fluorescein sodium, the irradiation of mesenteric venules induced thrombus formation which is consist of activated platelets and fibrin clots. As shown in FIG. 9, administration of compound 5185 or Ctkf6f2 prolonged the time to occlusion (TTO) value. In the vehicle-treated group, the average TTO was approximately 143.71±4.99 sec (n=17). The group pre-treated with compound 5185 (10 μg/g), the average TTO value was 208.67±6.99 sec (n=12). And when the Ctkf6f2 (10 μg/g) was pre-given, the average TTO value was prolonged to 267.93±12.64 sec (n=14), which is significantly longer than the control group. These findings demonstrate that Ctkf6f2 has therapeutic potential in thrombosis as an antithrombotic agent.

### Effect of Ctkf6f2 and Compound 5185 on Ex Vivo Mouse Platelet Aggregation

Furthermore, the platelet aggregation was evaluated on ex vivo mouse model. The mouse was i.v. administered with compound 5185 (10 μg/g) or Ctkf6f2 (10 μg/g), compared to the vehicle-treated control. The blood was collected and evaluated for its response to U46619 (0.125 μM) on the aggregometer (FIG. 10).

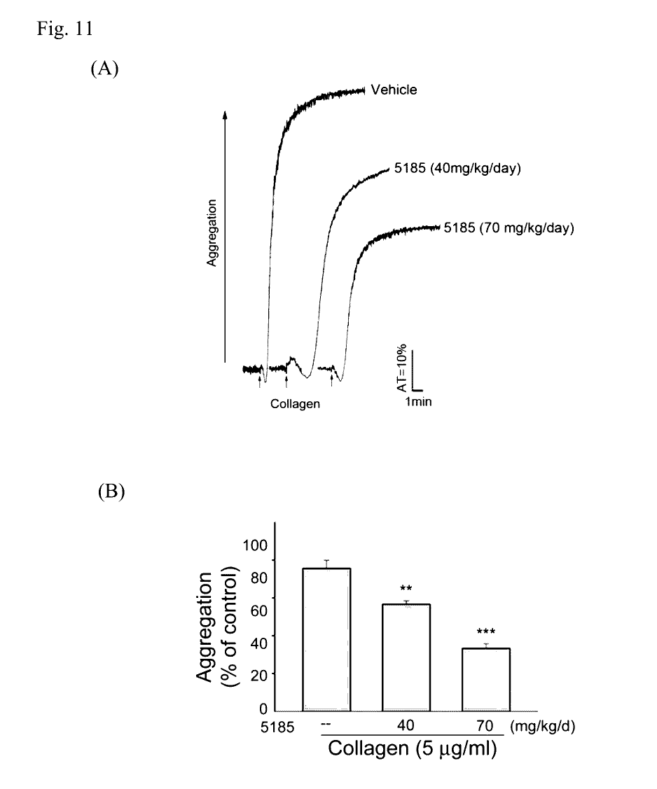

In human washed platelets, 1 μM of Ctkf6f2 could inhibit 50% of aggregation induced by U46619 (1 μM). However, 1 μM of nstpbp5185 could not effectively inhibit aggregation induced by U46619. The inhibition extent is distinct in human washed platelet. In mice i.v. injected with vehicle, 5185 (10 μg/g) or Ctkf6f2 (10 μg/g), which is the calculated equivalent concentration used in vitro, a superior inhibition of Ctkf6f2 was shown on platelet aggregation of PRP induced by U46619 (0.125 μM) as compared to that of nstpbp5185 in FIG. 11.

### Effect of Nstpbp5185 on Thrombus Formation in Microvessels of Fluorescein Sodium-Pretreated Mice

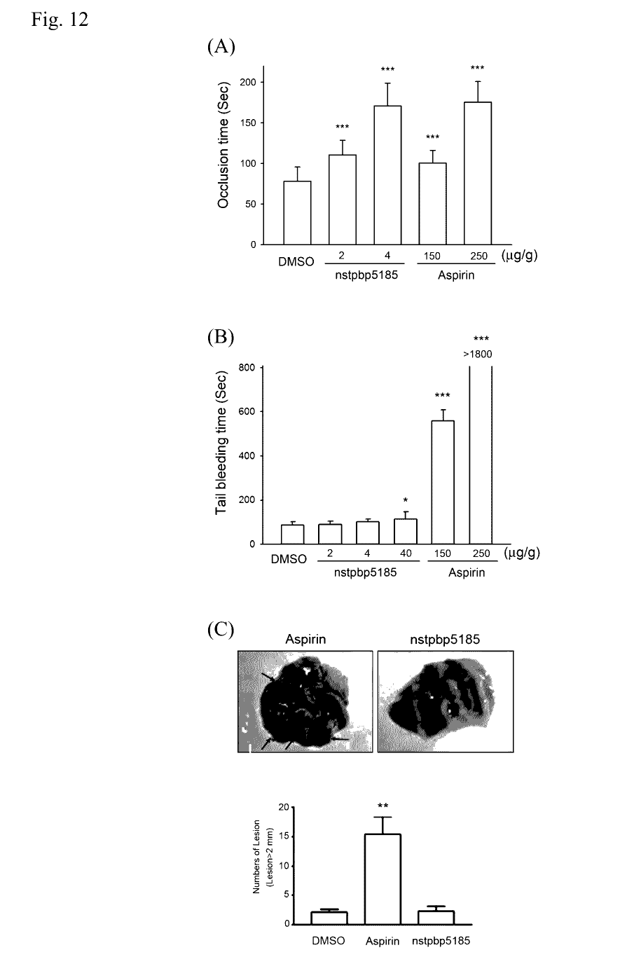

These promising in vitro profile of nstpbp5185 presented above led us to further examine whether nstpbp5185 has potential therapeutic potential in the treatment of thrombosis in vivo. Thus, its effect was evaluated with a mouse model of mesenteric venous thrombosis. From the data shown in FIG. 12A, after intravenous injection of nstpbp5185 significantly prolonged the occlusion time to 110.5±18.0 and 170.9±28.0 s, respectively (Control, 78.1±17.5 s). The occlusion time was also significantly prolonged within 70 min (157.5±12.5 s) after drug administration and returned to the control value within 90 min after nstpbp5185 administration (4 μg g⁻¹). On the other hand, low dose of aspirin did not significantly change the occlusion time until 250 μg g⁻¹ was administered. At a dose of 250 μg g⁻¹, aspirin increased the occlusion time to 175.5±25.3 s and the occlusion time was significantly prolonged within 40 min (155.0±7.1 s) and returned to the control value within 60 min (82.6±9.4 s) after aspirin administration. These findings demonstrate that in this model, nstpbp5185 was more potent than aspirin in exhibiting antithrombotic activity in vivo.

### Effect of Nstpbp5185 on the Tail Bleeding Time of Mice

By performing the tail bleeding model, the in vivo effect of drugs on hemostasis or the risk of hemorrhage was examined. A comparison of nstpbp5185 with aspirin on bleeding time was shown in FIG. 12B. The tail bleeding time of vehicle-treated mice was measured to be 88.3±13.5 s. Aspirin (150 μg g⁻¹)-treated mice resulted in a prolonged tail bleeding time 5 min after injection and at a dose of 250 μg g⁻¹, aspirin prolonged the bleeding time up to 1,800 s. Under the same condition, at 2 and 4 μg g⁻¹, nstpbp5185 showed no significant effect on bleeding time compared with the control. At a higher dose of 40 μg g⁻¹ (a 20-fold higher dose), nstpbp5185 has a slightly significant effect on the tail bleeding time. Therefore, nstpbp5185 seems to be safer in posing less bleeding risk in comparison to aspirin.

### Lack of Gastric Damage of Nstpbp5185 in Rats

As compared to the marked lesions caused by high dose of aspirin orally (150 μg g⁻¹), oral administration of compound nstpbp5185 at 40 μg g⁻¹ apparently did not cause significant lesion (>2 mm) (FIG. 12C), indicating that nstpbp5185 exhibits little irritating effect on gastric mucosa.

### Nstpbp5185 Prevents the Lung Thromboembolism and Fatality Caused by Collagen/Epinephrine Challenge in Mice

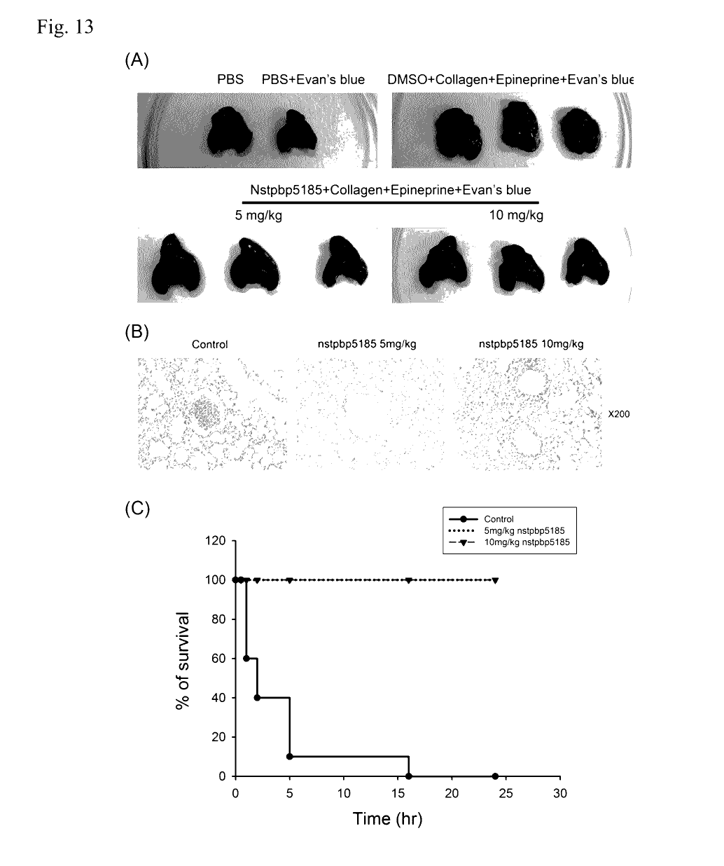

Pulmonary embolism is an important cause of morbidity and mortality in pulmonary vascular disease. The mortality rate caused by intravenous administration of collagen/epinephrine in vehicle control group was around 60% whereas nstpbp5185 (5 and 10 g g⁻¹, intravenous injection)-treated group completely protected mice from death within 1 hr (FIG. 13C). It was also found that the pulmonary circulation was obstructed to a greater extent in control mice as indicated by increased exclusion of Evans blue dye from the lungs compared to nstpbp5185-treated mice (FIG. 13A). Histological examination of lung sections showed numerous microemboli in the control mice whereas the nstpbp5185-treated mice showed few microemboli (FIG. 13B).

### Effect of Nstpbp5185 on Thrombus Formation in Carotid Artery FeCl₃-Induced Thrombosis Model

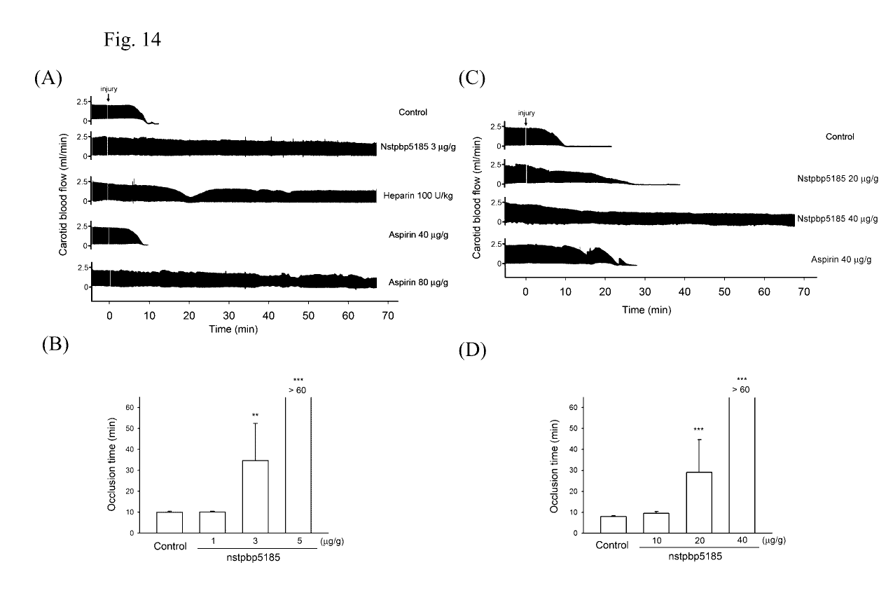

Mice were intravenously or orally administered with different doses of nstpbp5185. As shown in FIG. 14A, 10% FeCl₃ induced occlusion in about 10 min in vehicle-treated mice, nstpbp5185 (3-5 μg g⁻¹, intravenous administration) significantly blocked the FeCl₃-induced thrombus formation (FIG. 14B, ED₅₀≈2.6 μg g⁻¹). On the other hand, heparin (10 U kg⁻¹) or aspirin (40 μg g⁻¹) also induced occlusion in about 10 min, however heparin (100 U kg⁻¹) or aspirin (80 μg g⁻¹) at higher dose blocked the FeCl₃-induced thrombus formation (FIG. 14A). Furthermore, oral administration of nstpbp5185 (20-40 μg g⁻¹) significantly blocked the FeCl₃-induced thrombus formation (EC₅₀≈20.6 μg g⁻¹, FIGS. 14C and D). These data confirmed that nstpbp5185 is orally active and dose-dependently attenuates platelet aggregation and coagulation caused by endothelial/platelet activation in vivo.

### Effect of Ctkf6f2 on Pulmonary Histology and Pulmonary Thromboembolism

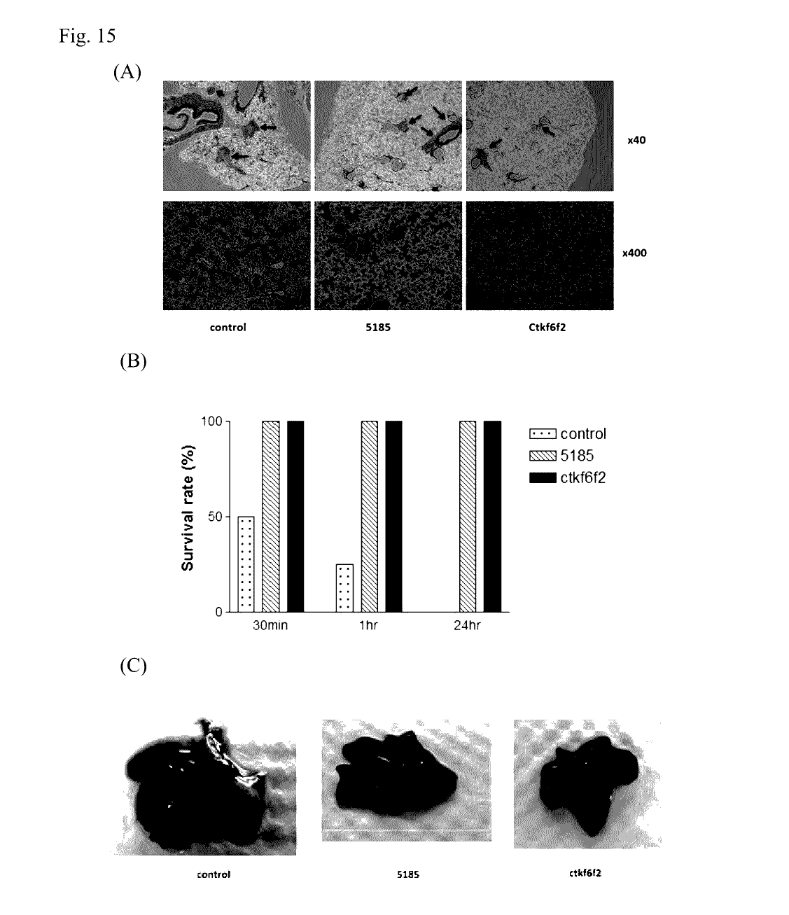

We used collagen plus epinephrine-induced pulmonary thromboembolism model to examine the histologic change of lung. There were numerous microemboli in section of DMSO given control group. Comparing the effect of two compounds, there are smaller emboli in administration of Ctkf6f2 (10 μg/g) group than the group in 5185 (10 μg/g). The beneficial effect of thrombi formation is shown as FIG. 15A under 10×4 and 10×40 magnification. Taken together, these results show that thrombogenesis is reduced after administrating of Ctkf6f2. Moreover, in a collagen plus epinephrine-induced pulmonary thromboembolism model, it was found that mice succumbed significantly faster while without pre-i.v. given Ctkf6f2 (10 μg/g) or 5185 (10 μg/g). The survival rate of control group after 30 min of induction is only 50%, and 25% for after 1 hr (FIG. 15B). However, for the group given Ctkf6f2 (10 μg/g) or 5185 (10 μg/g), the survival rate is up to 100% even after 1 day. Furthermore, the pulmonary circulation was obstructed to a lower extent in mice i.v. administered with Ctkf6f2 (10 μg/g) indicated by decreased exclusion of Evans blue dye from the lungs compared with the control or given 5185 (10 μg/g) (FIG. 15C). This suggests that Ctkf6f2 effectively inhibited pulmonary thromboembolism in mouse model.

Effects of Nstpbp5185 on Neointimal Formation after Arotid Artery Balloon Injury in Rats In Vivo

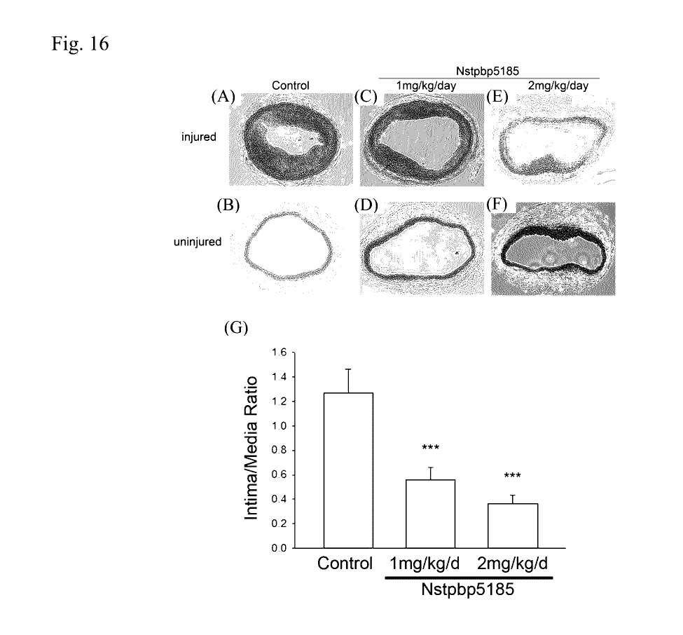

We further determined the inhibitory effect of nstpbp5185 on neointima formation after carotid artery injury. The animals were i.p. administered with nstpbp5185 at 1 and 2 mg/kg/day initially 3 days before injury and continuously throughout the 14-day postinjury period. Representative hematoxylin and eosin stained cross-sections from control (FIGS. 16A, B) and 1 mg/kg/day (FIG. 16C, D) or 2 mg/kg/day (FIG. 16E, F) nstpbp5185-treated animals are shown. Magnification for all photomicrographs is 100×. “N” indicates neointima, and “M” indicates media. (FIG. 16G) Data are quantified by the neointima/media ratio of common carotid arteries after balloon injury. Data are presented as mean±S.E.M., n=5. ***, P<0.001 compared with control group. These results indicate that nstpbp5185 attenuate of neointima formation and its therapeutic potential for treating restenosis after percutaneous transluminal coronary angioplasty (PTCA).

### Aortic Root Lesion Area in Vehicle-, Aspirin-, and Nstpbp5185-Treated Apo E-Deficient Mice.

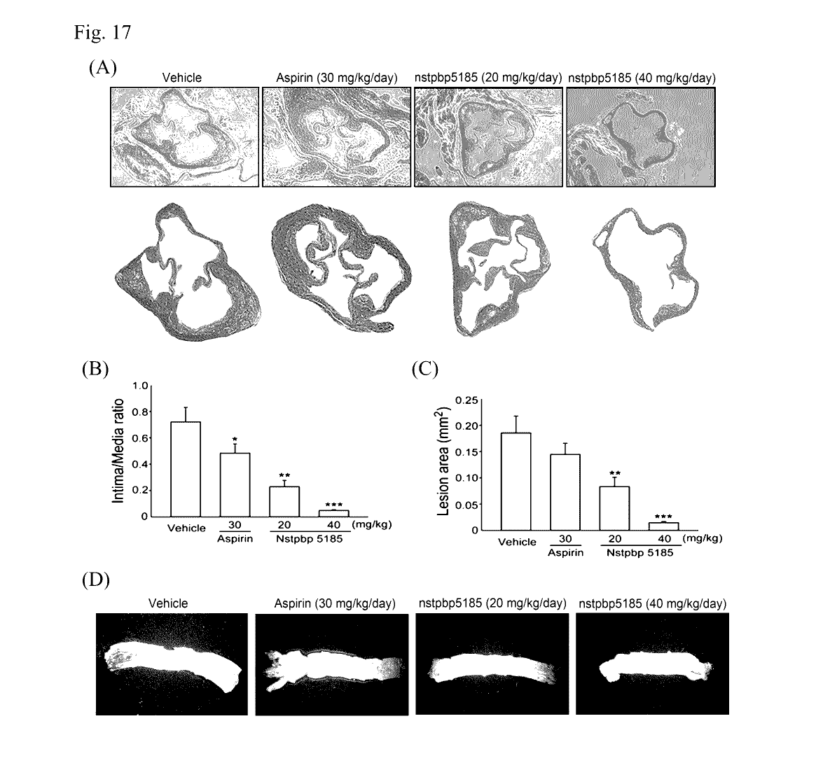

We further determined the inhibitory effect of nstpbp5185 on aortic atherosclerotic lesion area. Eight-week-old Apo E-deficient mice were orally given with vehicle, aspirin and nstpbp5185 at indicated dosage. After 12 weeks, each individual aortic root cross section was sliced, stained with Hematoxylin and Eosin (HE) and analyzed under microscopy. Representative photomicrographs of aortic root obtained from mice were shown. Original magnification was ×40. Mice were treated as described in (FIG. 17A), intima/media ratio for each individual aortic root cross section shown at the bottom (FIG. 17B) and digitized lesion area in 2 mm per cross-section in vehicle-, aspirin-, and nstpbp05185-treated Apo E-deficient mice (FIG. 17C) were then evaluated. Data are presented as the means±S.E.M. ***p<0.001, compared to the control group. (FIG. 17D) Aortic lesion areas of Apo E-deficient mice by en face preparation. Eight-week-old Apo E-deficient mice were orally given with vehicle, aspirin and nstpbp05185 at indicated dosage. After 12 weeks, aortic lesion area was then analyzed. Representative photomicrographs of aorta vessel from each group of mice were shown. Original magnification was ×10. These nstpbp5185 is an ideal safe and efficacious agent for preventing the progression of atherosclerogenic lesion by virtue of its TP antagonism through antipletelet and anti-inflammatory activities.

### Manifestation of Allergen-Induced Asthmatic Mice

Mice challenged with OVA inhalation showed obvious signs of sickness, including sneezes, nose rubbing, breathing deeply and fast, lip and eye cyanosis, ruffled fur, forelimb shrinkage lift, stooping, irritability, and other various degrees of asthma immediate responses. These symptoms persisted in mice treated with PBS (Group 2); however, in mice treated with nstpbp5185 (Group 3 and Group 4) alleviated symptoms were observed. The mice in the control group (Group 1) did not show any of the above symptoms.

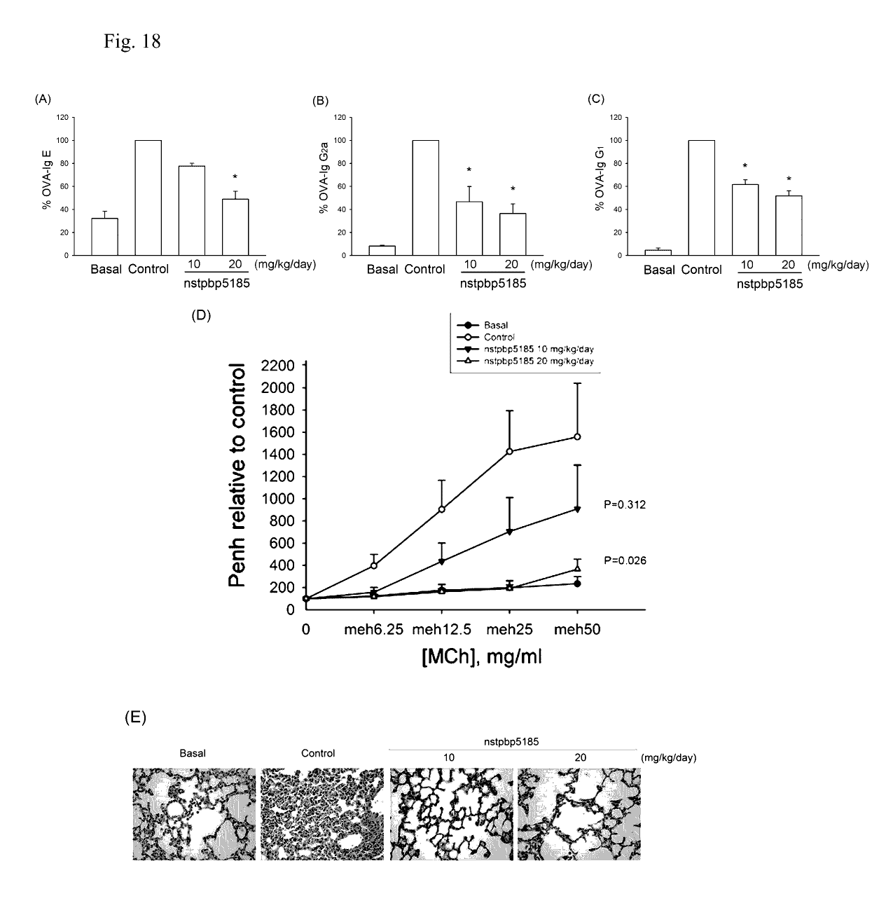

We further analyzed the production of OVA-specific IgE, IgG1, and IgG2a in serum; the former two isotypes are indicators of Th2-skewed inflammation and IgG2a is a marker of Th1-skewed inflammation. As shown in FIG. 18, OVA immunization and challenge induced a significant production of OVA-specific IgE (FIG. 18A), IgG2a (FIG. 18B), and IgG1 (FIG. 18C) antibodies. Nstpbp5185 treatment resulted in significant reduction in OVA-specific IgE (FIG. 18A), IgG2a (FIG. 18B), and IgG1 (FIG. 18C) levels. Together these data showed that nstpbp5185 treatment could effectively prevent the development of inflammation in OVA-sensitized/challenged mice.

### Comparison Among Aspirin, Ridogrel, S-18886 and Nstpbp5185

Although Ridogrel was considered not superior to aspirin in enhancing the fibrinolytic efficacy of streptokinase in a clinical trial recruiting patents with acute myocardial infarction, however Ridogrel might be more effective in preventing new ischemic events. The lack of superiority of Ridogrel over aspirin might be explained by (1) the comparatively modest receptor blockade induced by Ridogrel (IC₅₀, 10⁻⁶ M for TxA2 receptor vs 10⁻⁹ M for TxA2 synthase), and (2) the impaired endothelial capacity to produce PGI2 in atherosclerotic arteries. Currently, the concept holds that endothelial dysfunction, platelet hyperactivity, and inflammation play a role in atherogenesis. Inhibition of thromboxane A2 receptor may improve endothelial function and reduce the inflammatory component of atherosclerosis in addition to the well-demonstrated antiplatelet activity. The TP receptors are not only stimulated by TxA2 but also by virtually all eicosanoids such as isoprospanes which are capable of inducing platelet aggregation, vasoconstriction, stimulating the expression of adhesion molecules such as ICAM-1 in endothelial cells, thus leading to leucocyte adherence, accelerating progression of atherosclerotic lesions. Thus, a new selective TP receptor antagonist, S-18886, exhibits advantage over aspirin in improving flow mediated Ach-induced vasodilatation, suggesting endogenous agonists of TP receptors may contribute to endothelial dysfunction in patient with atherosclerosis. As compared to Ridogrel and S-18886, nstpbp5185 exerts its antithrombotic activity through its thromboxane A2 receptor blockade (IC₅₀, 10⁻⁷ M) and thromboxane synthase inhibition (IC50, 10⁻⁵ M)(Table 3). Thus its action profile is more close to S-18886, a selective receptor blocker in addition to possible TxA2 synthase inhibition as the administered dose is high. However, the safety profile shows that nstpbp5185 and Ctkf6f2, at 10 M, exert little effects on receptor tyrosine kinases and a high specificity toward TP receptor, rendering a favored low-toxicity trend for further development.

TABLE III
Comparison among Aspirin,
Ridogrel, S18886 and nstpbp5185

| | | Thromboxane | Thromboxane | PGI₂ |
|---|---|---|---|---|
| | Cox-1,-2 | synthase | receptor | formation |
| Aspirin | ↓ | ± | ± | ↓ |
| Ridogrel | ± | ↓(10⁻⁴M) | ↓(10⁻⁵M) | ↑ |
| S18886 | ± | ↓ | ↓ | ± |
| Compound | ± | ↓(10⁻⁶M) | ↓(10⁻⁷M) | ND |
| 5185 | | | | |

### Effect of Ctkf6f2 on Tail Bleeding Time in Mouse Model

The prolongation of the bleeding time is the common undesirable side effect of antithrombotic medications. Whether Ctkf6f2 would affect hemostasis function in vivo was evaluated by the tail transaction model. As shown in Table 4, the average bleeding time of vehicle-treated control group was 77±6.4 sec. The group treated with compound 5185 (10 μg/g) or Ctkf6f2 (10 μg/g), does not show apparent change on the average bleeding time, with value of 76.5±9.0 and 82.8±5.4 sec, respectively, whereas aspirin and tirofiban prolonged significantly the tail bleeding time.

TABLE IV
Effect of Ctkf6f2 on tail bleeding time in mice.

| | Dosage (μg/g) | Tail bleeding time (sec) |
|---|---|---|
| DMSO (control) | | 77 ± 6.4 |
| Aspirin | 150 μg/g | 433 ± 19.1*** |
| tirofiban | 0.4 μg/g | 271 ± 15.9*** |
| nstpbp5185 | 10 μg/g | 76.5 ± 9.0 |
| Ctkf6f2 | 10 μg/g | 82.8 ± 5.4 |
| | 30 μg/g | 136.0 ± 4.9* |

Values are presented as means ± SEM (n = 4).
*p < 0.05,
**p < 0.01, and
***p < 0.001 as compared with the control, one-way ANOVA (Newman-Keuls test).

The in vivo giving dosages of aspirin, compound 5185, Ctkf6f2 and tirofiban were calculated according to IC₅₀ of each compound on platelet aggregation caused by U46619 (1 μM). Effect of ctkf6f2 and others on tail bleeding time was measured at 10 min after intravenous injection of vehicle control, aspirin (150 μg/g), nstpbp5185 (10 μg/g), ctkf6f2 (10 μg/g) and high dose of Ctkf6f2 (30 μg/g). Data are presented as the mean±S.E.M. (n=4). ***p<0.001 as compared with the control, one-way ANOVA (Newman-Keuls multiple comparison test). Even when given Ctkf6f2 dose up to (30 μg/g), the average bleeding time was only increased to 136.0±4.9 sec. Since aspirin is the most popular antiplatelet drug so far with the notorious side effect—bleeding time prolongation, the outcome of aspirin was also measured. In contrast, the group of aspirin (150 μg/g) showed a significant increase on bleeding time with the value of 433±19.1 sec, which is about 5-fold longer than the control, and the tirofiban (0.4 μg/g) also showed a 3-fold longer than the control group (271±15.9 sec).

### Effects of Nstpbp5185 on U46619-Stimulated Platelet Protein Tyrosine Phosphorylation

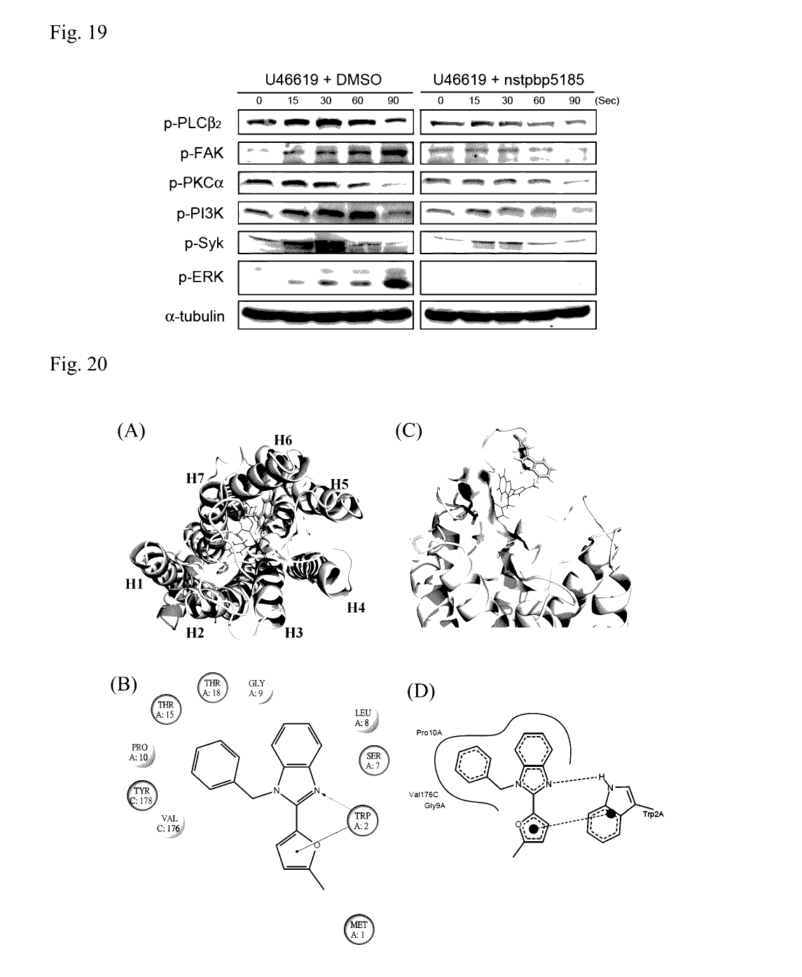

Given the thromboxane receptor-blocking effect of nstpbp5185, the effect of nstpbp5185 on U46619-stimulated signal transduction in platelets was investigated. Stimulation of thromboxane receptors results in phosphorylation of PLC_{β2} and activates downstream signaling pathway PKCα, PI3K, Syk, extracellular signal-regulated kinase (ERK) and focal adhesion kinase. The effect of nstpbp5185 on levels of protein tyrosine phosphorylation was examined in platelet cell lysates in different time course. FIG. 19 showed a time-dependent tyrosine phosphorylation of platelets stimulated with U46619 (1 μM) in the absence or presence of nstpbp5185. In the presence of nstpbp5185 (2.5 μM), the overall pattern of protein tyrosine phosphorylation of signaling molecules during platelet activation was suppressed, including phospholipase C_{β2} (PLC_{β2}), FAK, PKCα, PI3-kinase, Syk and ERK.

Docking Studies of Nstpbp5185 with Thromboxane Receptor

The docking analysis demonstrated that nstpbp5185 can bind to the modeled thromboxane receptor with similar binding affinity of U46619, and a higher affinity than that of TXA₂ and aspirin. Trp2 in the docking model of nstpbp5185 with TXA receptor forms hydrogen bonds and pi-pi stacking interaction with nstpbp5185. Gly9, Pro10, and Val176 play the key roles of hydrophobic contact. The docking pose is overall maintained by hydrogen bonds, pi-pi stacking interaction, and hydrophobic contacts (FIG. 20).

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A substituted benzimidazole having a chemical structure (I): 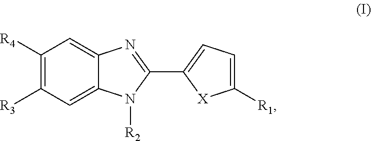
wherein X is selected from oxygen, carbon, or sulfur; R₁ is C₁₋₆ alkyl;
R₂ is selected from hydrogen, C₁₋₆ alkyl, C₁₋₆ alkyl pyridine, unsubstituted benzyl, or benzyl substituted with one or more C₁₋₆ alkyl, C₁₋₆ alkoxy, halo, or nitro; and
R₃ and R₄ are independently selected from hydrogen, C₁₋₆ alkoxy, halogen, phenylcarbonyl, or C₁₋₆ alkylcarbonyl.

2. The substituted benzimidazole of claim 1, wherein X is oxygen; and R₁ is methyl.

3. The substituted benzimidazole of claim 1, wherein the benzyl substituted with one or more C₁₋₆ alkyl, C₁₋₆ alkoxy, halo, or nitro is in ortho-position, meta-position, or para-position.

4. A pharmaceutical composition comprising:
an effective amount of a compound having a chemical structure (I): 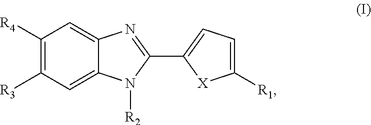
wherein X is selected from oxygen, carbon, or sulfur; R₁ is C₁₋₆ alkyl;
R₂ is selected from hydrogen, C₁₋₆ alkyl, C₁₋₆ alkyl pyridine, unsubstituted benzyl, or benzyl substituted with one or more C₁₋₆ alkyl, C₁₋₆ alkoxy, halo, or nitro; and
R₃ and R₄ are independently selected from hydrogen, C₁₋₆ alkoxy, halogen, phenylcarbonyl, or C₁₋₆ alkylcarbonyl.

5. The pharmaceutical composition of claim 4, wherein X is oxygen; and R₁ is methyl.

6. The pharmaceutical composition of claim 4, wherein the benzyl substituted with one or more C₁₋₆ alkyl, C₁₋₆ alkoxy, halo, or nitro is in ortho-position, meta-position, or para-position.

7. The pharmaceutical composition of claim 4, wherein the compound having a chemical structure (I) is:

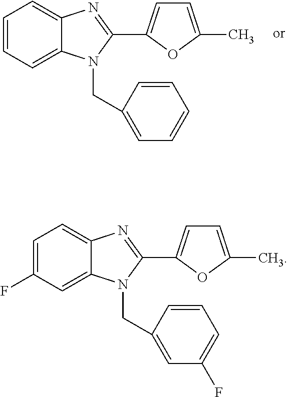 [SMILES: Cc1ccc(-c2nc3ccccc3n2Cc2ccccc2)o1 ; "or" ; Cc1ccc(-c2nc3ccc(F)cc3n2Cc2cccc(F)c2)o1]

8. A method of preventing or treating a subject suffering from diseases associated with thromboxane A₂ by inhibiting thromboxane A₂ activity, comprising: administering an effective amount of a compound having a chemical structure (I) to the subject: 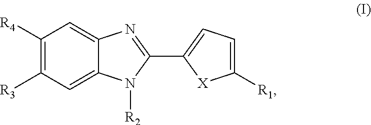
wherein X is selected from oxygen, carbon, or sulfur; R₁ is C₁₋₆ alkyl;
R₂ is selected from hydrogen, C₁₋₆ alkyl, C₁₋₆ alkyl pyridine, unsubstituted benzyl, or benzyl substituted with one or more C₁₋₆ alkyl, C₁₋₆ alkoxy, halo, or nitro; and
R₃ and R₄ are independently selected from hydrogen, C₁₋₆ alkoxy, halogen, phenylcarbonyl, or C₁₋₆ alkylcarbonyl.

9. The method of claim 8, wherein the diseases associated with thromboxane A₂ comprises inflammatory diseases, coronary artery diseases, percutaneous transluminal coronary angioplasty, and diseases associated with platelet activation aggregation and/or platelet activation.

10. The method of claim 8, wherein X is oxygen; and R₁ is methyl.

11. The method of claim 8, wherein the benzyl substituted with one or more C₁₋₆ alkyl, C₁₋₆ alkoxy, halo, or nitro is in ortho-position, meta-position, or para-position.

12. The method of claim 8, wherein the compound having a chemical structure (I) is:

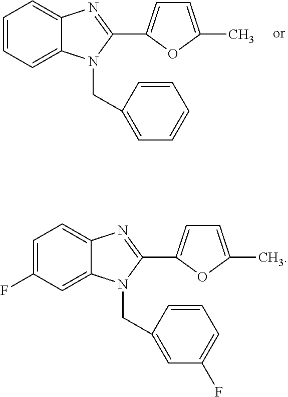 [SMILES: Cc1ccc(-c2nc3ccccc3n2Cc2ccccc2)o1 ; "or" ; Cc1ccc(-c2nc3ccc(F)cc3n2Cc2cccc(F)c2)o1]

13. The method of claim 8, wherein the diseases associated with platelet activation aggregation and/or platelet activation comprise thrombosis, established peripheral arterial disease, thrombophlebitis, arterial embolism, coronary and cerebral arterial thrombosis, unstable angina, myocardial infarction, stroke, cerebral embolism, renal embolism, pulmonary embolism, unstable angina, myocardial infarction, thrombotic stroke, or peripheral vascular disease.

14. The method of claim 8, wherein the inflammatory diseases comprise asthma, and atheroscelrosis.
